(19) United States
(12) Patent Application Publication
(10) Pub. No.: US 2024/0191214 A1
(43) Pub. Date: Jun. 13, 2024

(54) RECOMBINANT PRENYLTRANSFERASE POLYPEPTIDES ENGINEERED FOR ENHANCED BIOSYNTHESIS OF CANNABINOIDS

(71) Applicants: WILLOW BIOSCIENCES, INC., Calgary (CA); EPIMERON USA, INC., Sunnyvale, CA (US)

(72) Inventors: Trish CHOUDHARY, Belmont, CA (US); Xueyang FENG, Fremont, CA (US); Gisele PASSAIA PRIETSCH, Vancouver (CA)

(21) Appl. No.: 18/424,333

(22) Filed: Jan. 26, 2024

Related U.S. Application Data
(63) Continuation of application No. PCT/US2022/074264, filed on Jul. 28, 2022.
(60) Provisional application No. 63/227,747, filed on Jul. 30, 2021.

(51) Int. Cl. C12N 9/10; C12N 15/52; C12N 15/81; C12P 7/42; C12P 17/06

(52) U.S. Cl. CPC C12N 9/1085; C12N 15/52; C12N 15/81; C12P 7/42; C12P 17/06; C12Y 205/0101

(57) ABSTRACT

The present disclosure relates to recombinant polypeptides that have prenyltransferase activity, nucleic acids encoding these recombinant polypeptides, recombinant host cells that produce these recombinant polypeptides, and compositions comprising the recombinant polypeptides, nucleic acids, and/or recombinant host cells. The present disclosure also relates to uses of these recombinant polypeptides, nucleic acids encoding them, and recombinant host cells comprising them, in methods for the preparation of cannabinoids.

### CROSS REFERENCE TO RELATED APPLICATIONS

This application is a continuation of International Appl. No. PCT/US2022/074264, filed Jul. 28, 2022, which claims priority of U.S. Provisional Patent Appl. No. 63/227,747, filed Jul. 30, 2021, the entirety of which is hereby incorporated by reference herein.

### FIELD

The present disclosure relates to recombinant prenyltransferase polypeptides engineered with enhanced activity and the use of recombinant genes encoding these polypeptides in recombinant host cell systems for the production of cannabinoid compounds.

### REFERENCE TO SEQUENCE LISTING

The official copy of the Sequence Listing is submitted concurrently with the specification via USPTO Patent Center as an WIPO Standard ST.26 formatted XML file with file name “13421-014WO1.xml”, a creation date of Jul. 27, 2022, and a size of 1,010,622 bytes. This Sequence Listing filed via USPTO Patent Center is part of the specification and is incorporated in its entirety by reference herein.

### BACKGROUND

Cannabinoids are a class of compounds that act on endocannabinoid receptors and include the phytocannabinoids naturally produced by Cannabis sativa. Cannabinoids include the more prevalent and well-known compounds, Δ⁹-tetrahydrocannabinol (THC), cannabidiol (CBD), as well as 80 or more less prevalent cannabinoids, cannabinoid precursors, related metabolites, and synthetically produced derivative compounds. Cannabinoids are increasingly used to treat a range of diseases and conditions such as multiple sclerosis and chronic pain. Current large-scale production of cannabinoids for pharmaceutical or other use is through extraction from plants. These plant-based production processes, however, have several challenges including susceptibility of the plants to inconsistent production caused by variance in biotic and abiotic factors, difficulty reproducing identical cannabinoid accumulation profiles, and difficulty in producing a single cannabinoid compound with purity high enough for pharmaceutical applications. While some cannabinoids can be produced as a single pure product via chemical synthesis, these processes have proven very costly and too costly for large-scale production.

More economical biosynthetic approaches to cannabinoid production are being developed using microbial hosts. These processes have the potential to be robust, scalable, and capable of producing single cannabinoid compound with higher purity compared to other current processes. Several biosynthetic systems for cannabinoid compound have been reported (see e.g., WO2019071000, WO2018200888, WO2018148849, WO2019014490, US20180073043, US20180334692, and WO2019046941). These biosynthetic systems are capable of producing the cannabinoid, CBGA, to some extent, but are not capable of efficient production of the downstream cannabinoid compounds, CBDA and THCA.

There exists a need for improved recombinant polypeptides with enhanced prenyltransferase activity, recombinant host cells with genes expressing these polypeptides, and methods for their use in the biosynthetic production of cannabinoid compounds, such as CBGA, CBGVA, CBDA, THCA, and CBCA.

### SUMMARY

The present disclosure relates generally to recombinant polypeptides engineered with increased prenyltransferase activity relative to the naturally occurring prenyltransferase from Cannabis sativa, and the use of these recombinant polypeptides in recombinant host cell systems and methods for the preparation of cannabinoids. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides a recombinant polypeptide having prenyltransferase activity, wherein the polypeptide comprises an amino acid sequence of at least 80% identity to SEQ ID NO: 20, and an amino acid residue difference as compared to SEQ ID NO: 20 at one or more positions selected from: W61, F64, I79, F134, W153, F158, S175, S177, T180, N235, E284, and A293; optionally, wherein the amino acid differences are selected from: W61A, W61V, F64G, F64L, F64M, F64T, F64W, I179A, I79C, I79N, I79S, F134G, F134V, W153L, F158A, F158G, F158S, S175A, S175G, S175T, S175V, Y176S, S177A, S177G, S177T, T180I, T180L, T180R, T180V, N235C, N235K, N235V, E284D, E284K, E284R, A293G, A293K, and A293V.

In at least one embodiment, the polypeptide further comprises an amino acid sequence of at least 80% identity to SEQ ID NO: 20, and an amino acid residue difference as compared to SEQ ID NO: 20 at one or more positions selected from: P5, H7, D10, N11, K34, C41, R46, F49, N50, R52, L54, G58, F65, V68, F75, M80, D87, I91, K93, D95, V99, I105, E106, I113, V115, I121, T123, K125, A129, F138, I140, F144, F161, I165, F173, Y176, S181, V188, R190, F193, S194, F195, I196, I197, M200, G204, M205, S214, E217, D219, T229, F238, S241, V243, L249, S251, S253, W258, S264, M267, F276, C277, L278, F280, Q281, T282, A286, L287, A288, Y290, A291, P294, S295, F299, F301, I302, W303, L304, L305, Y307, A308, E309, Y310, F311, V312, Y313, V314, and F315; optionally, wherein the amino acid differences are selected from: P5G, P5V, H7C, D10L, D10V, D10W, N11D, K34E, C41A, C41G, C41S, R46K, F49L, F49M, F49R, N50D, R52P, L54S, G58S, F65L, V68D, F75W, M80V, D87E, I91V, K93N, D95N, V99A, I105V, E106R, I113N, I113W, V115A, I121T, T123K, K125M, K125V, K125W, A129T, F138I, I140T, F144S, F161V, I165L, I165T, F173I, Y176S, S181R, V188A, V188S, R190A, R190G, R190Q, R190S, F193L, S194A, S194L, S194V, F195V, I196T, I197T, M200R, G204A, G204S, M205G, M205R, S214C, E217G, D219V, T229V, F238L, F238W, S241F, V243A, L249A, L249V, S251A, S251C, S253P, W258R, S264Y, M267T, F276L, C277A, C277M, C277R, L278P, F280G, F280L, F280R, Q281R, T282P, A286G, L287F, A288P, Y290S, A291E, P294E, S295A, F299L, F301S, I302L, W303C, L304R, L305S, Y307H, Y307S, A308E, A308P, A308R, E309V, Y310C, Y310P, Y310S, F311P, F311S, V312G, Y313H, Y313P, V314A, and F315S.

In at least one embodiment, the polypeptide comprises a combination of amino acid differences as compared to SEQ ID NO: 20 as found in any one of the polypeptides of even-numbered SEQ ID NO: 22-514 and/or as described in Table 3 herein.

In at least one embodiment, the polypeptide comprises an amino acid sequence of at least 80%, at least 85%, at least 90%, at least 95%, at least 97%, at least 98%, or at least 99% identity to a sequence selected from the group consisting of SEQ ID NO: 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, and 514.

In at least one embodiment, the prenyltransferase activity of the polypeptide as compared to a polypeptide consisting of SEQ ID NO: 20 is encoded by a polynucleotide sequence having at least 80% identity to SEQ ID NO: 19, and a silent codon difference as compared to SEQ ID NO: 19 at a position encoding an amino acid residue selected from: V33, I37, F73, N74, A78, Q82, K93, P97, V99, S104, L111, L117, G119, F132, V133, I137, G139, F141, R152, Q155, N160, S166, A182, T201, G218, I213, V224, S225, A233, G242, V261, K263, F276, S295, L304, Y306, F311, and V312; optionally, wherein the codon differences are selected from: V33 (GTT>GTC), I37 (ATT>ATC), F73 (TTT>TTC), N74 (AAT>AAC), A78 (GCA>GCG), Q82 (CAA>CAG), K93 (AAG>AAA), P97 (CCA>CCG), V99 (GTT>GTC), S104 (TCA>TCT), L111 (TTA>TTG), L117 (TTG>CTG), G119 (GGT>GGC), F132F (TTC>TTT), V133 (GTT>GTC), G139 (GGT>GGG), R152 (AGA>CGT), Q155 (CAA>CAG), N160 (AAT>AAC), L162 (TTG>CTG), S166 (TCT>TCC), A182 (GCA>GCC), T201 (ACT>ACG), I213 (ATC>ATT), G218 (GGT>GGG), V224 (GTT>GTC), S225 (TCA>TCG), A233 (GCA>GCG), G242 (GGT>GGC), V261 (GTT>GTC), K263 (AAA>AAG), F276 (TTC>TTT), S295 (TCA>TCT), L304 (TTG>CTG), Y306 (TAT>TAC), F311 (TTT>TTC), and V312 (GTT>GTC).

In at least one embodiment, the polypeptide comprises an N-terminal truncation of from 2 to 12 amino acids as compared to SEQ ID NO: 20; optionally, wherein, the polypeptide comprises an amino acid sequence of at least 80%, at least 85%, at least 90%, at least 95%, at least 97%, at least 98%, or at least 99% identity to a sequence selected from the group consisting of SEQ ID NO: 516, 518, 520, 522, and 524.

In at least one embodiment, the prenyltransferase activity of the polypeptide as compared to a polypeptide consisting of SEQ ID NO: 20 is increased at least 1.2-fold, at least 1.5-fold, at least 2-fold, at least 5-fold, or more. In at least one embodiment, the prenyltransferase activity of the polypeptide is measured as the rate of conversion of the substrates olivetolic acid (OA) and geranyl pyrophosphate (GPP) to cannabigerolic acid (CBGA).

In at least one embodiment, the prenyltransferase activity of the polypeptide when expressed in a recombinant host cell comprising a pathway capable of producing olivetolic acid (OA) results in a titer of cannabigerolic acid (CBGA) produced by the cell that is increased relative to a control cell by at least 1.2-fold, at least 1.5-fold, at least 2-fold, at least 5-fold, or more.

In at least one embodiment, the present disclosure also provides a polynucleotide encoding a recombinant polypeptide having prenyltransferase activity of the present disclosure. In at least one embodiment, the polynucleotide comprises: (a) a sequence of at least 80%, at least 85%, at least 90%, at least 95%, at least 97%, at least 98%, or at least 99% identity to a sequence selected from the group consisting of SEQ ID NO: 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161,163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, and 513; (b) a codon degenerate sequence of a sequence selected from the group consisting of SEQ ID NO: 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121,123, 125, 127, 129, 131,133, 135, 137, 139, 141,143, 145, 147, 149, 151, 153, 155, 157, 159, 161, 163, 165, 167, 169, 171, 173, 175, 177, 179, 181, 183, 185, 187, 189, 191, 193, 195, 197, 199, 201,203, 205, 207, 209, 211,213, 215, 217, 219, 221,223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, and 513.

In at least one embodiment, the present disclosure also provides an expression vector comprising a polynucleotide encoding a recombinant polypeptide having prenyltransferase activity of the present disclosure, optionally wherein, the expression vector comprises a control sequence.

In at least one embodiment, the present disclosure also provides a recombinant host cell comprising: (a) a polynucleotide encoding a recombinant polypeptide having prenyltransferase activity of the present disclosure, or (b) an expression vector comprising a polynucleotide encoding a recombinant polypeptide having prenyltransferase activity of the present disclosure.

In at least one embodiment, the present disclosure provides a method for preparing a recombinant polypeptide having prenyltransferase activity of the present disclosure wherein the method comprises culturing a recombinant host cell of the present disclosure and isolating the polypeptide from the cell.

In at least one embodiment, the present disclosure provides a method for preparing a recombinant polypeptide having prenyltransferase activity comprising:
(a) transforming a host cell with an expression vector comprising a polynucleotide encoding a recombinant polypeptide having prenyltransferase activity of the present disclosure;
(b) culturing said transformed host cell under conditions whereby said recombinant polypeptide is produced by said host cell; and
(c) recovering said recombinant polypeptide from said host cells.

In at least one embodiment, the present disclosure also provides a recombinant host cell comprising a nucleic acid encoding a recombinant polypeptide having prenyltransferase activity of the present disclosure. In at least one embodiment, the nucleic acid encodes a N-terminal fusion of the Erg20ww polypeptide of SEQ ID NO: 526 and the recombinant polypeptide having prenyltransferase activity of the present disclosure.

In at least one embodiment of the recombinant host cell, the host cell further comprises a pathway of enzymes capable of producing a cannabinoid precursor; optionally, wherein the cannabinoid precursor is divarinic acid (DA) or olivetolic acid (OA).

In at least one embodiment of the recombinant host cell, the host cell further comprises a pathway of enzymes capable of converting hexanoic acid (HA) to olivetolic acid (OA); optionally, wherein the pathway comprises enzymes capable of catalyzing reactions (i)-(iii):

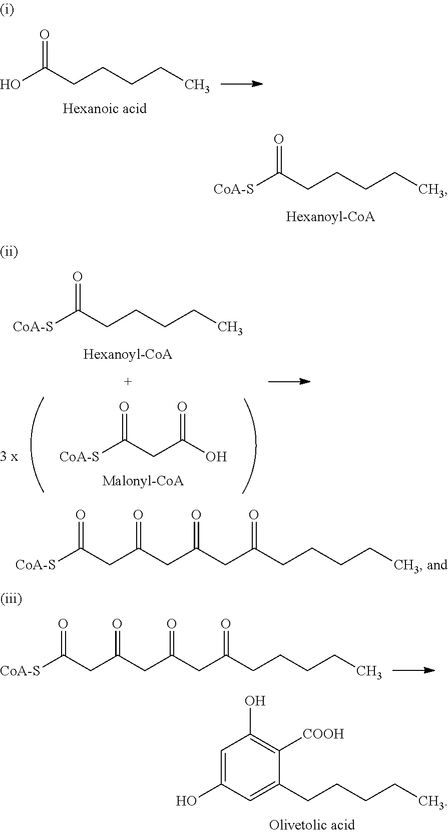

In at least one embodiment of the recombinant host cell, the host cell further comprises a pathway of enzymes capable of converting hexanoic acid (HA) to olivetolic acid (OA), wherein the pathway comprises at least the enzymes AAE, OLS, and OAC; optionally, wherein the enzymes AAE, OLS, and OAC, have an amino acid sequence of at least 90% identity to SEQ ID NO: 2 (AAE), SEQ ID NO: 4 (OLS), and SEQ ID NO: 6 (OAC), respectively.

In at least one embodiment of the recombinant host cell, the host cell further comprises a nucleic acid encoding an enzyme capable of catalyzing the conversion of CBGA to Δ⁹-THCA, CBDA, and/or CBCA; optionally, wherein the host cell further comprises a nucleic acid encoding an enzyme capable of catalyzing a reaction (v), (vi), and/or (vii):

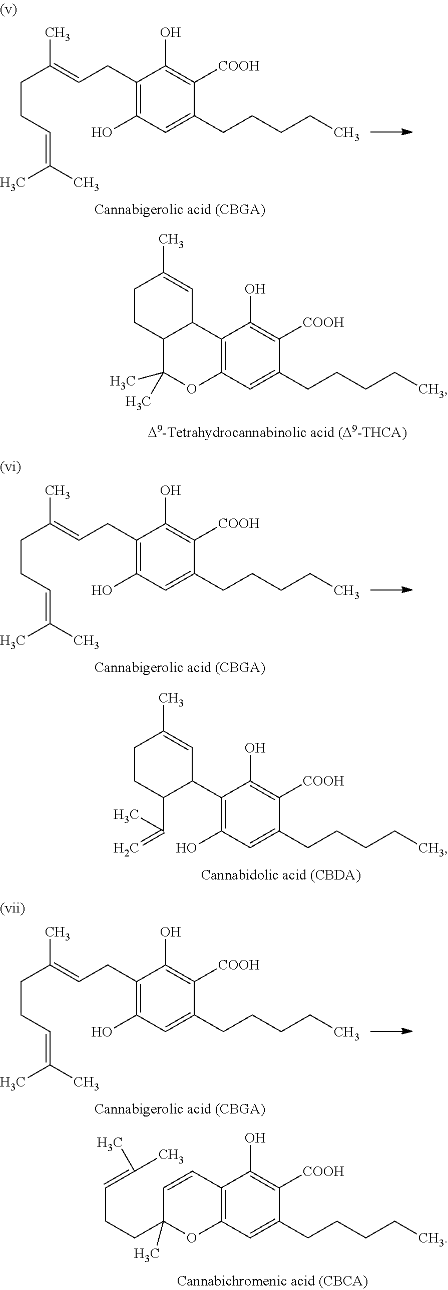

In at least one embodiment of the recombinant host cell, the host cell further comprises a nucleic acid encoding THCA synthase, CBDA synthase, and/or CBCA synthase; optionally, wherein the CBDA synthase has an amino acid sequence of at least 90% identity to SEQ ID NO: 12 or 14; and the THCA synthase having an amino acid sequence of at least 90% identity to SEQ ID NO: 16 or 18.

In at least one embodiment of the recombinant host cell, the host cell is capable of producing a cannabinoid selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁸-tetrahydrocannabinolic acid (Da-THCA), Δ⁸-tetrahydrocannabinol (Da-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.

In at least one embodiment of the recombinant host cell, the host cell comprises a pathway capable of producing CBGA, and the production of CBGA is increased at least 2-fold, at least 3-fold, at least 4-fold, at least 5-fold, or more, relative to a control recombinant host cell comprising a pathway with the recombinant polypeptide having prenyltransferase activity replaced by a polypeptide of SEQ ID NO: 20.

In at least one embodiment of the recombinant host cell, the source of the host cell is selected from Saccharomyces cerevisiae, Yarrowia lipolytica, Pichia pastoris, and Escherichia coli. In at least one embodiment, the nucleic acid is integrated in the host cell genome at a locus selected from: NDE1, XII-5, Gal80, ROQ1; optionally, wherein the nucleic acid is integrated in the host cell genome at two loci selected from: XII-5 and NDE1; or ROQ1 and NDE1.

In at least one embodiment, the present disclosure also provides a method for producing a cannabinoid comprising: (a) culturing in a suitable medium a recombinant host cell of the present disclosure; and (b) recovering the produced cannabinoid. In at least one embodiment, the method further comprises contacting a cell-free extract of the culture with a biocatalytic reagent or chemical reagent.

In at least one embodiment, the present disclosure also provides a method for preparing a compound of structural formula (I)

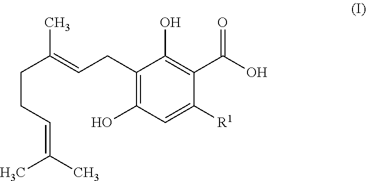

wherein, R¹ is C1-C7 alkyl; the method comprising contacting under suitable reactions conditions geranyl pyrophosphate (GPP) and a compound of structural formula (II)

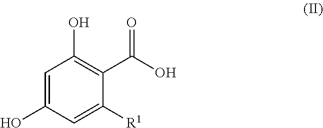

wherein, R¹ is C1-C7 alkyl, and a recombinant polypeptide having prenyltransferase activity of the present disclosure.

In at least one embodiment of the method: (a) the compound of structure formula (I) is cannabigerolic acid (CBGA) and the compound of structural formula (II) is olivetolic acid (OA); or (b) the compound of structure formula (I) is cannabigerovarinic acid (CBGVA) and the compound of structural formula (II) is divarinic acid (DA).

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts an exemplary four enzyme pathway capable of converting hexanoic acid (HA) to the cannabinoid precursor, olivetolic acid (OA), and then further converting OA to the cannabinoid, cannabigerolic acid (CBGA). The four enzymes catalyzing the steps in the biosynthetic pathway are AAE, OLS, OAC, and PT.

FIG. 2 depicts three exemplary two step pathways for converting the cannabinoid, CBGA, to one or more of the cannabinoids, Δ⁹-THCA, CBDA, and/or CBCA, and then, optionally, further converting them to the decarboxylated cannabinoids, Δ⁹-THC, CBD, and/or CBC. The first conversion from CBGA to Δ⁹-THCA, CBDA, and/or CBCA can be catalyzed by a cannabinoid synthase, CBDA synthase (CBDAS), THCA synthase (THCAS) and/or CBCA synthase (CBCAS), respectively. As described elsewhere herein, in some embodiments the single cannabinoid synthase (e.g., CBDAS) is capable of catalyzing not only the conversion of CBGA to its preferred product (e.g., CBDAS preferentially converts CBGA to CBDA), but also converts CBGA to one or both of the other cannabinoid acid products, typically in lesser amounts.

FIG. 3 depicts an exemplary four enzyme pathway capable of converting butyric acid (BA) to the rare cannabinoid precursor, divarinic acid (DA), and then further converting DA to the rare cannabinoid, cannabigerovarinic acid (CBGVA). The four enzymes catalyzing the steps in the biosynthetic pathway are AAE, OLS, OAC, and PT.

FIG. 4 depicts three exemplary two step pathways for converting the rare cannabinoid, CBGVA, to one or more of the rare cannabinoids, Δ⁹-THCVA, CBDVA, and/or CBCVA, and then, optionally, further converting them to the decarboxylated cannabinoids, Δ⁹-THCV, CBDV, and/or CBCV. The first conversion from CBGVA to Δ⁹-THCVA, CBDVA, and/or CBCVA can be catalyzed by a single cannabinoid synthase, CBDAs, THCAs and/or CBCAs, respectively. As described elsewhere herein, in some embodiments the single cannabinoid synthase (e.g., CBDAs) is capable of catalyzing not only the conversion of CBGVA to its preferred product (e.g., CBDAs preferentially converts CBGVA to CBDVA), but also converts CBGVA to one or both of the other cannabinoid acid products, typically in lesser amounts.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### Definitions

“Cannabinoid” refers to a compound that acts on cannabinoid receptor, and is intended to include the endocannabinoid compounds that are produced naturally in animals, the phytocannabinoid compounds produced naturally in cannabis plants, and the synthetic cannabinoids compounds. Cannabinoids as referenced in the present disclosure include, but are not limited to, the exemplary naturally occurring and synthetic cannabinoid product compounds shown below in Table 1 (below).

TABLE 1
Exemplary cannabinoid product compounds

| | Abbrev. | |
|---|---|---|
| Compound Name | Name | Chemical Structure |
| cannabigerolic acid | CBGA | 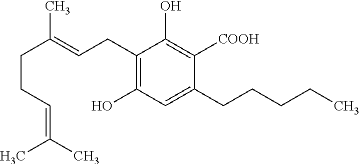 [SMILES: CCCCCc1cc(O)c(C/C=C(\C)CCC=C(C)C)c(O)c1C(=O)O] |
| cannabigerol | CBG | 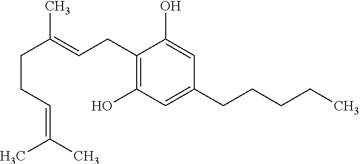 [SMILES: CCCCCc1cc(O)c(C/C=C(\C)CCC=C(C)C)c(O)c1] |
| Δ⁹-tetrahydrocannabinolic acid | Δ⁹-THCA | 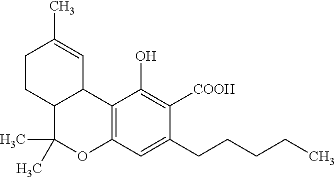 [SMILES: CCCCCc1cc2c(c(O)c1C(=O)O)C1C=C(C)CCC1C(C)(C)O2] |
| Δ⁹-tetrahydrocannabinol | Δ⁹-THC | 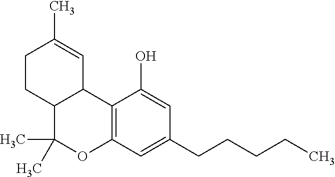 [SMILES: CCCCCc1cc(O)c2c(c1)OC(C)(C)C1CCC(C)=CC21] |
| Δ⁸-tetrahydrocannabinolic acid | Δ⁸-THCA | 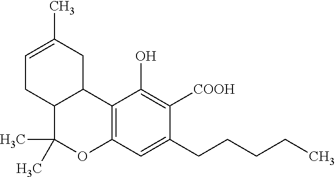 [SMILES: CCCCCc1cc2c(c(O)c1C(=O)O)C1CC(C)=CCC1C(C)(C)O2] |
| Δ⁸-tetrahydrocannabinol | Δ⁸-THC | 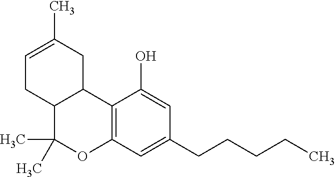 [SMILES: CCCCCc1cc(O)c2c(c1)OC(C)(C)C1CC=C(C)CC21] |
| cannabidiolic acid | CBDA | 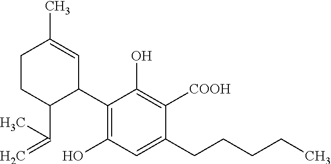 [SMILES: C=C(C)C1CCC(C)=CC1c1c(O)cc(CCCCC)c(C(=O)O)c1O] |
| cannabidiol | CBD | 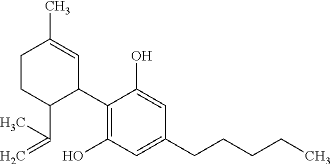 [SMILES: C=C(C)C1CCC(C)=CC1c1c(O)cc(CCCCC)cc1O] |
| cannabichromenic acid | CBCA | 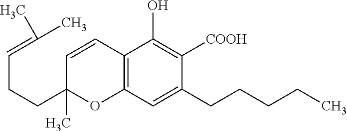 [SMILES: CCCCCc1cc2c(c(O)c1C(=O)O)C=CC(C)(CCC=C(C)C)O2] |
| cannabichromene | CBC | 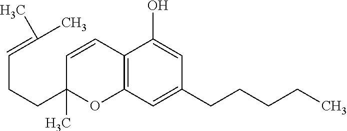 [SMILES: CCCCCc1cc(O)c2c(c1)OC(C)(CCC=C(C)C)C=C2] |
| cannabinolic acid | CBNA | 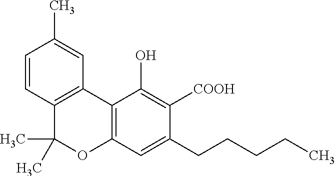 [SMILES: CCCCCc1cc2c(c(O)c1C(=O)O)-c1cc(C)ccc1C(C)(C)O2] |
| cannabinol | CBN | 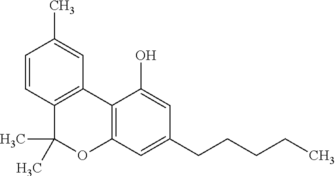 [SMILES: CCCCCc1cc(O)c2c(c1)OC(C)(C)c1ccc(C)cc1-2] |
| cannabidivarinic acid | CBDVA | 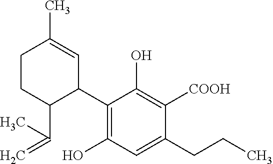 [SMILES: C=C(C)C1CCC(C)=CC1c1c(O)cc(CCC)c(C(=O)O)c1O] |
| cannabidivarin | CBDV | 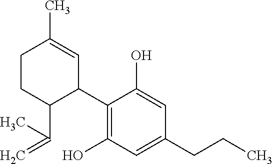 [SMILES: C=C(C)C1CCC(C)=CC1c1c(O)cc(CCC)cc1O] |
| Δ⁹-tetrahydrocannabivarinic acid | Δ⁹- THCVA | 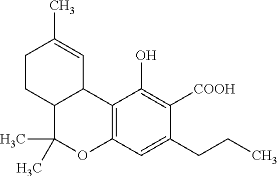 [SMILES: CCCc1cc2c(c(O)c1C(=O)O)C1C=C(C)CCC1C(C)(C)O2] |
| Δ⁹-tetrahydrocannabivarin | Δ⁹-THCV | 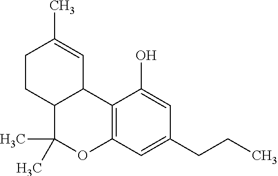 [SMILES: CCCc1cc(O)c2c(c1)OC(C)(C)C1CCC(C)=CC21] |
| cannabidibutolic acid | CBDBA | 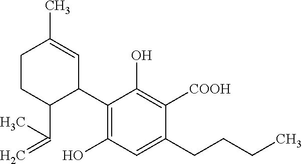 [SMILES: C=C(C)C1CCC(C)=CC1c1c(O)cc(CCCC)c(C(=O)O)c1O] |
| cannabidibutol | CBDB | 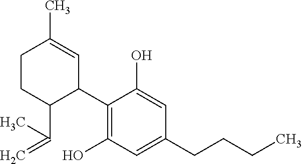 [SMILES: C=C(C)C1CCC(C)=CC1c1c(O)cc(CCCC)cc1O] |
| Δ⁹-tetrahydrocannabutolic acid | Δ⁹- THCBA | 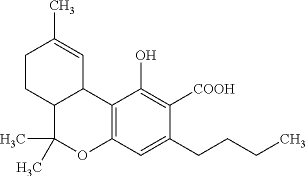 [SMILES: CCCCc1cc2c(c(O)c1C(=O)O)C1C=C(C)CCC1C(C)(C)O2] |
| Δ⁹-tetrahydrocannabutol | Δ⁹-THCB | 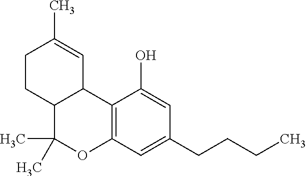 [SMILES: CCCCc1cc(O)c2c(c1)OC(C)(C)C1CCC(C)=CC21] |
| cannabigerophorolic acid | CBGPA | 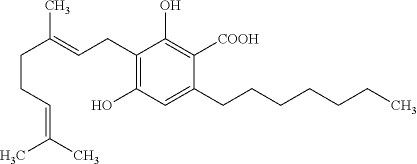 [SMILES: CCCCCCCc1cc(O)c(C/C=C(\C)CCC=C(C)C)c(O)c1C(=O)O] |
| cannabigerophorol | CBGP | 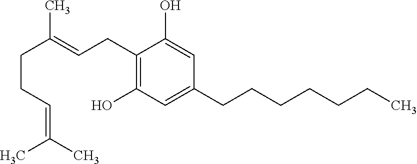 [SMILES: CCCCCCCc1cc(O)c(C/C=C(\C)CCC=C(C)C)c(O)c1] |
| cannabidiphorolic acid | CBDPA | 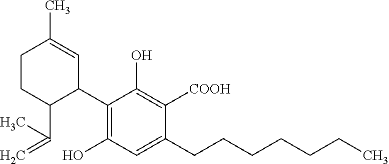 [SMILES: C=C(C)C1CCC(C)=CC1c1c(O)cc(CCCCCCC)c(C(=O)O)c1O] |
| cannabidiphorol | CBDP | 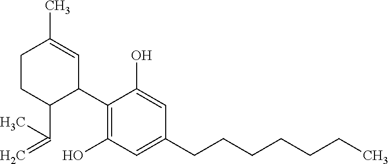 [SMILES: C=C(C)C1CCC(C)=CC1c1c(O)cc(CCCCCCC)cc1O] |
| Δ⁹- tetrahydrocannabidphorolic acid | Δ⁹- THCPA | 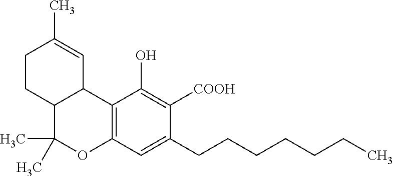 [SMILES: CCCCCCCc1cc2c(c(O)c1C(=O)O)C1C=C(C)CCC1C(C)(C)O2] |
| Δ⁹-tetrahydrocannabiphorol | Δ⁹-THCP | 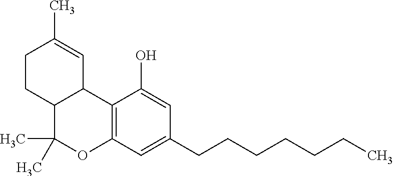 [SMILES: CCCCCCCc1cc(O)c2c(c1)OC(C)(C)C1CCC(C)=CC21] |
| cannabichromevarinic acid | CBCVA | 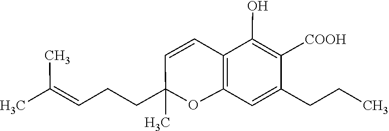 [SMILES: CCCc1cc2c(c(O)c1C(=O)O)C=CC(C)(CCC=C(C)C)O2] |
| cannabichromevarin | CBCV | 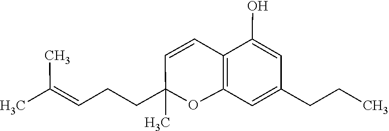 [SMILES: CCCc1cc(O)c2c(c1)OC(C)(CCC=C(C)C)C=C2] |
| cannabigerovarinic acid | CBGVA | 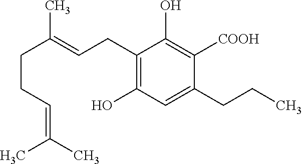 [SMILES: CCCc1cc(O)c(C/C=C(\C)CCC=C(C)C)c(O)c1C(=O)O] |
| cannabigerovarin | CBGV | 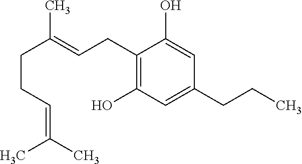 [SMILES: CCCc1cc(O)c(C/C=C(\C)CCC=C(C)C)c(O)c1] |
| cannabicyclolic acid | CBLA | 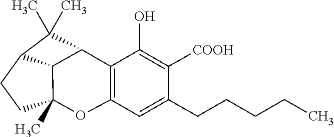 |
| cannabicyclol | CBL | 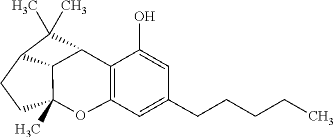 |
| cannabielsoinic acid | CBEA | 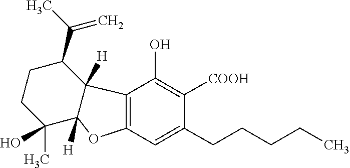 [SMILES: C=C(C)[C@@H]1CC[C@](C)(O)[C@H]2Oc3cc(CCCCC)c(C(=O)O)c(O)c3[C@@H]12] |
| cannabielsoin | CBE | 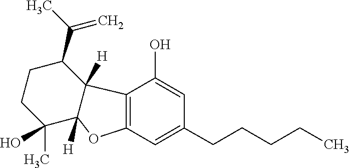 [SMILES: C=C(C)[C@@H]1CC[C@](C)(O)[C@H]2Oc3cc(CCCCC)cc(O)c3[C@@H]12] |
| cannabicitranic acid | CBTA | 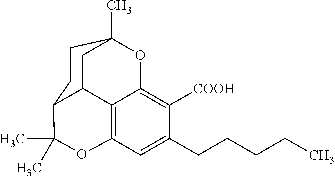 [SMILES: CCCCCc1cc2c3c(c1C(=O)O)OC1(C)CCC(C3C1)C(C)(C)O2] |
| cannabicitran | CBT | 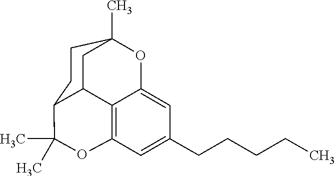 [SMILES: CCCCCc1cc2c3c(c1)OC(C)(C)C1CCC(C)(CC31)O2] |

“Pathway” refers an ordered sequence of enzymes that act in a linked series to convert an initial substrate molecule into final product molecule. As used herein, “pathway” is intended to encompass naturally-occurring pathways and non-naturally occurring, recombinant pathways. Accordingly, a pathway of the present disclosure can include a series of enzymes that are naturally-occurring and/or non-naturally occurring, and can include a series of enzymes that act in vivo or in vitro.

“Pathway capable of producing a cannabinoid” refers to a pathway that can convert a cannabinoid precursor molecule, such as hexanoic acid, into a cannabinoid product molecule, such as cannabigerolic acid (CBGA). For example, the four enzymes AAE, OLS, OAC, and PT which convert hexanoic acid to CBGA, form a pathway capable of producing a cannabinoid.

“Cannabinoid precursor” as used herein refers to a compound capable of being converted into a cannabinoid by a pathway capable producing a cannabinoid. Cannabinoid precursors as referenced in the present disclosure include, but are not limited to, the exemplary naturally occurring and synthetic cannabinoid precursors with varying alkyl carbon chain lengths summarized in Table 2 (below).

TABLE 2
Exemplary cannabinoid precursor compounds

| | Abbrev. | |
|---|---|---|
| Compound Name | Name | Chemical Structure |
| Orcinolic acid (2,4-dihydroxy-6- methylbenzoic acid) | OrcA | 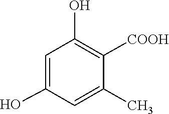 |
| Divarinic acid (2,4-dihydroxy-6- propylbenzoic acid) | DA | 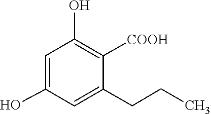 |
| Butolic acid (2-butyl-4,6- dihydroxybenzoic acid) | BA | 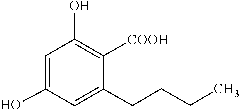 |
| Olivetolic acid (2,4-dihydroxy-6- pentylbenzoic acid) | OA | 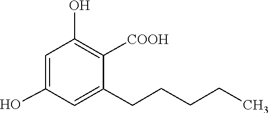 [SMILES: CCCCCc1cc(O)cc(O)c1C(=O)O] |
| 2-hexyl-4,6- dihydroxybenzoic acid | DHBA | 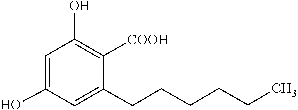 [SMILES: CCCCCCc1cc(O)cc(O)c1C(=O)O] |
| Sphaerophorolic acid (2-heptyl-4,6- dihydroxybenzoic acid) | PA | 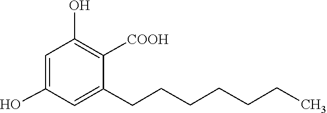 [SMILES: CCCCCCCc1cc(O)cc(O)c1C(=O)O] |

“Conversion” as used herein refers to the enzymatic conversion of a substrate(s) to a corresponding product(s). “Percent conversion” refers to the percent of the substrate that is converted to the product within a period of time under specified conditions. Thus, the “enzymatic activity” or “activity” of an enzymatic conversion can be expressed as “percent conversion” of the substrate to the product.

“Substrate” as used herein in the context of an enzyme mediated process refers to the compound or molecule acted on by the enzyme.

“Product” as used herein in the context of an enzyme mediated process refers to the compound or molecule resulting from the activity of the enzyme.

“Host cell” as used herein refers to a cell capable of being functionally modified with recombinant nucleic acids and functioning to express recombinant products, including polypeptides and compounds produced by activity of the polypeptides.

“Nucleic acid,” or “polynucleotide” as used herein interchangeably to refer to two or more nucleosides that are covalently linked together. The nucleic acid may be wholly comprised ribonucleosides (e.g., RNA), wholly comprised of 2′-deoxyribonucleotides (e.g., DNA) or mixtures of ribo- and 2′-deoxyribonucleosides. The nucleoside units of the nucleic acid can be linked together via phosphodiester linkages (e.g., as in naturally occurring nucleic acids), or the nucleic acid can include one or more non-natural linkages (e.g., phosphorothioester linkage). Nucleic acid or polynucleotide is intended to include single-stranded or double-stranded molecules, or molecules having both single-stranded regions and double-stranded regions. Nucleic acid or polynucleotide is intended to include molecules composed of the naturally occurring nucleobases (i.e., adenine, guanine, uracil, thymine, and cytosine), or molecules comprising that include one or more modified and/or synthetic nucleobases, such as, for example, inosine, xanthine, hypoxanthine, etc.

“Protein,” “polypeptide,” and “peptide” are used herein interchangeably to denote a polymer of at least two amino acids covalently linked by an amide bond, regardless of length or post-translational modification (e.g., glycosylation, phosphorylation, lipidation, myristilation, ubiquitination, etc.). As used herein “protein” or “polypeptide” or “peptide” polymer can include D- and L-amino acids, and mixtures of D- and L-amino acids.

“Naturally-occurring” or “wild-type” as used herein refers to the form as found in nature. For example, a naturally occurring nucleic acid sequence is the sequence present in an organism that can be isolated from a source in nature and which has not been intentionally modified by human manipulation.

“Recombinant,” “engineered,” or “non-naturally occurring” when used herein with reference to, e.g., a cell, nucleic acid, or polypeptide, refers to a material, or a material corresponding to the natural or native form of the material, that has been modified in a manner that would not otherwise exist in nature, or is identical thereto but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Nucleic acid derived from” as used herein refers to a nucleic acid having a sequence at least substantially identical to a sequence of found in naturally in an organism. For example, cDNA molecules prepared by reverse transcription of mRNA isolated from an organism, or nucleic acid molecules prepared synthetically to have a sequence at least substantially identical to, or which hybridizes to a sequence at least substantially identical to a nucleic sequence found in an organism.

“Coding sequence” refers to that portion of a nucleic acid (e.g., a gene) that encodes an amino acid sequence of a protein.

“Heterologous nucleic acid” as used herein refers to any polynucleotide that is introduced into a host cell by laboratory techniques, and includes polynucleotides that are removed from a host cell, subjected to laboratory manipulation, and then reintroduced into a host cell.

“Codon degenerate” describes a nucleotide sequence that has one or more different codons relative to the reference nucleotide sequence but which encodes a polypeptide that is identical to the polypeptide encoded by a reference nucleotide sequence. The different codons between the nucleotide sequence and the reference nucleotide sequence are called “synonyms” or “synonymous” codons in that they use different triplets of nucleotides to encode the same amino acid in a polypeptide.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several different “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the imine reductase enzymes may be codon optimized for optimal production from the host organism selected for expression.

“Preferred, optimal, high codon usage bias codons” refers to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; CodonW, John Peden, University of Nottingham; McInerney, J. O, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Control sequence” as used herein refers to all sequences, which are necessary or advantageous for the expression of a polynucleotide and/or polypeptide as used in the present disclosure. Each control sequence may be native or foreign to the nucleic acid sequence encoding a polypeptide. Such control sequences include, but are not limited to, a leader, a promoter, a polyadenylation sequence, a pro-peptide sequence, a signal peptide sequence, and a transcription terminator. At a minimum, control sequences typically include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” as used herein refers to a configuration in which a control sequence is appropriately placed (e.g., in a functional relationship) at a position relative to a polynucleotide sequence or polypeptide sequence of interest such that the control sequence directs or regulates the expression of the sequence of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Percentage of sequence identity,” “percent sequence identity,” “percentage homology,” or “percent homology” are used interchangeably herein to refer to values quantifying comparisons of the sequences of polynucleotides or polypeptides, and are determined by comparing two optimally aligned sequences over a comparison window, wherein the portion of the polynucleotide or polypeptide sequence in the comparison window may comprise additions or deletions (or gaps) as compared to the reference sequence for optimal alignment of the two sequences. The percentage values may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys, Madison Wis.), using default parameters provided.

“Reference sequence” refers to a defined sequence used as a basis for a sequence comparison. A reference sequence may be a subset of a larger sequence, for example, a segment of a full-length nucleic acid or polypeptide sequence. A reference sequence typically is at least 20 nucleotide or amino acid residue units in length, but can also be the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (or gaps) of 20 percent or less as compared to the reference sequence (which does not comprise additions or deletions) for optimal alignment of the two sequences.

“Substantial identity” or “substantially identical” refers to a polynucleotide or polypeptide sequence that has at least 70% sequence identity, at least 80% sequence identity, at least 85% sequence identity, at least 90% sequence identity, at least 95% sequence identity, or at least 99% sequence identity, as compared to a reference sequence over a comparison window of at least 20 nucleoside or amino acid residue positions, frequently over a window of at least 30-50 positions, wherein the percentage of sequence identity is calculated by comparing the reference sequence to a sequence that includes deletions or additions which total 20 percent or less of the reference sequence over the window of comparison.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered imine reductase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Isolated” as used herein in reference to a molecule means that the molecule (e.g., cannabinoid, polynucleotide, polypeptide) is substantially separated from other compounds that naturally accompany it, e.g., protein, lipids, and polynucleotides. The term embraces nucleic acids which have been removed or purified from their naturally-occurring environment or expression system (e.g., host cell or in vitro synthesis).

“Substantially pure” refers to a composition in which a desired molecule is the predominant species present (i.e., on a molar or weight basis it is more abundant than any other individual macromolecular species in the composition), and is generally a substantially purified composition when the object species comprises at least about 50 percent of the macromolecular species present by mole or % weight.

“Recovered” as used herein in relation to an enzyme, protein, or cannabinoid compound, refers to a more or less pure form of the enzyme, protein, or cannabinoid.

Recombinant Polypeptides with Enhanced Prenyltransferase Activity

The present disclosure provides engineered genes that encode recombinant polypeptides having prenyltransferase activity. When integrated into a recombinant host cell (e.g., S. cerevisiae) having a pathway capable of producing a cannabinoid precursor, such as olivetolic acid (OA), the presence of the engineered genes expressing the recombinant polypeptides results in an increased yield of the prenylated product of the cannabinoid precursor. In the case of a recombinant host cell capable of producing the cannabinoid precursor, OA, the prenylated product cannabinoid, CBGA, is produced by the host cell in greater yield relative to a comparable recombinant host cell integrated with the Cannabis sativa CsdPT4 prenyltransferase, which corresponds to the polypeptide of SEQ ID NO: 20. The enzymatic reaction step in the cannabinoid pathway of C. sativa catalyzed by the CsdPT4 polypeptide is the prenylation of the aromatic cannabinoid precursor substrate, OA (compound (2)) with the prenyl group donor substrate, GPP, to form the cannabinoid product CBGA (compound (1)), as shown in Scheme 1.

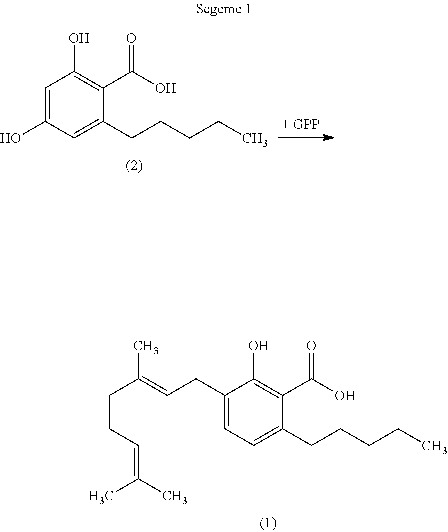 [SMILES: CCCCCc1cc(O)cc(O)c1C(=O)O ; CCCCCc1ccc(C/C=C(\C)CCC=C(C)C)c(O)c1C(=O)O]

The recombinant polypeptides with prenyltransferase activity of the present disclosure when incorporated in a recombinant host cell comprising a pathway that produces a cannabinoid precursor, such as OA (compound (2)), are capable, in the presence of GPP, of prenylating that substrate to form a cannabinoid product, such as CBGA (compound (1)). Without intending to be bound by any particular theory or mechanism, the conversion of the cannabinoid precursor substrate, OA (compound (2)), to the CBGA product (compound (1)) as in Scheme 1, when carried out by the recombinant polypeptides with prenyltransferase activity of the present disclosure integrated in a recombinant host cell results in a greater yield of the CBGA, relative to a control recombinant host cell strain integrated with a pathway that instead expresses the CsdPT4 polypeptide of SEQ ID NO: 20. The enhanced yield of the prenylated cannabinoid product is correlated with one or more residue differences in recombinant polypeptides of the present disclosure, as compared to the CsdPT4 amino acid sequence of SEQ ID NO:20, and/or correlated with codon differences in the nucleotide sequences encoding the polypeptides, as compared to the recombinant nucleic acid sequence of SEQ ID NO: 19. Exemplary engineered genes and encoded recombinant polypeptides with prenyltransferase activity that exhibit the unexpected and surprising technical effect of increased cannabinoid product yield when integrated in a recombinant host cell are summarized in Table 3 below.

TABLE 3
Recombinant polypeptides with prenyltransferase activity

| | NT SEQ | AA SEQ |
|---|---|---|
| aa difference and/or nt codon difference relative to CsdPT4 | ID NO: | ID NO: |
| n/a (CsdPT4) | 19 | 20 |
| F134G (TTT > GGG), S175V (TCT > GTG) | 21 | 22 |
| I79C | 23 | 24 |
| E106R (GAA > CGG), A182 (GCA > GCC) | 25 | 26 |
| W61A | 27 | 28 |
| S175V (TCT > GTT) | 29 | 30 |
| G58S, F73 (TTT > TTC) | 31 | 32 |
| W61V | 33 | 34 |
| F64M | 35 | 36 |
| F64L | 37 | 38 |
| F134G (TTT > GGT) | 39 | 40 |
| I79A (ATC > GCT) | 41 | 42 |
| S177A | 43 | 44 |
| F1731 | 45 | 46 |
| W153L (TGG > TTG) | 47 | 48 |
| F64G | 49 | 50 |
| I79S | 51 | 52 |
| G119 (GGG > GGT) | 53 | 54 |
| R152 (AGA > CGT) | 55 | 56 |
| G139 (GGT > GGG), S175V (TCT > GTG) | 57 | 58 |
| M80V | 59 | 60 |
| I79A (ATC > GCG) | 61 | 62 |
| S181R | 63 | 64 |
| S177G (TCA > GGT) | 65 | 66 |
| I113W | 67 | 68 |
| F134G (TTT > GGG) | 69 | 70 |
| E106R (GAA > CGG) | 71 | 72 |
| W153L (TGG > CTG) | 73 | 74 |
| T180R | 75 | 76 |
| S175T | 77 | 78 |
| R46K, F64T | 79 | 80 |
| S177G (TCA > GGG) | 81 | 82 |
| F132 (TTC > TTT) | 83 | 84 |
| I165L | 85 | 86 |
| T180V | 87 | 88 |
| F75W | 89 | 90 |
| S177T | 91 | 92 |
| T180L | 93 | 94 |
| A293G | 95 | 96 |
| N235C | 97 | 98 |
| F161V (TTC > GTT), A293V | 99 | 100 |
| F158G (TTT > GGG) | 101 | 102 |
| S295A | 103 | 104 |
| E284R (GAA > CGG) | 105 | 106 |
| N50D, E284R (GAA > AGG) | 107 | 108 |
| V99A (GTT > GCG) | 109 | 110 |
| E284D | 111 | 112 |
| E284K (GAA > AAA), A291E | 113 | 114 |
| P294E | 115 | 116 |
| E284R (GAA > AGG) | 117 | 118 |
| Q82 (CAA > CAG) | 119 | 120 |
| A293K | 121 | 122 |
| D87E, V99A (GTT > GCG) | 123 | 124 |
| N235V | 125 | 126 |
| P97 (CCA > CCG) | 127 | 128 |
| F161V (TTC > GTT) | 129 | 130 |
| F158G (TTT > GGG) | 131 | 132 |
| E284K (GAA > AAG) | 133 | 134 |
| T229V | 135 | 136 |
| N235K | 137 | 138 |
| P5G, H7C, C41S, F64T, F134G, S175V, S177A, G204S, L249A, | 139 | 140 |
| S295A | | |
| H7C, D10V, C41A, R46K, F64T, I79C, K125W, F134G | 141 | 142 |
| H7C, D10V, I79C, F134G, S175T, S177A, T180R, R190S, G204S | 143 | 144 |
| P5G, H7C, D10V, T180R, G204S, S241F | 145 | 146 |
| D10V, C41S, F64T, I79C, W153L, S175T, T180R, G204S, L249A, | 147 | 148 |
| C277M, F280R, Q281R, A291E, S295A, Y307H, A308E, E309V, | | |
| Y310S | | |
| P5G, H7C, C41A, F64T, I79A, I113N, W153L, S175V, T180R, S194L, | 149 | 150 |
| I197T, G204S | | |
| P5G, H7C, D10V, F64T, W153L, S175V, V188A, R190S | 151 | 152 |
| H7C, R46K, I79C, K125W, F134G, Y176S, S177A, T180R, G204S, | 153 | 154 |
| C277A, L278P, F280G, Q281R, T282P | | |
| H7C, C41S, R46K, I79C, K125W, S175T, S177T, T180R, R190S, | 155 | 156 |
| G204S, S251A, C277M, Q281R, A291E | | |
| P5G, C41A, K125W, W153L, S175T, S177A, T180R | 157 | 158 |
| H7C, S177T, T180R, S194V, G204A, S295A | 159 | 160 |
| P5G, H7C, C41A, F64T, K125W, F134G, S177A, G204S, C277A, | 161 | 162 |
| F280R, F301S | | |
| D10V, C41S, R46K, F134G, W153L, S177T, T180R, V188A, R190S, | 163 | 164 |
| M205G, L249A, C277M, F280R | | |
| P5G, H7C, D10V, F49L, R52P, K125W, W153L, S175V, S177T, | 165 | 166 |
| T180R, S194L, G204S, M205G | | |
| H7C, D10V, C41A, R46K, R52P, S175V, S177A, T180R, V188A, | 167 | 168 |
| G204S, M205G | | |
| P5G, H7C, I79C, F134G, W153L, S175V | 169 | 170 |
| D10V, C41S, R46K, F134G, W153L, S175V, G204S | 171 | 172 |
| H7C, R46K, I79A, S177A, T180R, V188A, G204S | 173 | 174 |
| H7C, D10V, C41A, K125W, W153L, S175T, V188S, R190S, M200R, | 175 | 176 |
| M205G, S214C, D219V, V243A, S251C, S264Y, Q281R, A288P | | |
| H7C, K125W, S175V, S177T, T180R, S194L, G204S, S251A, S295A | 177 | 178 |
| H7C, D10V, C41A, R46K, V68D, I79A, W153L, S175V, S177T, | 179 | 180 |
| T180R, V188A, R190S, G204S, M205G | | |
| P5G, R46K, R52P, F64T, L249A, E284R, A291E, S295A | 181 | 182 |
| P5G, H7C, C41S, R46K, K125W, F134G, I165T, S175T, S177T, | 183 | 184 |
| T180R, G204S, Q281R, S295A | | |
| H7C, D10V, C41A, F64T, W153L, S175V, S177A, T180R, M205G | 185 | 186 |
| P5G, C41S, R52P, I79C | 187 | 188 |
| P5G, H7C, R52P, I79A, F134G, W153L, G204S, M205G, L249A, | 189 | 190 |
| C277A, F280R, Q281R, A291E | | |
| D10V, R46K, W153L, T180R, S194V, L249A, C277M, F280R, Q281R, | 191 | 192 |
| A291E, S295A | | |
| D10V, C41S, K125W, F134G, S175V, S177A, T180R | 193 | 194 |
| P5G, D10V, C41S, K125W, F134G, T180R | 195 | 196 |
| H7C, I79A, K125W, W153L, S175T, T180R, R190S, M205G, L249A, | 197 | 198 |
| S251A, C277A, F280R, A291E | | |
| P5G, H7C, D10V, C41S, R46K, I121T, K125W, F134G, W153L, | 199 | 200 |
| S175V, S177T, T180R, V188A, R190S, M205G | | |
| P5G, C41S, S175V, S177A, T180R, L249A, C277A | 201 | 202 |
| H7C, D10V, F49L, I79C, W153L, S175V, S177T, T180R, V188A, | 203 | 204 |
| S194L | | |
| P5G, H7C, C41S, F49L, R52P, F64T, I79C, K125W, W153L, S175T, | 205 | 206 |
| S177T, T180R, V188A, R190S, G204S, C277M, A291E | | |
| P5G, H7C, D10V, F64T, F134G, W153L, S177T, T180R, L249A, | 207 | 208 |
| C277A, Q281R, A291E, S295A | | |
| H7C, D10V, I79C, S177A, T180R, V188A, R190S, A291E, S295A | 209 | 210 |
| H7C, D10V, C41S, R46K, I79A, K125W, S175V, T180R, R190S | 211 | 212 |
| P5G, H7C, D10V, I79C, K125W, S175V, S177T, R190S, G204S, | 213 | 214 |
| M205G, L249A, S251A, C277M, A291E | | |
| D10V, R46K, K125W, W153L, S175V, S177A, T180R, V188A, | 215 | 216 |
| C277M, S295A | | |
| P5G, H7C, C41S, F64T, K125W, F134G, S175V, S177A, V188A, | 217 | 218 |
| M205G | | |
| P5G, C41A, R46K, F134G, S175T, T180R, V188A, R190S | 219 | 220 |
| H7C, D10V, C41A, R52P, K125W, F134G, S177T, S194V | 221 | 222 |
| P5G, D10V, R52P, I79C, K125W, W153L | 223 | 224 |
| P5G, H7C, D10V, F49L, K125W, F134G, W153L, S194L, G204S, | 225 | 226 |
| Q281R, S295A | | |
| P5G, R52P, F64T, I79C, D95N | 227 | 228 |
| P5G, H7C, I79A, I105V, S177A, T180R, S194V, M205G, Q281R, | 229 | 230 |
| S295A, V314A | | |
| D10V, C41S, R46K, R52P, F64T, F134G, S177A, T180R | 231 | 232 |
| P5G, D10V, R46K, R52P, F64T, I79C, K125W, W153L, T180R | 233 | 234 |
| H7C, C41S, R52P, I79C, K125W, F134G, W153L, S175T, T180I, | 235 | 236 |
| V188A, R190S, L249A, S251A, F280R, Q281R, S295A | | |
| H7C, D10V, K125W, F134G, W153L, S175T, S177T | 237 | 238 |
| P5G, C41S, R46K, K125W, F134G, W153L, S175T, S177A, S194V | 239 | 240 |
| P5G, D10V, I79A, K125W, F134G, S175V, C277M, Q281R, A291E, | 241 | 242 |
| S295A | | |
| P5G, D10V, C41A, R46K, R52P, F134G, W153L, S175T, S177T, | 243 | 244 |
| M205G, L249A, S251A, F280R, Q281R, A291E | | |
| P5G, C41S, I79C, W153L, S175T, M205G, L249A, S251A, C277A, | 245 | 246 |
| F280R, A291E, Y313P | | |
| P5G, H7C, D10V, C41A, R52P, T123K, K125W | 247 | 248 |
| P5G, D10V, C41A, R46K, K125W, F134G, W153L, S175V, S177T, | 249 | 250 |
| T180R, G204S, M205G, S253P, F280R, A291E, S295A, F315S | | |
| H7C, C41A, F49L, I79C, K125W, W153L, S177T | 251 | 252 |
| P5G, H7C, C41A, S177A, T180R, M205G, L249A, S251A, C277A, | 253 | 254 |
| A291E, S295A, F299L | | |
| H7C, D10V, C41S, F64T, S177A, V188A, M205G, L249A, S251A, | 255 | 256 |
| A291E, S295A | | |
| H7C, D10V, R46K, F134V | 257 | 258 |
| P5G, H7C, F49L, F64T, I79C, K125W, W153L, S175T, S194L, | 259 | 260 |
| C277M, F280R, Q281R, A291E, S295A | | |
| P5G, D10V, F64T, F134G, S175T, S177T, T180R, L249A, S251A, | 261 | 262 |
| A291E | | |
| P5G, H7C, D10V, I79A, K125W, W153L, E284K, A291E | 263 | 264 |
| P5G, D10V, F49L, R52P, F134G, S194L, S251A, W258R, Q281R, | 265 | 266 |
| A291E, S295A | | |
| H7C, D10V, C41S, I79C, F134G, V188A, R190S, S214C, A291E, | 267 | 268 |
| S295A, F311S | | |
| C41A, I79A, K125W, F134G, W153L, S175T, S177T, T180R | 269 | 270 |
| P5G, H7C, I79A, S175V, S177T, L249A, S251A, W258R, Q281R | 271 | 272 |
| H7C, D10V, C41A, R46K, F64T, V115A, K125W, T180R, C277A, | 273 | 274 |
| F280R, S295A | | |
| D10V, C41A, R46K, R52P, F64T, K125W, F134G, W153L, S177A, | 275 | 276 |
| T180R, S214C, L249A, E284K, S295A, A308P | | |
| P5G, D10V, C41A, R46K, I79A, F134G, W153L, L249A, Q281R, | 277 | 278 |
| S295A | | |
| P5G, H7C, R46K, K125W, S175V, S177T, T180R, M205G, L249A, | 279 | 280 |
| E284K, S295A | | |
| D10V, C41S, R46K, F65L, K125W, W153L, S177A, T180R, S194L, | 281 | 282 |
| L249A, S251A, C277A | | |
| C41A, R46K, I79C, K93N, K125W | 283 | 284 |
| P5G, H7C, R52P, I79C, K125W, W153L, S177T, T180R, S194L | 285 | 286 |
| C41A, R46K, W153L, S175V, M205G | 287 | 288 |
| H7C, R52P, W153L, S175V, G204S, M205G, C277M, Q281R, S295A | 289 | 290 |
| P5G, R52P, I79A, K125W | 291 | 292 |
| P5G, H7C, D10V, C41S, F49L, R52P, F64T, S175V, S177A, M205G | 293 | 294 |
| P5G, F49L, I79A, S177T | 295 | 296 |
| P5G, D10V, C41S, R46K, I79N, F134G, S175V, S177T, T180R, | 297 | 298 |
| M205G, A291E, S295A | | |
| H7C, D10V, R46K, K125W, F158S, S175V, S177T, T180R, V188A, | 299 | 300 |
| R190S, M205R, L249A, C277R, F280R, Q281R, A291E, Y310S | | |
| P5G, H7C, C41A, R52P, F134G, W153L, S175T, L249A, S251A, | 301 | 302 |
| E284R, S295A, L305S | | |
| H7C, C41S, R46K, K125W, S194L, Q281R, A291E | 303 | 304 |
| H7C, F49L, K125W, F134G, S177T, M205G, E284R, A291E, S295A | 305 | 306 |
| P5G, H7C, D10V, C41A, R46K, F134G, F144S, W153L, G204S, | 307 | 308 |
| L249A, S251A, C277M, F280R, Q281R | | |
| P5G, R46K, L54S, K125W, W153L, S175T, S177T, S214C, F276L, | 309 | 310 |
| F280R, Q281R, A308P, Y310C, Y313H | | |
| P5G, D10V, R46K, I79C | 311 | 312 |
| P5G, D10V, R46K, W153L, S175V, S177T, T180R, S214C, S251A, | 313 | 314 |
| C277M, F280R, Q281R, S295A, F301S, I302L, W303C, L304R, | | |
| Y310S, F311S | | |
| D10V, R46K, F64T, I79A, W153L, S177A, T180R, S194L, S251A, | 315 | 316 |
| S295A | | |
| H7C, D10V, C41A, S175V, F193L | 317 | 318 |
| H7C, C41A, R46K, R52P, K125W, F134G, S177T | 319 | 320 |
| P5G, H7C, D10V, C41A, K125W, S194L, G204S, M205G, F280R, | 321 | 322 |
| A291E, S295A | | |
| P5G, F49L, R52P, K125W, F134G, W153L, S177T, R190S, M205G, | 323 | 324 |
| S214C, F280R, A291E, S295A, V312G, Y313H | | |
| H7C, D10V, K125W, F134G | 325 | 326 |
| D10V, F49L, R52P, F64T, W153L, S175V, S177A, Q281R, S295A, | 327 | 328 |
| F311P | | |
| C41S | 329 | 330 |
| H7C, D10V, C41S, R46K, K125W, W153L, S194A | 331 | 332 |
| R52P, F64T, I79C, F134G, S177A, T180R, L249A, M267T, C277M, | 333 | 334 |
| Q281R, L287F, A288P, Y290S | | |
| D10V, F64T, I79C, K125W, F134G, I140T, S177A, L249A, C277M, | 335 | 336 |
| Q281R, A291E | | |
| D10V, K34E, F49L | 337 | 338 |
| H7C, D10V, C41A, R46K, F64T, K125W, R190S, M205G, C277A, | 339 | 340 |
| S295A, Y307S, A308R, Y310S | | |
| P5G, H7C, D10V, C41G, K125W, F134G, L249V, F280L, A291E | 341 | 342 |
| P5G, F64T, I79C, W153L, I165T, Q281R, A291E, S295A | 343 | 344 |
| H7C, R46K, F64T, I91V, W153L, S175V, S177T, I196T, M205G, | 345 | 346 |
| L249A, C277M, A291E, Y310P | | |
| H7C, C41A, K125W, F134G, W153L, S175T, T180R, R190S, M205G, | 347 | 348 |
| E217G | | |
| P5G, D10W, C41G, F49M, W61A, F64W, I79A, K125M, F158G, | 349 | 350 |
| S175A, S177A, T180L, R190S, S194V, N235K, F238W, C277A, | | |
| E284D, A293V | | |
| P5G, D10V, C41S, F49M, W61A, F64L, I79C, K125V, F158G, S175V, | 351 | 352 |
| S177A, T180V, R190S, S194V, N235C, F238L, C277A, E284K, | | |
| A293K | | |
| P5G, D10V, C41A, F49L, W61A, F64G, I79C, K125V, F158G, S175A, | 353 | 354 |
| S177T, T180V, R190S, S194V, N235K, F238W, C277A, E284D, | | |
| A293G | | |
| C41A, F49L, W61V, F64T, I79A, K125M, F158G, S175A, S177T, | 355 | 356 |
| T180L, R190A, S194V, N235C, F238W, C277A, E284D, A293G | | |
| P5G, D10L, C41A, F49L, W61A, F64T, I79C, K125M, F158G, S175A, | 357 | 358 |
| S177T, T180R, R190S, S194A, N235K, F238W, C277M, E284K, | | |
| A293V | | |
| P5G, D10W, C41S, F49L, W61A, F64T, I79A, K125M, F158G, S175A, | 359 | 360 |
| S177A, T180L, R190Q, S194L, N235C, F238L, C277M, E284K, | | |
| A293V | | |
| P5G, D10L, C41G, F49R, W61A, F64T, I79C, K125W, F158G, S175T, | 361 | 362 |
| S177T, T180R, R190S, S194L, N235K, F238L, C277A, E284D, | | |
| A293G | | |
| P5V, D10L, C41A, F49L, W61A, F64W, I79A, K125V, F158G, S175V, | 363 | 364 |
| S177T, T180R, R190A, S194A, N235C, F238L, C277A, E284R, | | |
| A293G | | |
| P5G, D10V, C41A, F49L, W61A, F64M, I79C, K125V, F158G, S175A, | 365 | 366 |
| S177T, T180L, R190S, S194V, N235C, F238W, C277M, E284R, | | |
| A293G | | |
| P5G, D10W, C41A, F49L, W61A, F64G, I79C, K125W, F158G, | 367 | 368 |
| S175T, S177T, T180R, R190S, S194L, N235C, F238W, C277A, | | |
| E284K, A293G | | |
| P5V, D10L, C41A, F49L, W61V, F64M, I79C, K125V, F158G, S175V, | 369 | 370 |
| S177T, T180R, R190A, S194A, N235C, F238W, C277M, E284K, | | |
| A293V | | |
| P5G, D10L, N11D, C41G, F49L, W61V, F64W, I79C, K125M, F158G, | 371 | 372 |
| S175A, S177T, T180V, R190A, S194A, N235C, F238L, C277M, | | |
| E284K, A293G | | |
| P5G, D10V, C41A, F49R, W61A, F64M, I79C, K125W, F158G, | 373 | 374 |
| S175A, S177A, T180R | | |
| P5V, D10V, C41S, F49M, W61A, F64M, I79A, K125W, F158G, | 375 | 376 |
| S175A, S177G, T180R, R190S, S194V, N235K, F238L, C277M, | | |
| E284R, A293G | | |
| P5V, D10V, C41A, F49R, W61A, F64L, I79A, K125M, F158G, S175T, | 377 | 378 |
| S177G, T180V, R190Q, S194A, N235C, F238L, C277M, E284K, | | |
| A293V | | |
| P5G, D10V, C41A, F49L, W61V, F64G, I79A, K125V, A129T, F158G, | 379 | 380 |
| S175V, S177A, T180R, R190Q, S194L, N235V, F238L, C277M, | | |
| E284D, A293G | | |
| P5G, D10V, C41A, F49L, W61V, F64L, I79A, K125V, F158G, S175A, | 38 | 382 |
| S177T, T180L, R190A, S194A, N11D, N235C, F238L, C277M, | | |
| E284D, A293G | | |
| P5V, D10V, C41A, F49L, W61V, F64T, I79C, K125M, F158G, S175A, | 383 | 384 |
| S177A, T180L, R190Q, S194A, N235V, F238W, C277M, E284K, | | |
| A293G | | |
| P5V, D10W, C41S, F49M, W61A, F64L, I79A, K125W, F158G, | 385 | 386 |
| S175A, S177T, T180V, R190S, S194A, N235K, F238L, C277A, | | |
| E284D, A293K | | |
| P5V, D10L, C41A, F49L, W61A, F64M, I79C, K125M, F158G, S175A, | 387 | 388 |
| S177A, T180L, R190S, S194L, N235C, F238L, C277A, E284R, | | |
| A293G | | |
| P5G, D10V, C41S, F49L, W61V, F64L, I79A, K125W, F158G, S175A, | 389 | 390 |
| S177G, T180R, R190S, S194A, N235C, F238L, C277M, E284R, | | |
| A293G | | |
| P5V, D10V, C41A, F49L, W61V, F64M, I79A, K125W, F158G, S175A, | 391 | 392 |
| S177G, T180V, R190G, S194A, N235C, F238W, C277A, E284R, | | |
| A293V | | |
| P5V, D10V, C41S, F49M, W61V, F64G, I79C, K125W, F158A, S175T, | 393 | 394 |
| S177G, T180V, R190S, S194A, N235K, F238W, C277A, E284D, | | |
| A293G | | |
| P5V, D10W, C41S, F49L, W61A, F64L, I79C, K125M, F138I, F158G, | 395 | 396 |
| S175A, S177A, T180L, R190S, S194V, N235K, F238W, C277M, | | |
| E284R, A293G | | |
| C41S, F49R, W61A, F64L, I79C, K125V, F158A, S175V, S177A, | 397 | 398 |
| T180R, R190S, S194L, N235C, F238W, C277A, E284D, A293G | | |
| P5G, D10V, C41A, F49M, W61A, F64G, I79A, K125W, F158G, | 399 | 400 |
| S175G, S177T, T180R, R190S, S194V, N235K, F238L, C277A, | | |
| E284R, A293G | | |
| P5G, D10V, C41S, F49L, W61A, F64G, I79A, K125W, F158G, S175A, | 401 | 402 |
| S177G, T180L, R190Q, S194V, N235C, F238W, C277M, E284K, | | |
| A293G | | |
| P5G, D10V, C41S, F49M, W61A, F64M, I79A, K125W, F158A, | 403 | 404 |
| S175A, S177T, T180V, R190S, S194L, N235C, F238L, C277M, | | |
| E284D, A293K | | |
| P5G, D10V, C41A, F49M, W61A, F64L, I79C, K125M, A129T, F158G, | 405 | 406 |
| S175G, S177A, T180R, R190A, S194L, N235K, F238L, C277A, | | |
| E284D, A293G | | |
| P5G, D10L, C41S, F49M, W61V, F64W, I79C, K125V, F158A, S175T, | 407 | 408 |
| S177T, T180R, R190S, S194A, N235K, F238L, C277A, E284R, | | |
| A293G | | |
| P5V, D10W, C41G, F49R, W61V, F64T, I79A, K125V, F158G, S175T, | 409 | 410 |
| S177A, T180V, R190S, S194V, N235K, F238L, C277M, E284D, | | |
| A293G | | |
| P5G, D10V, C41A, F49L, W61A, F64M, I79C, K125M, F158G, S175V, | 411 | 412 |
| S177A, T180V, R190Q, S194A, N235V, F238W, C277M, E284D, | | |
| A293G | | |
| P5V, D10W, C41S, F49M, W61A, F64T, I79C, K125M, F158G, | 413 | 414 |
| S175V, S177T, T180V, R190A, S194A, N235K, F238W, C277A, | | |
| E284D, A293K | | |
| C41A, F49L, W61A, F64T, I79A, K125M, F158A, S175V, S177G, | 415 | 416 |
| T180R, R190Q, S194A, N235C, F238W, C277M, E284D, A293G | | |
| P5G, D10L, C41S, F49M, W61V, F64M, I79C, K125W, F158A, | 417 | 418 |
| S175A, S177G, T180R, R190S, S194A, N235K, F238W, C277M, | | |
| E284D, A293G | | |
| P5V, D10W, C41G, F49R, W61A, F64M, I79A, K125V, F158A, S175V, | 419 | 420 |
| S177T, T180R, R190S, S194V, N235C, F238L, C277M, E284R, | | |
| A293K | | |
| P5V, D10V, C41A, F49L, W61V, F64M, I79A, K125W, F158A, S175A, | 421 | 422 |
| S177G, T180R, R190S, S194V, N235C, F238L, C277A, E284D, | | |
| A293G | | |
| P5G, D10W, C41G, F49L, W61V, F64W, I79C, K125W, F158G, | 423 | 424 |
| S175A, S177T, T180V, R190A, S194A, N235V, F238W, C277M, | | |
| E284K, A293K | | |
| P5G, D10V, C41S, F49M, W61V, F64W, I79A, K125W, F158G, | 425 | 426 |
| S175V, S177T, T180R, R190Q, S194A, N235C, F238L, C277A, | | |
| E284D, A293K | | |
| P5G, D10L, C41G, F49L, W61A, F64M, I79A, K125V, F158A, S175A, | 427 | 428 |
| S177G, T180V, R190G, S194L, N235V, F238W, C277M, E284R, | | |
| A286G, A293G | | |
| P5G, D10W, C41A, F49L, W61A, F64M, I79A, K125W, F158G, | 429 | 430 |
| S175G, S177T, T180L, R190Q, S194A, N235K, F238L, C277A, | | |
| E284R, A293G | | |
| P5V, D10L, C41S, F49L, W61A, F64L, I79C, K125M, F158A, S175G, | 431 | 432 |
| S177A, T180R, R190Q, S194V, N235C, F238L, C277M, E284R, | | |
| A293G | | |
| C41S, F49L, W61A, F64M, I79A, K125M, F158A, S175V, S177T, | 433 | 434 |
| T180R, R190G, S194L, N235V, F238L, C277A, E284D, A293G | | |
| P5G, D10V, C41A, F49M, W61A, F64T, I79C, K125V, F158A, S175V, | 435 | 436 |
| S177A, T180L, R190S, S194A, N235V, F238W, C277M, E284R, | | |
| A293G | | |
| P5G, D10V, C41G, F49R, W61A, F64T, I79A, K125W, F158A, S175G, | 437 | 438 |
| S177T, T180L, R190S, S194L, N235C, F238L, C277M, E284D, | | |
| A293G | | |
| P5G, D10W, C41A, F49M, W61V, F64T, I79C, K125M, F158A, | 439 | 440 |
| S175T, S177A, T180L, R190A, S194L, N235C, F238W, C277M, | | |
| E284D, A293G | | |
| P5G, D10L, C41A, F49L, W61A, F64L, I79A, K125W, F158A, S175G, | 441 | 442 |
| S177T, T180V, R190A, S194A, N235C, F238W, C277M, E284R, | | |
| A293G | | |
| P5G, D10L, C41A, F49M, W61A, F64L, I79C, K125V, F158A, S175G, | 443 | 444 |
| S177A, T180V, R190A, F195V, S194L, N235C, F238W, C277A, | | |
| E284D, A293G | | |
| P5V, D10L, C41G, F49M, W61V, F64T, I79C, K125W, F158A, S175T, | 445 | 446 |
| S177A, T180V, R190S, S194V, N235C, F238W, C277A, E284D, | | |
| A293G | | |
| P5G, D10W, C41S, F49L, W61V, F64L, I79A, K125W, F158A, S175A, | 447 | 448 |
| S177G, T180R, R190G, S194L, N235C, F238W, C277A, E284R, | | |
| A293G | | |
| P5G, D10V, C41A, F49M, W61A, F64G, I79A, K125V, F158G, S175A, | 449 | 450 |
| S177A, T180L, R190Q, S194L, N235C, F238W, C277M, E284D, | | |
| A293V | | |
| P5G, D10W, C41G, F49M, W61A, F64W, I79C, K125M, F158G, | 451 | 452 |
| S175A, S177A, T180L, R190A, S194L, N235K, F238W, C277M, | | |
| E284D, A293G | | |
| P5V, D10W, C41G, F49L, W61A, F64W, I79C, K125V, F158G, | 453 | 454 |
| S175G, S177G, T180L, R190S, S194A, N235K, F238L, C277A, | | |
| E284D, A293V | | |
| P5G, D10W, C41A, F49L, W61A, F64G, I79C, K125W, F158A, | 455 | 456 |
| S175A, S177A, T180V, R190S, S194A, N235K, F238W, C277A, | | |
| E284R, A293V | | |
| P5V, D10L, C41S, F49L, W61A, F64M, I79A, K125W, F158A, S175A, | 457 | 458 |
| S177A, T180L, R190S, S194L, N235C, F238L, C277A, E284D, | | |
| A293G | | |
| P5G, D10L, C41A, F49L, W61A, F64M, I79C, K125V, F158A, S175V, | 459 | 460 |
| S177T, T180R, R190A, S194A, N235C, F238W, C277M, E284K, | | |
| A293G | | |
| P5G, D10V, C41S, F49L, W61A, F64T, I79C, K125M, F158A, S175T, | 461 | 462 |
| S177T, T180L, R190Q, S194L, N235C, F238L, C277A, E284D, | | |
| A293G | | |
| K125W, F158A, S175A, S177T, T180L, R190Q, S194A, N235C, | 463 | 464 |
| F238L, C277A, E284D, A293V | | |
| P5G, D10V, C41A, F49L, W61V, F64L, I79A, K125M, F158A, S175G, | 465 | 466 |
| S177G, T180L, R190G, S194V, N235K, F238L, C277A, E284R, | | |
| A293K | | |
| P5V, D10L, C41G, F49L, W61V, F64T, I79C, K125V, F158G, S175G, | 467 | 468 |
| S177T, T180R, R190S, S194L, N235C, F238L, C277M, E284R, | | |
| A293G | | |
| P5V, D10V, C41S, F49M, W61A, F64M, I79A, K125W, F158A, S175A, | 469 | 470 |
| S177G, T180L, R190G, S194A, N235C, F238L, C277A, E284D, | | |
| A293V | | |
| P5G, D10W, C41S, F49M, W61A, F64W, I79C, K125W, F158A, | 471 | 472 |
| S175A, S177A, T180R, R190S, S194A, N235C, F238W, C277M, | | |
| E284D, A293G | | |
| P5V, D10V, C41G, F49L, W61V, F64M, I79C, K125V, F158A, S175G, | 473 | 474 |
| S177T, T180L, R190Q, S194A, N235V, F238L, C277M, E284R, | | |
| A293G | | |
| P5V, D10L, C41S, F49L, W61A, F64T, I79C, K125M, F158G, S175G, | 475 | 476 |
| S177A, T180L, R190A, S194A, N235C, F238L, C277M, E284K, | | |
| A293G | | |
| P5G, D10V, C41A, F49L, W61A, F64G, I79C, K125W, F158A, S175A, | 477 | 478 |
| S177T, T180R, R190S, S194V, N235C, F238W, C277A, E284D, | | |
| A293G | | |
| P5G, D10W, C41G, F49R, W61A, F64L, I79C, K125M, F158A, | 479 | 480 |
| S175A, S177A, T180L, R190A, S194V, N235C, F238W, C277M, | | |
| E284D, A293V | | |
| C41A, F49L, W61V, F64L, I79A, K125V, F158G, S175G, S177G, | 481 | 482 |
| T180L, R190G, S194A, N235V, F238W, C277A, E284D, A293G | | |
| K125W, F158A, S175A, S177G, T180V, R190S, S194L, N235K, | 483 | 484 |
| F238W, C277M, E284R, A293G | | |
| P5V, D10V, C41S, F49M, W61A, F64M, I79C, K125M, F158A, | 485 | 486 |
| S175G, S177T, T180R, R190S, S194A, N235V, F238W, C277A, | | |
| E284R, A293G | | |
| P5G, D10W, C41A, F49L, W61A, F64L, I79C, K125W, F158A, S175A, | 487 | 488 |
| S177G, T180V, R190A, S194A, N235C, F238W, C277A, E284D, | | |
| A293G | | |
| P5G, D10W, C41S, F49L, W61A, F64W, I79A, K125V, F158A, S175T, | 489 | 490 |
| S177G, T180V, R190A, S194L, N235V, F238L, C277M, E284D, | | |
| A293V | | |
| P5V, D10W, C41G, F49L, W61A, F64G, I79A, K125M, F158A, | 491 | 492 |
| S175G, S177A, T180L, R190S, S194V, N235V, F238L, C277A, | | |
| E284D, A293K | | |
| P5G, D10L, C41A, F49L, W61A, F64W, I79C, K125W, F158A, S175V, | 493 | 494 |
| S177A, T180L, R190S, S194L, N235K, F238W, C277M, E284D, | | |
| A293G | | |
| P5V, D10L, C41G, F49R, W61V, F64M, I79A, K125W, F158A, S175T, | 495 | 496 |
| S177A, T180L, R190Q, S194L, N235C, F238W, C277M, E284K, | | |
| A293V | | |
| P5V, D10W, C41G, F49L, W61A, F64G, I79A, K125V, F158A, S175T, | 497 | 498 |
| S177G, T180V, R190Q, S194V, N235V, F238W, C277M, E284K, | | |
| A293G | | |
| P5V, D10W, C41G, F49R, W61A, F64L, I79A, K125M, F158G, | 499 | 500 |
| S175G, S177A, T180L, R190S, S194L, N235C, F238W, C277M, | | |
| E284D, A293G | | |
| P5V, D10W, C41G, F49R, W61A, F64T, I79A, K125V, F158A, S175A, | 501 | 502 |
| S177T, T180R, R190G, S194V, N235K, F238W, C277M, E284D, | | |
| A293G | | |
| P5G, D10W, C41S, F49L, W61A, F64T, I79C, K125V, F158A, S175G, | 503 | 504 |
| S177A, T180L, R190S, S194A, N235V, F238L, C277A, E284D, | | |
| A293G | | |
| P5V, D10W, C41G, F49L, W61A, F64M, I79A, K125V, F158A, S175G, | 505 | 506 |
| S177G, T180L, R190Q, S194L, N235C, F238L, C277A, E284D, | | |
| A293V | | |
| P5G, D10V, C41S, F49M, W61A, F64W, I79C, K125V, F158G, | 507 | 508 |
| S175G, S177T, T180R, R190A, S194L, N235C, F238L, C277M, | | |
| E284R, A293G | | |
| P5G, D10V, C41G, F49R, W61V, F64M, I79A, K125V, F158G, | 509 | 510 |
| S175G, S177G, T180V, R190S, S194V, N235K, F238W, C277A, | | |
| E284K, A293G | | |
| P5G, D10L, C41S, F49L, W61V, F64L, I79A, K125M, F158A, S175G, | 511 | 512 |
| S177G, T180V, R190A, S194A, N235K, F238W, C277M, E284D, | | |
| A293G | | |
| F49R, W61V, F64M, I79A, K125W, F158A, S175G, S177A, T180V, | 513 | 514 |
| R190Q, S194V, N235V, F238W, C277M, E284R, A293G | | |

In at least one embodiment, the recombinant polypeptides having prenyltransferase activity and increased activity have one or more residue differences as compared to the reference prenyltransferase polypeptide of SEQ ID NO: 20. In some embodiments, the recombinant polypeptides have one or more residue differences at residue positions selected from R46, N50, G58, W61, F64, F75, I79, M80, O87, V99, E106, I113, F134, W153, F158, F161, I165, F173, S175, S177, T180, S181, T229, N235, E284, A291, A293, P294, and S295. In at least one embodiment, the amino acid residue differences are: R46K, N50D, G58S, W61A, W61V, F64G, F64L, F64M, F64T, F75W, I179A, I79C, I79S, M80V, D87E, V99A, E106R, I113W, F134G, W153L, F158G, F161V, I165L, F173I, S175T, S175V, S177A, S177G, S177T, T180L, T180R, T180V, S181R, T229V, N235C, N235K, N235V, E284D, E284K, E284R, A291E, A293G, A293K, A293V, P294E, and S295A.

In at least one embodiment, the recombinant polypeptides having prenyltransferase activity and increased activity have one or more residue differences as compared to the reference prenyltransferase polypeptide of SEQ ID NO: 20. In some embodiments, the recombinant polypeptides have one or more residue differences at residue positions selected from W61, F64, I79, F134, W153, F158, S175, S177, T180, N235, E284, and A293. In at least one embodiment, the amino acid residue differences are selected from: W61A, W61V, F64G, F64L, F64M, F64T, F64W, I179A, I79C, I79N, I79S, F134G, F134V, W153L, F158A, F158G, F158S, S175A, S175G, S175T, S175V, Y176S, S177A, S177G, S177T, T180I, T180L, T180R, T180V, N235C, N235K, N235V, E284D, E284K, E284R, A293G, A293K, and A293V.

It is contemplated that the residue differences relative to SEQ ID NO: 20 at residue positions associated with increased prenyltransferase activity can be used in various combinations to form recombinant prenyltransferase polypeptides having desirable functional characteristics when integrated in a recombinant host cell, for example increased yield product of the cannabinoid product compound, CBGA. Some exemplary combinations of amino acid differences include those combinations found in the polypeptides of Table 3 and elsewhere herein. For example, the present disclosure provides a recombinant polypeptide having increased prenyltransferase activity and amino acid residue differences as compared to SEQ ID NO: 20 at various combinations of the following positions: W61, F64, I79, F134, W153, F158, S175, S177, T180, N235, E284, and A293. In at least one embodiment, the recombinant polypeptides can comprise a combination of amino acid differences selected from:

F64T, E284R
F64T, I79C
F64T, S177A
F64T, T180R
F64T, I79C, W153L
F64T, I79C, F134G
F64T, F134G, S177A
F64T, S175V, S177A
F64T, I79C, W153L, T180R
F64T, F134G, S175V, S177A
F64T, F134G, S177A, T180R
F64T, F134G, W153L, S177T, T180R
F64T, I79A, W153L, S175V, T180R
F64T, I79A, W153L, S177A, T180R
F64T, F134G, S175T, S177T, T180R
F64T, I79C, F134G, S177A
F64T, I79C, F134G, S177A, T180R
F64T, F134G, W153L, S177A, T180R, E284K
F64T, I79C, W153L, S175T, S177T, T180R
F64T, I79C, W153L, S175T, T180R
F64T, W153L, S175V, S177A
F64T, W153L, S175V, S177A, T180R
F64T, W153L, S175V, S177T
F64T, W153L, S175V, V188A, R190S
I79A, S177T
I79C, F134G
I79C, W153L
I79A, F134G, S175V
I79A, F134G, W153L
I79A, S175V, S177T
I79A, S177A, T180R
I79A, W153L, E284K
I79C, S175V, S177T
I79C, S177A, T180R
I79C, W153L, S175T
I79C, W153L, S177T
I79A, S175V, T180R
I79A, W153L, S175T, T180R
I79C, F134G, W153L, S175V
I79C, S175T, S177T, T180R
I79C, F134G, S177A, T180R
I79C, W153L, S177T, T180R
I79A, W153L, S175V, S177T, T180R
I79C, F134G, S175T, S177A, T180R
I79C, F134G, W153L, S175T, T180I
I79C, W153L, S175V, S177T, T180R
I79N, F134G, S175V, S177T, T180R
I79A, F134G, W153L, S175T, S177T, T180R
F134G, S177T
F134G, T180R
F134G, W153L
F134G, W153L, S175V
F134G, W153L, S177T
F134G, S177T, E284R
F134G, S175T, T180R
F134G, W153L, S175T, S177A
F134G, W153L, S175T, S177T
F134G, W153L, S175T, T180R
F134G, W153L, S177T, T180R
F134G, S175T, S177T, T180R
F134G, S175V, S177A, T180R
F134G, W153L, S175V, S177T, T180R
W153L, S175T
W153L, T180R
W153L, S175V
W153L, S175T, S177T
W153L, S177A, T180R
S175V, S177T, T180R
W153L, S175T, S177A, T180R
W153L, S175V, S177A, T180R
W153L, S175V, S177T, T180R
S175V, S177T, T180R, E284K
S177A, T180R
S177T, T180R
W61A, F64G, I79A, F158A, S175G, S177A, T180L, N235V, E284D, A293K
W61A, F64G, I79A, F158A, S175T, S177G, T180V, N235V, E284K, A293G
W61A, F64G, I79A, F158G, S175A, S177A, T180L, N235C, E284D, A293V
W61A, F64G, I79A, F158G, S175A, S177G, T180L, N235C, E284K, A293G
W61A, F64G, I79A, F158G, S175G, S177T, T180R, N235K, E284R, A293G
W61A, F64G, I79C, F158A, S175A, S177A, T180V, N235K, E284R, A293V
W61A, F64G, I79C, F158A, S175A, S177T, T180R, N235C, E284D, A293G
W61A, F64G, I79C, F158G, S175A, S177T, T180V, N235K, E284D, A293G
W61A, F64G, I79C, F158G, S175T, S177T, T180R, N235C, E284K, A293G
W61A, F64L, I79A, F158A, S175G, S177T, T180V, N235C, E284R, A293G
W61A, F64L, I79A, F158G, S175A, S177T, T180V, N235K, E284D, A293K
W61A, F64L, I79A, F158G, S175G, S177A, T180L, N235C, E284D, A293G
W61A, F64L, I79A, F158G, S175T, S177G, T180V, N235C, E284K, A293V
W61A, F64L, I79C, F158A, S175A, S177A, T180L, N235C, E284D, A293V
W61A, F64L, I79C, F158A, S175A, S177G, T180V, N235C, E284D, A293G
W61A, F64L, I79C, F158A, S175G, S177A, T180R, N235C, E284R, A293G
W61A, F64L, I79C, F158A, S175G, S177A, T180V, N235C, E284D, A293G
W61A, F64L, I79C, F158A, S175V, S177A, T180R, N235C, E284D, A293G
W61A, F64L, I79C, F158G, S175A, S177A, T180L, N235K, E284R, A293G
W61A, F64L, I79C, F158G, S175G, S177A, T180R, N235K, E284D, A293G
W61A, F64L, I79C, F158G, S175V, S177A, T180V, N235C, E284K, A293K
W61A, F64M, I79A, F158A, S175A, S177A, T180L, N235C, E284D, A293G
W61A, F64M, I79A, F158A, S175A, S177G, T180L, N235C, E284D, A293V
W61A, F64M, I79A, F158A, S175A, S177G, T180V, N235V, E284R, A293G
W61A, F64M, I79A, F158A, S175A, S177T, T180V, N235C, E284D, A293K
W61A, F64M, I79A, F158A, S175G, S177G, T180L, N235C, E284D, A293V
W61A, F64M, I79A, F158A, S175V, S177T, T180R, N235C, E284R, A293K
W61A, F64M, I79A, F158A, S175V, S177T, T180R, N235V, E284D, A293G
W61A, F64M, I79A, F158G, S175A, S177G, T180R, N235K, E284R, A293G
W61A, F64M, I79A, F158G, S175G, S177T, T180L, N235K, E284R, A293G
W61A, F64M, I79C, F158A, S175G, S177T, T180R, N235V, E284R, A293G
W61A, F64M, I79C, F158A, S175V, S177T, T180R, N235C, E284K, A293G
W61A, F64M, I79C, F158G, S175A, S177A, T180L, N235C, E284R, A293G
W61A, F64M, I79C, F158G, S175A, S177A, T180R
W61A, F64M, I79C, F158G, S175A, S177T, T180L, N235C, E284R, A293G
W61A, F64M, I79C, F158G, S175V, S177A, T180V, N235V, E284D, A293G
W61A, F64T, I79A, F158A, S175A, S177T, T180R, N235K, E284D, A293G
W61A, F64T, I79A, F158A, S175G, S177T, T180L, N235C, E284D, A293G
W61A, F64T, I79A, F158A, S175V, S177G, T180R, N235C, E284D, A293G
W61A, F64T, I79A, F158G, S175A, S177A, T180L, N235C, E284K, A293V
W61A, F64T, I79C, F158A, S175G, S177A, T180L, N235V, E284D, A293G
W61A, F64T, I79C, F158A, S175T, S177T, T180L, N235C, E284D, A293G
W61A, F64T, I79C, F158A, S175V, S177A, T180L, N235V, E284R, A293G
W61A, F64T, I79C, F158G, S175A, S177T, T180R, N235K, E284K, A293V
W61A, F64T, I79C, F158G, S175G, S177A, T180L, N235C, E284K, A293G
W61A, F64T, I79C, F158G, S175T, S177T, T180R, N235K, E284D, A293G
W61A, F64T, I79C, F158G, S175V, S177T, T180V, N235K, E284D, A293K
W61A, F64W, I79A, F158A, S175T, S177G, T180V, N235V, E284D, A293V
W61A, F64W, I79A, F158G, S175A, S177A, T180L, N235K, E284D, A293V
W61A, F64W, I79A, F158G, S175V, S177T, T180R, N235C, E284R, A293G
W61A, F64W, I79C, , F158G, S175G, S177G, T180L, N235K, E284D, A293V
W61A, F64W, I79C, F158A, S175A, S177A, T180R, N235C, E284D, A293G
W61A, F64W, I79C, F158A, S175V, S177A, T180L, N235K, E284D, A293G
W61A, F64W, I79C, F158G, S175A, S177A, T180L, N235K, E284D, A293G
W61A, F64W, I79C, F158G, S175G, S177T, T180R, N235C, E284R, A293G
W61V, F64G, I79A, F158G, S175V, S177A, T180R, N235V, E284D, A293G
W61V, F64G, I79C, F158A, S175T, S177G, T180V, N235K, E284D, A293G
W61V, F64L, I79A, F158A, S175A, S177G, T180R, N235C, E284R, A293G
W61V, F64L, I79A, F158A, S175G, S177G, T180L, N235K, E284R, A293K
W61V, F64L, I79A, F158A, S175G, S177G, T180V, N235K, E284D, A293G
W61V, F64L, I79A, F158G, S175A, S177G, T180R, N235C, E284R, A293G
W61V, F64L, I79A, F158G, S175A, S177T, T180L, N235C, E284D, A293G
W61V, F64L, I79A, F158G, S175G, S177G, T180L, N235V, E284D, A293G
W61V, F64M, I79A, F158A, S175A, S177G, T180R, N235C, E284D, A293G
W61V, F64M, I79A, F158A, S175G, S177A, T180V, N235V, E284R, A293G
W61V, F64M, I79A, F158A, S175T, S177A, T180L, N235C, E284K, A293V
W61V, F64M, I79A, F158G, S175A, S177G, T180V, N235C, E284R, A293V
W61V, F64M, I79A, F158G, S175G, S177G, T180V, N235K, E284K, A293G
W61V, F64M, I79C, F158A, S175A, S177G, T180R, N235K, E284D, A293G
W61V, F64M, I79C, F158A, S175G, S177T, T180L, N235V, E284R, A293G
W61V, F64M, I79C, F158G, S175V, S177T, T180R, N235C, E284K, A293V
W61V, F64T, I79A, F158G, S175A, S177T, T180L, N235C, E284D, A293G
W61V, F64T, I79A, F158G, S175T, S177A, T180V, N235K, E284D, A293G
W61V, F64T, I79C, F158A, S175T, S177A, T180L, N235C, E284D, A293G
W61V, F64T, I79C, F158A, S175T, S177A, T180V, N235C, E284D, A293G
W61V, F64T, I79C, F158G, S175A, S177A, T180L, N235V, E284K, A293G
W61V, F64T, I79C, F158G, S175G, S177T, T180R, N235C, E284R, A293G
W61V, F64W, I79A, F158G, S175V, S177T, T180R, N235C, E284D, A293K
W61V, F64W, I79C, F158G, S175A, S177T, T180V, N235C, E284K, A293G
W61V, F64W, I79C, F158G, S175A, S177T, T180V, N235V, E284K, A293K
W61V, F64W, I79C, F158A, S175T, S177T, T180R, N235K, E284R, A293G
F158A, S175A, S177G, T180V, N235K, E284R, A293G
F158A, S175A, S177T, T180L, N235C, E284D, A293V

In at least one embodiment, the recombinant polypeptides having prenyltransferase activity, increased activity, and one or more residue differences as compared to the reference prenyltransferase polypeptide of SEQ ID NO: 20 at one or more positions selected from W61, F64, I79, F134, W153, F158, S175, S177, T180, N235, E284, and A293 can further comprise an amino acid residue difference as compared to SEQ ID NO: 20 at one or more positions selected from: P5, H7, D10, N11, K34, C41, R46, F49, N50, R52, L54, G58, F65, V68, F75, M80, D87, I91, K93, D95, V99, I105, E106, I113, V115, I121, T123, K125, A129, F138, I140, F144, F161, I165, F173, Y176, S181, V188, R190, F193, S194, F195, I196, I197, M200, G204, M205, S214, E217, D219, T229, F238, S241, V243, L249, S251, S253, W258, S264, M267, F276, C277, L278, F280, Q281, T282, A286, L287, A288, Y290, A291, P294, S295, F299, F301, I302, W303, L304, L305, Y307, A308, E309, Y310, F311, V312, Y313, V314, and F315. In at least one embodiment, the further amino acid differences can be selected from: P5G, P5V, H7C, D10L, D10V, D10W, N11D, K34E, C41A, C41G, C41S, R46K, F49L, F49M, F49R, N50D, R52P, L54S, G58S, F65L, V68D, F75W, M80V, D87E, I91V, K93N, D95N, V99A, I105V, E106R, I113N, I113W, V115A, I121T, T123K, K125M, K125V, K125W, A129T, F138I, I140T, F144S, F161V, I165L, I165T, F173I, Y176S, S181R, V188A, V188S, R190A, R190G, R190Q, R190S, F193L, S194A, S194L, S194V, F195V, I196T, I197T, M200R, G204A, G204S, M205G, M205R, S214C, E217G, D219V, T229V, F238L, F238W, S241F, V243A, L249A, L249V, S251A, S251C, S253P, W258R, S264Y, M267T, F276L, C277A, C277M, C277R, L278P, F280G, F280L, F280R, Q281R, T282P, A286G, L287F, A288P, Y290S, A291E, P294E, S295A, F299L, F301S, I302L, W303C, L304R, L305S, Y307H, Y307S, A308E, A308P, A308R, E309V, Y310C, Y310P, Y310S, F311P, F311S, V312G, Y313H, Y313P, V314A, and F315S.

Based on the correlation of recombinant polypeptide functional information provided herein with the sequence information provided in Table 3, the accompanying Sequence Listing, and/or the Examples disclosed herein, one of ordinary skill can recognize that the present disclosure provides a range of recombinant polypeptides having prenyltransferase activity, wherein the polypeptide comprises an amino acid sequence comprising one or more of the amino acid differences or sets of amino acid differences (relative to SEQ ID NO: 20) disclosed in any one of SEQ ID NO: 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114,116, 118, 120, 122, 124,126, 128, 130, 132, 134,136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, and 514, and otherwise have at least 80%, at least 85% at least 90%, at least 95%, at least 97%, at least 98%, or at least 99% identity to a sequence selected from the group consisting of SEQ ID NO: 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, and 514.

Thus, in at least one embodiment, a recombinant polypeptide of the present disclosure having prenyltransferase activity can have an amino acid sequence comprising one or more of the amino acid differences or sets of amino acid differences (relative to SEQ ID NO: 20) disclosed in any one of SEQ ID NO: 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, and 514, and additionally have 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, 1-50, 1-55, or 1-60 residue differences at other residue positions. In some embodiments, the number of differences can be 1, 2, 3, 4, 5, 6, 7, 8, 9, 10, 11, 12, 14, 15, 16, 18, 20, 22, 24, 26, 30, 35, 40, 45, 50, 55, or 60 residue differences at the other residue positions.

In addition to the residue positions specified above, any of the engineered prenyltransferase polypeptides disclosed herein can further comprise other residue differences relative to the reference polypeptide of SEQ ID NO:20 at other residue positions.

Residue differences at these other residue positions can provide for additional variations in the amino acid sequence without adversely affecting the ability of the recombinant polypeptide to carry out the desired biocatalytic conversion (e.g., conversion of compound (2) to compound (1)). In some embodiments, the recombinant polypeptides can have additionally 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40 residue differences at other amino acid residue positions as compared to SEQ ID NO: 10. In some embodiments, the number of differences can be 1, 2, 3, 4, 5, 6, 7, 8, 9, 10, 11, 12, 14, 15, 16, 18, 20, 22, 24, 26, 30, 35, and 40 residue differences at other residue positions. The residue difference at these other positions can include conservative changes or non-conservative changes. In some embodiments, the residue differences can comprise conservative substitutions and non-conservative substitutions as compared to the reference polypeptide of SEQ ID NO: 20.

In some embodiments, the recombinant polypeptides of the disclosure can be in the form of fusion polypeptides in which the engineered polypeptides are fused to other polypeptides, such as, by way of example and not limitation, antibody tags (e.g., myc epitope), purification sequences (e.g., His tags for binding to metals), and cell localization signals (e.g., secretion signals). Thus, the recombinant polypeptides described herein can be used with or without fusions to other polypeptides. It is also contemplated that the recombinant polypeptides described herein are not restricted to the genetically encoded amino acids. In addition to the genetically encoded amino acids, the polypeptides described herein may be comprised, either in whole or in part, of naturally-occurring and/or synthetic non-encoded amino acids.

In at least one embodiment, it is contemplated that the recombinant polypeptides having prenyltransferase activity of the present disclosure can be expressed as a fusion with a polypeptide having farnesyl pyrophosphate synthetase (FPP synthase) activity, such as the Erg20 polypeptide of Saccharomyces cerevisiae, or a variant thereof, such the well-known variant, Erg20ww of SEQ ID NO: 526. As disclosed elsewhere herein, including the Examples, a nucleic acid encoding an N-terminal fusion of Erg20ww and a recombinant polypeptide having prenyltransferase activity of the present disclosure can be genomically integrated in a yeast strain to provide a pathway for the synthesis of CBGA and other cannabinoids.

In another aspect, the present disclosure provides polynucleotides encoding the recombinant polypeptides having prenyltransferase activity and increased activity and/or yield as described herein. In at least one embodiment, the polynucleotide encoding a recombinant polypeptide having prenyltransferase activity comprises an amino acid sequence that is at least about 80%, 85%, 90%, 91%, 92%, 93%, 94%, 95%, 96%, 97%, 98%, 99%, or more identical to the polypeptide sequence of SEQ ID NO:20. In some embodiments, the polynucleotide encodes a recombinant polypeptide comprising an amino acid sequence that has the percent identity described above and has one or more amino acid residue differences as compared to SEQ ID NO:20 described elsewhere herein.

In at least one embodiment, the polynucleotide has a sequence encoding a recombinant polypeptide that does not include an amino acid difference relative to SEQ ID NO: 20, but which polynucleotide sequence has one or more codon differences relative to SEQ ID NO: 19, which codon differences result in increased yield of the prenylated cannabinoid product produced by a recombinant host cell in which the polynucleotide sequence is integrated. In at least one embodiment, the polynucleotide has a sequence of at least 80% identity to SEQ ID NO: 19, and a codon difference as compared to SEQ ID NO: 19 at a position encoding an amino acid residue selected from: V33, I37, F73, N74, A78, Q82, K93, P97, V99, S104, L111, L117, G119, F132, V133, I137, G139, F141, R152, Q155, N160, S166, A182, T201, G218, I213, V224, S225, A233, G242, V261, K263, F276, S295, L304, Y306, F311, and V312. In at least one embodiment, the codon differences at positions V33, I37, F73, N74, A78, Q82, K93, P97, V99, S104, L111, L117, G119, F132, V133, I137, G139, F141, R152, Q155, N160, S166, A182, T201, G218, I213, V224, S225, A233, G242, V261, K263, F276, S295, L304, Y306, F311, and V312 are selected from: V33 (GTT>GTC), I37 (ATT>ATC), F73 (TTT>TTC), N74 (AAT>AAC), A78 (GCA>GCG), Q82 (CAA>CAG), K93 (AAG>AAA), P97 (CCA>CCG), V99 (GTT>GTC), S104 (TCA>TCT), L111 (TTA>TTG), L117 (TTG>CTG), G119 (GGT>GGC), F132F (TTC>TTT), V133 (GTT>GTC), G139 (GGT>GGG), R152 (AGA>CGT), Q155 (CAA>CAG), N160 (AAT>AAC), L162 (TTG>CTG), S166 (TCT>TCC), A182 (GCA>GCC), T201 (ACT>ACG), I213 (ATC>ATT), G218 (GGT>GGG), V224 (GTT>GTC), S225 (TCA>TCG), A233 (GCA>GCG), G242 (GGT>GGC), V261 (GTT>GTC), K263 (AAA>AAG), F276 (TTC>TTT), S295 (TCA>TCT), L304 (TTG>CTG), Y306 (TAT>TAC), F311 (TTT>TTC), and V312 (GTT>GTC).

It is also contemplated that the polynucleotides encoding the recombinant polypeptides having prenyltransferase activity and increased activity and/or yield as described herein, can include a combination of one or more codon differences relative to SEQ ID NO: 19, wherein at least one the codon differences encodes an amino acid difference as compared to SEQ ID NO: 20 and at least one codon difference does not encode an amino acid difference as compared to SEQ ID NO: 20 Accordingly, in at least one embodiment, the present disclosure provides a polynucleotide sequence encoding a recombinant polypeptide having prenyltransferase activity, wherein the polynucleotide sequence comprises a combination of a codon difference encoding an amino acid difference and a codon difference selected from: G58S and F73 (TTT>TTC); and G139 (GGT>GGG) and S175V.

In at least one embodiment, the polynucleotide comprises a sequence encoding an exemplary recombinant polypeptide having prenyltransferase activity as disclosed in Table 3 and accompanying Sequence Listing. In at least one embodiment, the polynucleotide comprises a sequence of at least 80%, at least 85%, at least 90%, at least 95%, at least 97%, at least 98%, or at least 99% identity to a sequence selected from the group consisting of SEQ ID NO: 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121,123, 125, 127, 129, 131,133, 135, 137, 139, 141,143, 145, 147, 149, 151, 153, 155, 157, 159, 161, 163, 165, 167, 169, 171, 173, 175, 177, 179, 181, 183, 185, 187, 189, 191, 193, 195, 197, 199, 201,203, 205, 207, 209, 211,213, 215, 217, 219, 221,223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, and 513. In at least one embodiment, the polynucleotide comprises a codon degenerate sequence of a sequence selected from the group consisting of SEQ ID NO: 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121,123, 125, 127, 129, 131,133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, and 513.

The polynucleotide sequences encoding the recombinant polypeptides of the present disclosure may be operatively linked to one or more heterologous regulatory sequences that control gene expression to create a recombinant polynucleotide capable of expressing the polypeptide. Expression constructs containing a heterologous polynucleotide encoding the recombinant polypeptide can be introduced into appropriate host cells to express the corresponding polypeptide. Because of the knowledge of the codons corresponding to the various amino acids, availability of a protein sequence provides a description of all the polynucleotides capable of encoding the subject. The degeneracy of the genetic code, where the same amino acids are encoded by alternative or synonymous codons allows an extremely large number of nucleic acids to be made, all of which encode the improved transaminase enzymes disclosed herein. Thus, having identified a particular amino acid sequence, those skilled in the art could make any number of different nucleic acids by simply modifying the sequence of one or more codons in a way which does not change the amino acid sequence of the protein. In this regard, the present disclosure specifically contemplates each and every possible variation of polynucleotides that could be made by selecting combinations based on the possible codon choices, and all such variations are to be considered specifically disclosed for any polypeptide disclosed herein, including the amino acid sequences presented in Table 3 and the accompanying Sequence Listing.

The codons can be selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. It is contemplated that all codons need not be replaced to optimize the codon usage of the recombinant polypeptide since the natural sequence will comprise preferred codons and because use of preferred codons may not be required for all amino acid residues. Consequently, codon optimized polynucleotides encoding the recombinant polypeptide may contain preferred codons at about 40%, 50%, 60%, 70%, 80%, or greater than 90% of codon positions of the full length coding region.

The present disclosure also provides an expression vector comprising a polynucleotide encoding a recombinant polypeptide having prenyltransferase activity and increased thermostability, and one or more expression regulating regions such as a promoter, a terminator, a replication origin, or the like, depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the recombinant polypeptide at such sites. Alternatively, a polynucleotide sequence of the present disclosure may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression. The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one which, when introduced into the host cell, is integrated into the genome, and replicated together with the chromosome(s) into which it has been integrated. Furthermore, a single vector or plasmid or two or more vectors or plasmids which together contain the total DNA to be introduced into the genome of the host cell, or a transposon may be used. In at least one embodiment, the expression vector further comprises one or more selectable markers, which permit easy selection of transformed cells.

The present disclosure also provides host cell comprising a polynucleotide or expression vector encoding a recombinant polypeptide of the present disclosure, wherein the polynucleotide is operatively linked to one or more control sequences for expression of the polypeptide having prenyltransferase activity in the host cell. Host cells for use in expressing the polypeptides encoded by the expression vectors of the present invention are well known in the art and include but are not limited to, bacterial cells, such as E. coli, or fungal cells, such as Saccharomyces cerevisiae or Pichia pastoris, insect cells, such as Drosophila S2 and Spodoptera Sf9, animal cells, such as CHO, COS, BHK, 293, and plant cells. Appropriate culture mediums and growth conditions for the above-described host cells are well known in the art. Accordingly, in at least one embodiment, the present disclosure provides a method for producing a cannabinoid comprising: (a) culturing in a suitable medium a recombinant host cell of the present disclosure; and (b) recovering the produced cannabinoid.

Use in Recombinant Host Cells

The recombinant polynucleotides of the present disclosure that encode recombinant polypeptides having prenyltransferase activity can be incorporated into recombinant host cells for enhanced in vivo cannabinoid biosynthesis. In the context of recombinant host cells, the recombinant polynucleotides can be incorporated into a pathway capable of producing a cannabinoid precursor, and thereby provide the prenyltransferase activity for biosynthesis of cannabinoids by the cells. As described elsewhere herein, the recombinant polypeptides encoded by the recombinant polynucleotides having prenyltransferase activity of the present disclosure when integrated into recombinant host cells with a pathway that converts hexanoic acid (HA) to the cannabinoid precursor, olivetolic acid (OA) exhibit enhanced yields of prenylated cannabinoid product, CBGA.

Generally, the cannabinoid pathway of the recombinant host cell is made up of a sequence of linked enzymes that produce a cannabinoid precursor substrate (e.g., OA) and then convert that precursor to a prenylated cannabinoid compound (e.g., CBGA). Accordingly, the pathway comprises at least a prenyltransferase capable of prenylating the aromatic cannabinoid precursor using a prenyl donor substrate, such as GPP. Further enzymatic modification of the initial prenylated cannabinoid compound by cannabinoid synthases (e.g., CBDAS) can also be part of the cannabinoid pathway. As described elsewhere herein, it is contemplated that a wide range of cannabinoid compounds can be produced biosynthetically by a recombinant host cell integrated with such a cannabinoid pathway. Methods and techniques for integrated polynucleotides expressing pathway enzymes into recombinant host cells, such as yeast, are well known in the art and described elsewhere herein including the Examples.

In at least one embodiment, the pathway integrated in the host cell can comprise a nucleic acid encoding a farnesyl pyrophosphate synthetase (FPP synthase) polypeptide capable of producing the prenyltransferase substrate GPP. One well-known FPP synthase is Erg20 polypeptide from S. cerevisiae, or its well-known variant, Erg20ww (SEQ ID NO: 526). As disclosed elsewhere herein, including the Examples, in at least one embodiment of the recombinant host cells of the present disclosure, a nucleic acid encoding a FPP synthase can be integrated into the host cell as an N-terminal fusion with the recombinant polypeptide having prenyltransferase activity. For example, the present disclosure exemplifies yeast strains integrated with a CBGA producing pathway that includes a nucleic acid encoding an N-terminal fusion of Erg20ww (SEQ ID NO: 526) with the recombinant variant prenyltransferase polypeptides of Table 3 of the present disclosure.

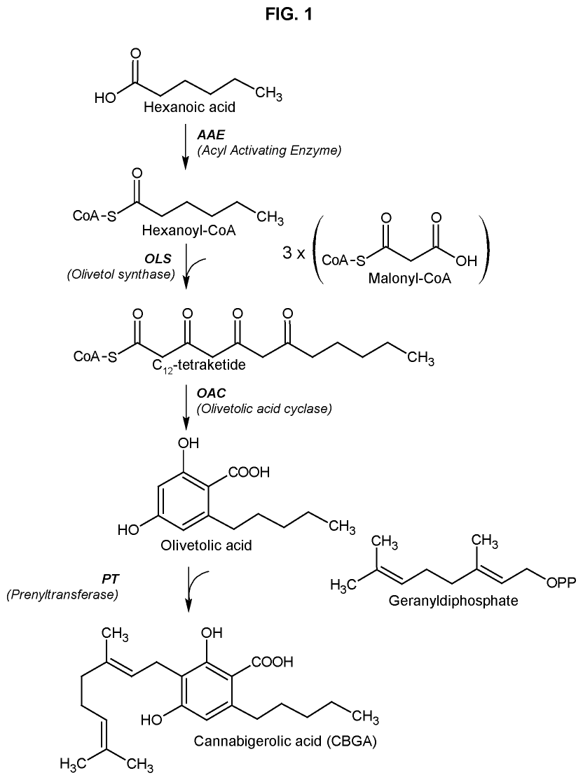

One exemplary cannabinoid pathway is depicted in FIG. 1. As shown in FIG. 1, this pathway is capable of converting hexanoic acid (HA) to the cannabinoid, cannabigerolic acid (CBGA). The pathway of FIG. 1 includes the sequence of three enzymes: (1) acyl activating enzyme (AAE), a CoA ligase enzyme of class E.C. 6.2.1.1; (2) olivetol synthase (OLS), a CoA synthase enzyme of class E.C. 2.3.1.206; and (3) olivetolic acid cyclase (OAC), a carbon-sulfur lyase enzyme of class E.C. 4.4.1.26. These three enzymes carry out the conversion of the HA starting compound to the cannabinoid precursor compound, OA. When prenyltransferase (PT), a transferase of class E.C. 2.5.1.102, is added to this three enzyme pathway, its activity can catalyze the prenylation of OA with geranyl pyrophosphate (GPP), thereby forming the cannabinoid compound, CBGA. It is contemplated that any of the recombinant polynucleotides of the present disclosure that encode recombinant polypeptides having prenyltransferase activity can be incorporated in such a four enzyme pathway to express the necessary prenyltransferase activity for cannabinoid biosynthesis.

Accordingly, in at least one embodiment, the present disclosure provides a recombinant host cell comprising recombinant polynucleotides encoding a pathway capable of producing a cannabinoid, wherein the pathway comprises enzymes capable of catalyzing reactions (i)-(iv):

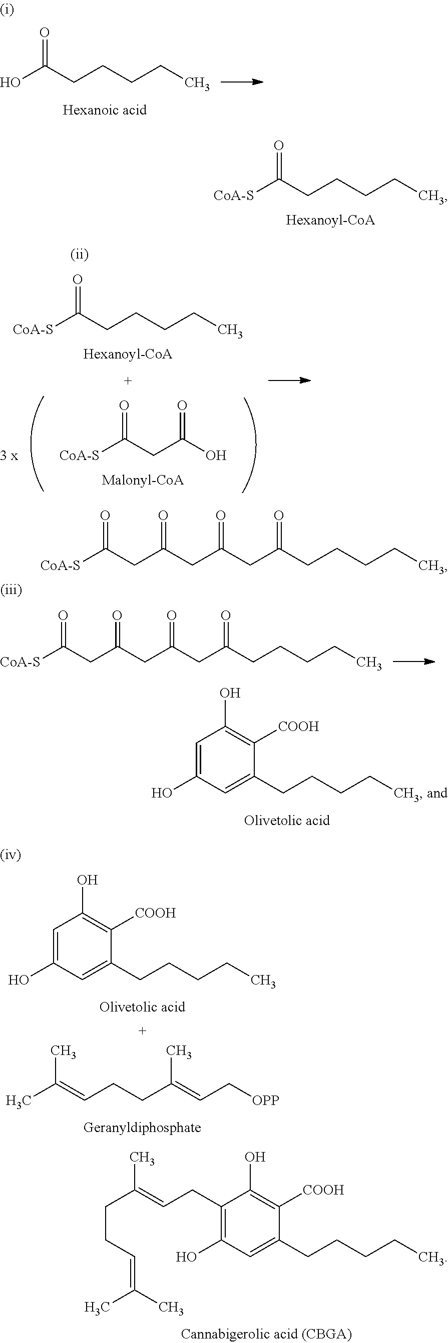

As shown in FIG. 1, exemplary enzymes capable of catalyzing reactions (i)-(iv) are: (i) acyl activating enzyme (AAE); (ii) olivetol synthase (OLS); (iii) olivetolic acid cyclase (OLA); and (iv) prenyltransferase (PT). In at least one embodiment, the prenyltransferase of the pathway of the recombinant host cell is a recombinant polypeptide having prenyltransferase activity of the present disclosure, such as an exemplary recombinant polypeptide as disclosed in Table 3.

In at least one embodiment, it is contemplated that a recombinant host cell comprising a pathway of only the three enzymes, AAE, OLS, and OAC, could modified by integrating a recombinant polynucleotide of the present disclosure to provide expression of a recombinant polypeptide with the prenyltransferase activity to convert OA to CBGA, thereby providing a four enzyme cannabinoid pathway as depicted in FIG. 1.

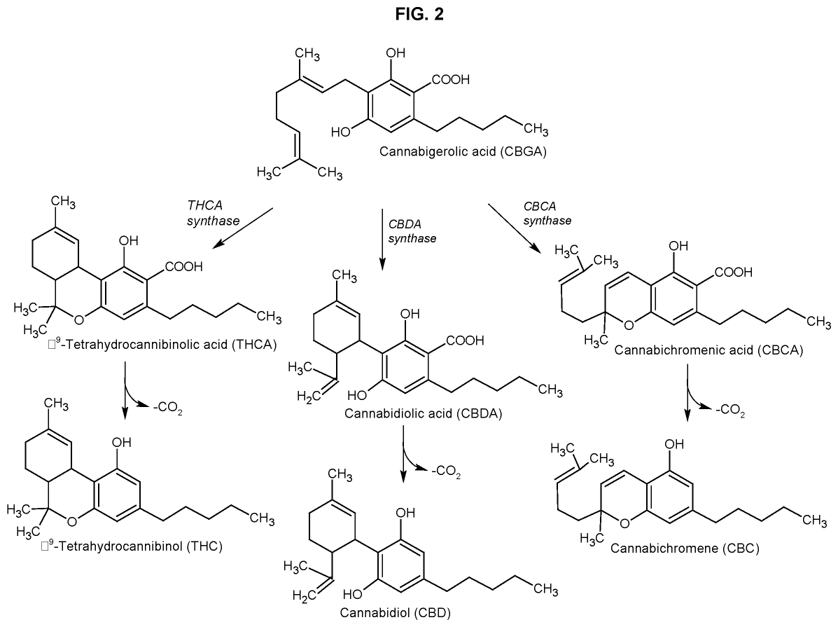

As shown in FIG. 2, the cannabinoid compound, CBGA, that is produced by the pathway of FIG. 1, can be further converted to at least three other different cannabinoid compounds, Δ⁹-tetrahydrocannabinolic acid (THCA), cannabidiolic acid (CBDA), and/or cannabichromenic acid (CBCA). Accordingly, in at least one embodiment, the present disclosure provides a recombinant host cell comprising a pathway capable of converting hexanoic acid to CBGA and further comprising an enzyme capable of catalyzing the conversion of (v) CBGA to Δ⁹-THCA; (vi) CBGA to CBDA; and/or (vii) CBGA to CBCA. Thus, in at least one embodiment, the recombinant host cell comprises pathway capable of converting hexanoic acid to CBGA further comprises further comprises enzymes capable of catalyzing a reaction (v), (vi), and/or (vii):

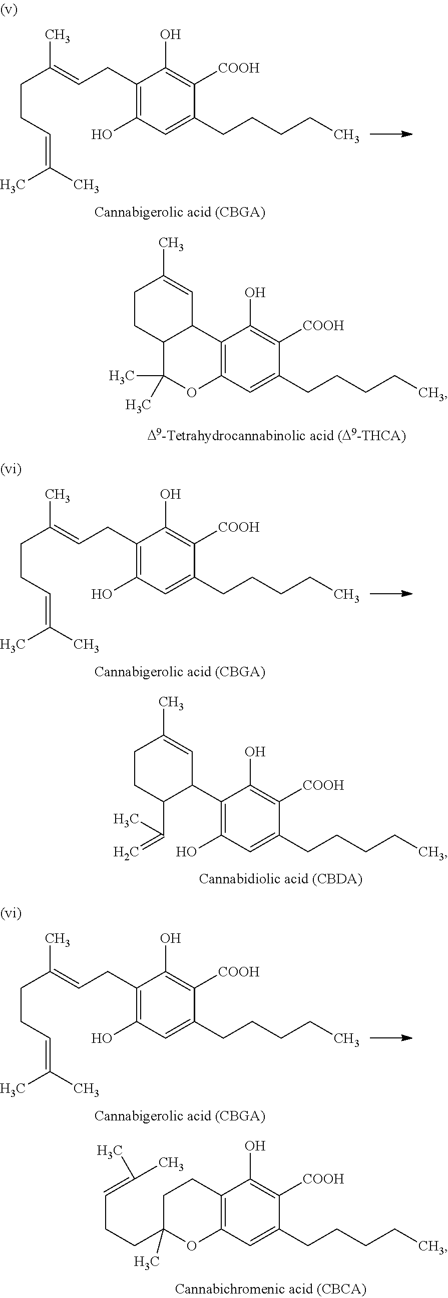

As shown in FIG. 2, exemplary enzymes capable of catalyzing reaction (v)-(vii) are: (v) THCA synthase (THCAS); (vi) CBDA synthase (CBDAS); and (vii) CBCA synthase (CBCAS). The extension of the four enzyme exemplary pathway of FIG. 1 with polynucleotide sequence capable of expressing such a cannabinoid synthase (e.g., CBDAS, THCAS, and/or CBCAS) allows for the biosynthetic production of one or more of the cannabinoids, Δ⁹-THCA, CBDA, and/or CBCA. These cannabinoids can then be decarboxylated to provide the cannabinoids, Δ⁹-THC, CBD, and/or CBC. Accordingly, it is contemplated, that in some embodiments this further decarboxylation reaction can be carried out under in vitro reaction conditions using the cannabinoid acids separated and/or isolated from the recombinant host cells.

Exemplary cannabinoid pathway enzymes that can be introduced into a recombinant host cell to provide the pathways illustrated in FIGS. 1 and 2 include, but are not limited to, the enzymes derived from C. sativa, AAE1, OLS, OAC, PT4, CBDAS, and/or THCAS, listed in Table 4 (below), and homologs and variants of these enzymes, as described elsewhere herein.

TABLE 4
Exemplary cannabinoid pathway enzymes

| | | SEQ | SEQ |
|---|---|---|---|
| | | ID | ID |
| Name | Source | NO: | NO: |
| (type) | (accession) | (nt) | (aa) |
| AAE1 | Cannabis sativa | 1 | 2 |
| (acyl activating enzyme) | (AFD33345.1) | | |
| OLS | Cannabis sativa | 3 | 4 |
| (olivetol synthase) | (BAG14339.1) | | |
| OAC | Cannabis sativa | 5 | 6 |
| (olivetolic acid cyclase) | AFN42527.1) | | |
| PT4 | Cannabis sativa | 7 | 8 |
| (aromatic | (DAC76710.1) | | |
| prenyltransferase) | | | |
| d82_PT4 | 82 aa N-term truncation | 9 | 10 |
| (aromatic | of SEQ ID NO: 8 | | |
| prenyltransferase) | | | |
| CBDAS | Cannabis sativa | 11 | 12 |
| (CBDA synthase) | (BAF65033.1) | | |
| d28_CBDAS | 28 aa N-term truncation of | 13 | 14 |
| (CBDA synthase) | SEQ ID NO: 12 | | |
| THCAS | Cannabis sativa | 15 | 16 |
| (THCA synthase) | (BAC41356.1) | | |
| d28_THCAS | 28 aa N-term truncation of | 17 | 18 |
| (THCA synthase) | SEQ ID NO: 16 | | |

The sequences of the exemplary cannabinoid pathway enzymes AAE1, OLS, OAC, PT4, CBDAS, and THCAS listed in Table 4 are naturally occurring sequences derived from the plant source, Cannabis sativa. In the recombinant host cell embodiments of the present disclosure, it is contemplated that the PT4 enzyme of SEQ ID NO: 10 is replaced in the host cell by a recombinant polynucleotide encoding a recombinant polypeptide having prenyltransferase activity of the present disclosure. It is contemplated that the other heterologous cannabinoid pathway enzymes used in the recombinant host can include enzymes derived from naturally occurring sequence homologs of the AAE1, OLS, OAC, CBDAS, THCAS, CBCAS. For example, based on the sequence, accession, and enzyme classification information provided herein, one of ordinary skill can identify known naturally occurring homologs to AAE1, OLS, OAC, CBDAS, THCAS, CBCAS having activity in the desired biocatalytic reaction. Further, it is contemplated that the pathway enzymes AAE1, OLS, OAC, CBDAS, THCAS, CBCAS, or their homologs, as used in the recombinant host can include enzymes having non-naturally occurring sequences. For example, enzymes with amino acid sequences engineered to function optimally in a particular enzyme pathway, and/or optimally for production of particular cannabinoid, and/or optimally in a particular host. Methods for preparing such non-naturally occurring enzyme sequences are known in the art and include methods for enzyme engineering such as directed evolution (see, e.g., Stemmer, 1994, Proc Natl Acad Sci USA 91:10747-10751; PCT Publ. Nos. WO 95/22625, WO 97/0078, WO 97/35966, WO 98/27230, WO 00/42651, and WO 01/75767; U.S. Pat. Nos. 6,537,746; 6,117,679; 6,376,246; and 6,586,182; and U.S. Pat. Publ. Nos. 20080220990A1 and 20090312196A1; each of which is hereby incorporated by reference herein). Other modifications of cannabinoid pathway enzymes contemplated by the present disclosure include modification of the enzyme's amino acid sequence at either its N- or C-terminus by truncation or fusion. For example, in at least one embodiment of the pathway of producing a cannabinoid, versions of the AAE1, OLS, OAC, and/or CBDAS enzymes that are engineered with amino acid substitutions and/or truncated at the N- or C-terminus can be prepared using methods known in the art, and used in the compositions and methods of the present disclosure. In one embodiment, a CBDAS enzyme of SEQ ID NO: 12 that is truncated at the N-terminus by 28 amino acids to delete the native signal peptide can be used. The amino acid sequence of such a truncated CBDAS is provided herein as the d28_CBDAS enzyme of SEQ ID NO: 14. Accordingly, in at least one embodiment of the recombinant host cell, the pathway capable of producing a cannabinoid comprises at least enzymes having an amino acid sequence at least 90% identity to SEQ ID NO: 2 (AAE1), SEQ ID NO: 4 (OLS), SEQ ID NO: 6 (OAC), and an amino acid sequence of at least 90% identity to recombinant polypeptide of the present disclosure as provided in Table 3 and the accompanying Sequence Listing. Additionally, in at least one embodiment of the recombinant host cell, the pathway capable of producing a cannabinoid can further comprise a cannabinoid synthase of SEQ ID NO: 14 (d28_CBDAS) and/or SEQ ID NO: 18 (d28_THCAS).

Other cannabinoid pathway enzymes useful in the recombinant host cells and associated methods of the present disclosure are known in the art, and can include naturally occurring enzymes obtained or derived from cannabis plants, or non-naturally occurring enzymes that have been engineered based on the naturally occurring cannabis plant sequences. It is also contemplated that enzymes obtained or derived from other organisms (e.g., microorganisms) having a catalytic activity related to a desired conversion activity useful in a cannabinoid pathway can be engineered for use in a recombinant host cell of the present disclosure.

Although the cannabinoid pathways of FIGS. 1-2 depict the production of the more common naturally occurring cannabinoids, CBGA, Δ⁹-THCA, CBDA, and CBCA, it is also contemplated that the recombinant polypeptides, cannabinoid pathways, recombinant host cells, and associated methods of the present disclosure can also be used to biosynthesize a range of additional rarely occurring, and/or synthetic cannabinoid compounds. Table 1 depicts the names and structures of a wide range of exemplary rarely occurring, and/or synthetic cannabinoid compounds that are contemplated for production using the recombinant polypeptides, host cells, compositions, and methods of the present disclosure. Similarly, Table 2 depicts additional rarely occurring, and/or synthetic cannabinoid precursor compounds that could be produced by such recombinant host cells in the pathway for production of certain rarely occurring, and/or synthetic cannabinoid compounds of Table 1. Accordingly, in at least one embodiment, a recombinant host cell that includes a pathway to a cannabinoid precursor and that expresses a recombinant polypeptide having prenyltransferase activity of the present disclosure (e.g., as in Table 3) can be used for the biosynthetic production of a rarely occurring, and/or synthetic cannabinoid compound, or a composition comprising such a cannabinoid compound. It is contemplated that the produced rarely occurring, and/or synthetic cannabinoid compound can include, but is not limited to, the cannabinoid compounds of Table 1. Accordingly, in at least embodiment, a recombinant host cell of the present disclosure can be used for production of a cannabinoid compound selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁸-tetrahydrocannabinolic acid (Da-THCA), A-tetrahydrocannabinol (Da-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.

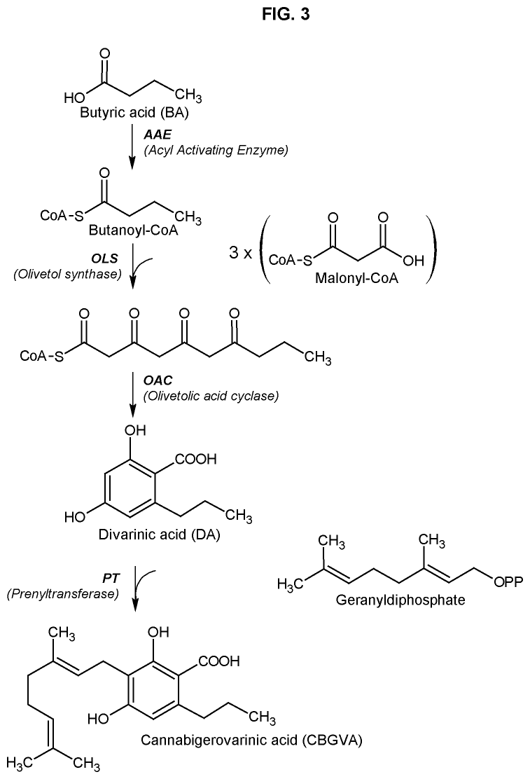

In at least one embodiment, the compositions and methods of the present disclosure can be used for the production of the rare varin series of cannabinoids, CBGVA, Δ⁹-THCVA, CBDVA, and CBCVA. As shown in Table 1, the varin cannabinoids feature a 3 carbon propyl side-chain rather than the 5 carbon pentyl side chain found in the common cannabinoids, CBGA, Δ⁹-THCA, CBDA, and CBCA. An exemplary cannabinoid pathway capable of producing the rare naturally occurring cannabinoid, cannabigerovarinic acid (CBGVA), is depicted in FIG. 3. Instead of starting with hexanoic acid, the pathway of FIG. 3 is fed butyric acid (BA) which is converted to divarinic acid (DA) via the same three enzyme pathway of AAE, OLS, and OAC. The cannabinoid precursor DA is then converted by an prenyltransferase to the rare cannabinoid, CBGVA. In at least one embodiment of the present disclosure, the prenyltransferase of the pathway of the recombinant host cell is a recombinant polypeptide having prenyltransferase activity of the present disclosure, such as an exemplary recombinant polypeptide as disclosed in Table 3. Accordingly, in at least one embodiment of the recombinant host cell, the pathway capable of producing a cannabinoid comprises enzymes capable of catalyzing reactions (i)-(iv):

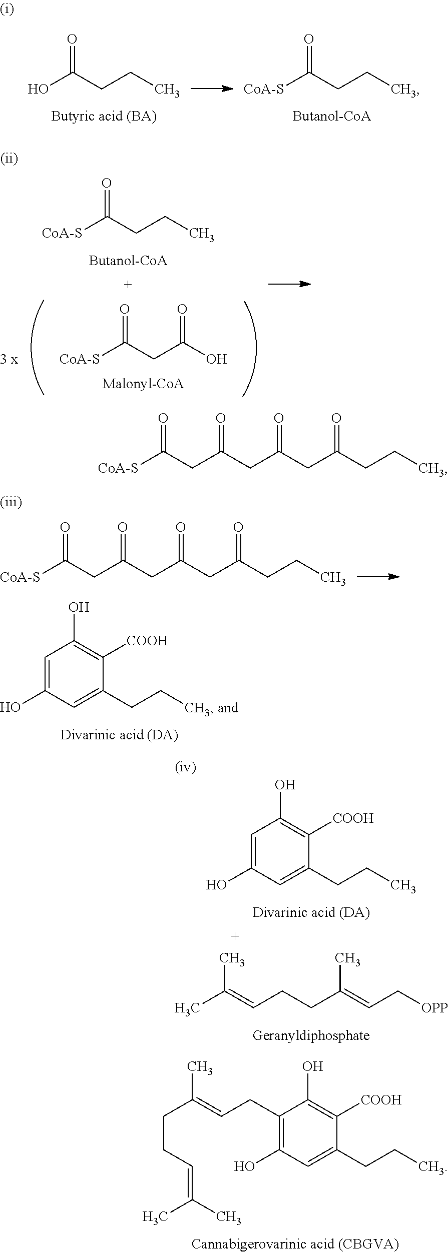

Exemplary enzymes capable of catalyzing reactions (i)-(iv) are: (i) acyl activating enzyme (AAE); (ii) olivetol synthase (OLS); (iii) olivetolic acid cyclase (OLA); and (iv) a recombinant polypeptide having prenyltransferase activity as disclosed herein (e.g., a polypeptide of Table 3). Exemplary enzymes, AAE, OLS, and OLA, derived from C. sativa are known in the art and also provided in Table 1 and the accompanying Sequence Listing.

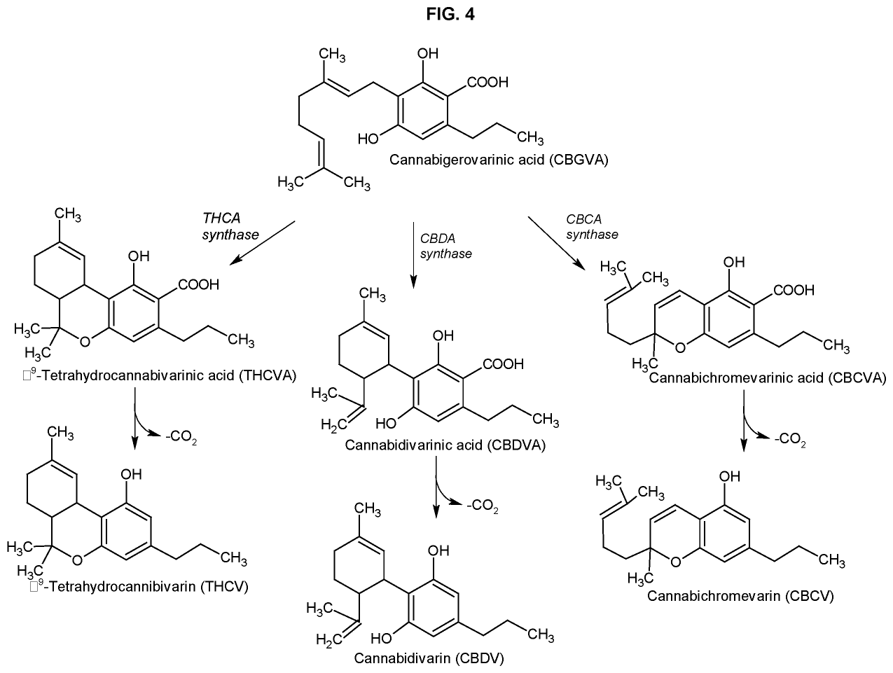

As further illustrated in FIG. 4, the heterologous pathway depicted in FIG. 3 which is capable of producing a rare cannabinoid, such as CBGVA, can be further modified to include one or more cannabinoid synthase enzymes (e.g., CBDAS, THCAS, CBCAS). As shown by the exemplary pathway of FIG. 4, with the incorporation of one or more synthase enzymes, the rare varin cannabinoid, CBGVA, can be converted to the rare varin cannabinoids, cannabidivarinic acid (CBDVA), Δ⁹-tetrahydrocannabivarinic acid (Δ⁹-THCVA), and cannabichromevarinic acid (CBCVA). Enzymes capable of carrying out these conversions include the C. sativa CBDA synthase, THCA synthase, and CBCA synthase, respectively. Accordingly, in at least one embodiment, the present disclosure provides a recombinant host cell comprising a pathway capable of converting BA to CBGVA and further comprising an enzyme capable of catalyzing the conversion of (v) CBGVA to Δ⁹-THCVA; (vi) CBGVA to CBDVA; and/or (vii) CBGVA to CBCVA. Thus, in at least one embodiment, the recombinant host cell comprises pathway capable of converting BA to CBGVA further comprises further comprises enzymes capable of catalyzing a reaction (v), (vi), and/or (vii):

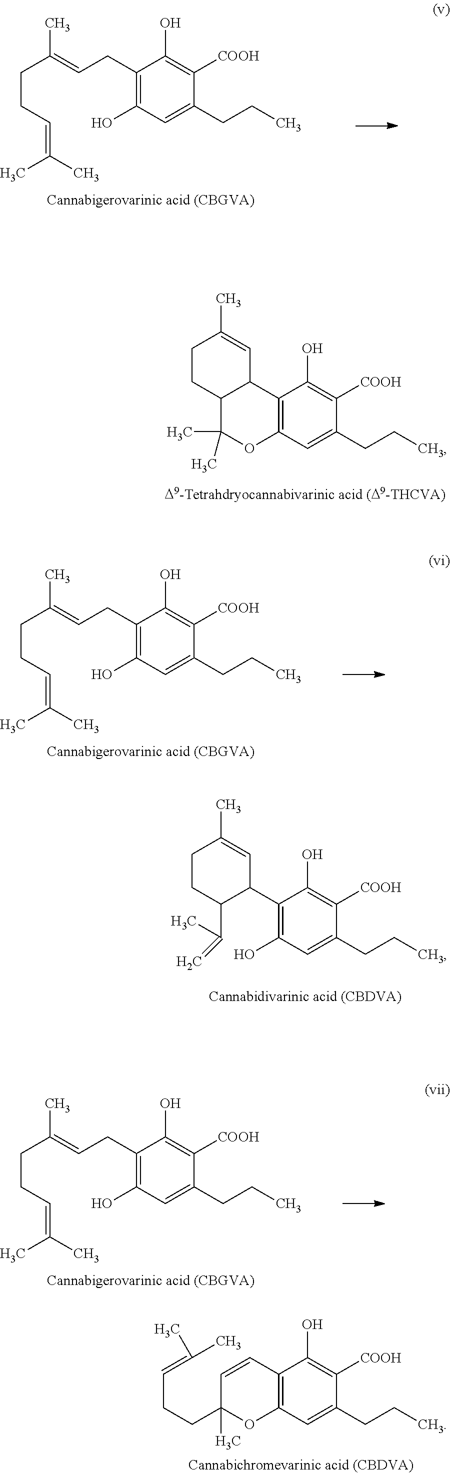

Exemplary enzymes capable of catalyzing reaction (v)-(vii) as shown above are: (v) THCA synthase (THCAS); (vi) CBDA synthase (CBDAS); and (vii) CBCA synthase (CBCAS). Exemplary THCAS, CBDAS, and CBCAS enzymes are provided in Table 1.

Furthermore, as shown in FIG. 4, the rare cannabinoid acids, CBDVA, Δ⁹-THCVA, and CBCVA, can undergo a further decarboxylation reaction to provide the varin cannabinoid products, cannabidivarin (CBDV), Δ⁹-tetrahydrocannabivarin (Δ⁹-THCV), and cannabichromevarin (CBCV), respectively. In some embodiments, this further decarboxylation can be carried out under in vitro reaction conditions using the cannabinoid acids isolated from the recombinant host cells.

Similarly, as shown in FIGS. 1 and 3, a heterologous cannabinoid pathway comprising the sequence of at least the four enzymes AAE, OLS, OAC, and PT (wherein, the PT is a recombinant polypeptide having prenyltransferase activity of the present disclosure) is capable of converting a precursor substrate compound, such as hexanoic acid (HA) to an initial cannabinoid compound, such as cannabigerolic acid (CBGA) or CBGVA. These initial cannabinoid product compounds can themselves be used as a substrate for the in vitro biosynthesis of a range of further cannabinoid product compounds, such as THCA and THCVA, as shown in FIGS. 2 and 4. A wide range of cannabinoid compounds, such as those shown in Table 1, are contemplated for in vivo biosynthetic production in a recombinant host cell of the present disclosure or via a partial or full in vitro biosynthesis process using recombinant polypeptides of the present disclosure.

As described herein, the heterologous cannabinoid pathways of the present disclosure can be incorporated (e.g., by recombinant transformation) into a range of host cells to provide a system for biosynthetic production of cannabinoids (e.g., CBGA, CBGVA, CBDA, CBDVA, THCA, THCVA). Generally, the host cell used in the recombinant host cells of the present disclosure can be any cell that can be recombinantly modified with nucleic acids and cultured to express the recombinant products of those nucleic acids, including polypeptides and metabolites produced by the activity of the recombinant polypeptides. A wide range of suitable sources of host cells are known in the art, and exemplary host cell sources useful as recombinant host cells of the present disclosure include, but are not limited to, Saccharomyces cerevisiae, Yarrowia lipolytica, Pichia pastoris, and Escherichia coli. It is also contemplated that the host cell source for a recombinant host cell of the present disclosure can include a non-naturally occurring cell source, e.g., an engineered host cell. For example, a non-naturally occurring source host cell, such as a yeast cell previously engineered for improved production of recombinant genes, may be used to prepare the recombinant host cell of the present disclosure.

The recombinant host cells of the present disclosure comprise heterologous nucleic acids encoding a pathway of enzymes capable of producing a cannabinoid precursor (e.g., OA or DA), and a heterologous nucleic acid comprising a sequence encoding a recombinant polypeptide having prenyltransferase activity capable of prenylating the cannabinoid precursor substrate using GPP as a co-substrate to form a cannabinoid product (e.g., CBGA or CBGVA). As described elsewhere herein, nucleic acid sequences encoding the cannabinoid pathway enzymes, are known in the art, and provided herein, and can readily be used in accordance with the present disclosure. Typically, the nucleic acid sequence encoding enzymes which form a part of a cannabinoid pathway, further include one or more additional nucleic acid sequences, for example, a nucleic acid sequence controlling expression of the enzymes which form a part of a cannabinoid biosynthetic enzyme pathway, and these one or more additional nucleic acid sequences together with the nucleic acid sequence encoding the enzyme can be considered a heterologous nucleic acid sequence. A variety of techniques and methodologies are available and well known in the art for introducing heterologous nucleic acid sequences, such as nucleic acid sequences encoding the cannabinoid pathway enzymes (e.g., AAE, OLS, OAC, and PT), into a host cell so as to attain expression the host cell. The introduction of the heterologous nucleic acids can include integration of the nucleic acids into specific loci (e.g., the NDE1, XII-5, Gal80, ROQ1 loci in yeast) in the genome of a host cell via CRISPR-Cas9 and other techniques, some of which are demonstrated in the Examples herein. Such techniques are well known to the skilled artisan and can, for example, be found in Sambrook and other well-known sources. The number of copies of heterologous pathway genes and their locus of integration in a recombinant host cell's genome can result in improved biosynthetic production of a desired pathway product. Accordingly, it is contemplated that in the recombinant host cells of the present disclosure, the heterologous nucleic acid encoding the recombinant polypeptide having prenyltransferase activity can be integrated in the host cell's genome at one or more loci, including but not limited to the well-known yeast genome loci of NDE1, XII-5, Gal80, ROQ1. In at least one embodiment, the heterologous nucleic acid encoding the prenyltransferase activity (and/or other cannabinoid pathway activities) can be integrated in the host cell genome at two loci selected from: XII-5 and NDE1; or ROQ1 and NDE1.

One of ordinary skill will recognize that the heterologous nucleic acids encoding the recombinant prenyltransferase enzymes and/or other pathway enzymes will further comprise transcriptional promoters capable of controlling expression of the enzymes in the recombinant host cell. Generally, the transcriptional promoters are selected to be compatible with the host cell, so that promoters obtained from bacterial cells are used when a bacterial host cell is selected in accordance herewith, while a fungal promoter is used when a fungal host cell is selected, a plant promoter is used when a plant cell is selected, and so on. Promoters useful in the recombinant host cells of the present disclosure may be constitutive or inducible, provided such promoters are operable in the host cells. Promoters that may be used to control expression in fungal host cells, such as Saccharomyces cerevisiae, are well known in the art and include, but are not limited to: inducible promoters, such as a Gal1 promoter or Gal10 promoter, a constitutive promoter, such as an alcohol dehydrogenase (ADH) promoter, a glyceraldehyde-3-phosphate dehydrogenase (GPD) promoter, or an S. pombe Nmt, or ADH promoter. Exemplary promoters that may be used to control expression in bacterial cells can include the Escherichia coli promoters lac, tac, trc, trp or the T7 promoter. Exemplary promoters that may be used to control expression in plant cells include, for example, a Cauliflower Mosaic Virus 35S promoter (Odell et al. (1985) Nature 313:810-812), a ubiquitin promoter (U.S. Pat. No. 5,510,474; Christensen et al. (1989)), or a rice actin promoter (McElroy et al. (1990) Plant Cell 2:163-171). Exemplary promoters that can be used in mammalian cells include, a viral promoter such as an SV40 promoter or a metallothionine promoter. All of these host cell promoters are well known by and readily available to one of ordinary skill in the art. Further nucleic acid control elements useful for controlling expression in a recombinant host cell can include transcriptional terminators, enhancers, and the like, all of which may be used with the heterologous nucleic acids incorporate in the recombinant host cells of the present disclosure.

A wide variety of techniques are well known in the art for linking transcriptional promoters and other control elements to heterologous nucleic acid sequences encoding cannabinoid pathway genes. Such techniques are described in e.g., Sambrook et al., Molecular Cloning, a Laboratory Manual, Cold Spring Harbor Laboratory Press, 2012, Fourth Ed. Accordingly, in at least one embodiment, the heterologous nucleic acid sequences of the present disclosure comprise a promoter capable of controlling expression in a host cell, wherein the promoter is linked to a nucleic acid sequence encoding a recombinant polypeptide having prenyltransferase activity of the present disclosure, and as necessary, other enzymes constituting a cannabinoid pathway (e.g., AAE, OLS, OAC). This heterologous nucleic acid sequence can be integrated into a recombinant expression vector which ensures good expression in the desired host cell, wherein the expression vector is suitable for expression in a host cell, meaning that the recombinant expression vector comprises the heterologous nucleic acid sequence linked to any genetic elements required to achieve expression in the host cell. Genetic elements that may be included in the expression vector in this regard include a transcriptional termination region, one or more nucleic acid sequences encoding marker genes, one or more origins of replication, and the like. In some embodiments, the expression vector further comprises genetic elements required for the integration of the vector or a portion thereof in the host cell's genome.

It is also contemplated that in some embodiments an expression vector comprising a heterologous nucleic acid of the present disclosure may further contain a marker gene. Marker genes useful in accordance with the present disclosure include any genes that allow the distinction of transformed cells from non-transformed cells, including all selectable and screenable marker genes. A marker gene may be a resistance marker such as an antibiotic resistance marker against, for example, kanamycin or ampicillin. Screenable markers that may be employed to identify transformants through visual inspection include β-glucuronidase (GUS) (U.S. Pat. Nos. 5,268,463 and 5,599,670) and green fluorescent protein (GFP) (Niedz et al., 1995, Plant Cell Rep., 14: 403).

In at least one embodiment, the present disclosure also provides of a method for producing a cannabinoid, wherein a heterologous nucleic acid encoding a recombinant polypeptide having prenyltransferase activity (e.g., an exemplary engineered polypeptide of Table 3) can be introduced into a recombinant host cell. The recombinant host cell can then be used for production of the polypeptide, or incorporated in a biocatalytic process that utilized the prenyltransferase activity of the recombinant polypeptide expressed by the host cell for the catalytic prenylation of a substrate, e.g., the prenylation of OA with GPP to produce CBGA. In at one embodiment, the recombinant host cell can further comprise a pathway of enzymes capable of producing a cannabinoid precursor (e.g., OA or DA) which can act as a substrate for the recombinant polypeptide with prenyltransferase activity. It is contemplated that a recombinant host cell comprising a heterologous nucleic acid encoding a recombinant polypeptide having prenyltransferase activity of the present disclosure can provide improved biosynthesis of a desired cannabinoid (e.g., CBGA) product in terms of titer, yield, and production rate, due to the improved characteristics of the expressed prenyltransferase activity in the cell associated with the amino acid and codon differences engineered in the gene.

Accordingly, in at least one embodiment, the present disclosure provides a method of producing a cannabinoid derivative, wherein the method comprises: (a) culturing in a suitable medium a recombinant host cell of the present disclosure; and (b) recovering the produced cannabinoid derivative. In at least one embodiment, the method of producing a cannabinoid derivative further contacting a cell-free extract of the culture containing the produced cannabinoid with a biocatalytic reagent or chemical reagent capable of converting the cannabinoid to a cannabinoid derivative. In at least one embodiment, the biocatalytic reagent is an enzyme capable of converting the produced cannabinoid to a different cannabinoid or a cannabinoid derivative compound. In at least one embodiment, the chemical reagent is capable of chemically modifying the produced cannabinoid to produce a different cannabinoid or a cannabinoid derivative compound. In at least one embodiment of the method for producing a cannabinoid, the method can further comprise contacting a cell-free extract of the culture containing the produced cannabinoid with a biocatalytic reagent or chemical reagent.

It is contemplated that the cannabinoid, or cannabinoid derivative produced using the methods of the present disclosure can be produced and/or recovered from the reaction in the form of a salt. In at least one embodiment, the recovered salt of the cannabinoid, cannabinoid precursor, cannabinoid precursor derivative, or cannabinoid derivative is a pharmaceutically acceptable salt. Such pharmaceutically acceptable salts retain the biological effectiveness and properties of the free base compound.

It also is contemplated the recombinant polypeptides with prenyltransferase activity of the present disclosure can be incorporated in any biosynthesis method requiring a prenyltransferase catalyzed biocatalytic step. Thus, in at least one embodiment, the recombinant polypeptides having prenyltransferase activity (e.g., exemplary polypeptides of Table 3) can be used in a method for preparing a cannabinoid compound of structural formula (I)

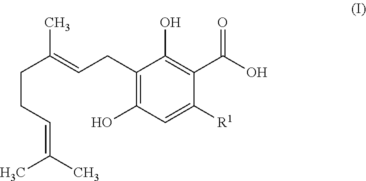

wherein, R¹ is C1-C7 alkyl, wherein the method comprises contacting an recombinant polypeptide having prenyltransferase activity of the present disclosure (e.g., an exemplary recombinant of Table 3) under suitable reactions conditions, with geranyl pyrophosphate (GPP) and a cannabinoid precursor compound of structural formula (II)

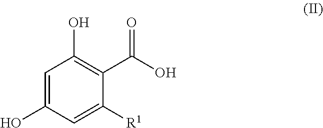

wherein, R¹ is C1-C7 alkyl.

Exemplary conversions of cannabinoid precursor compounds of structural formula (II) to cannabinoid compounds of structural formula (I) that are catalyzed by the recombinant polypeptides having prenyltransferase activity of the present disclosure include: (1) conversion of divarinic acid (DA) to cannabigerovarinic acid (CBGVA); and (2) conversion of olivetolic acid (OA) to cannabigerolic acid (CBGA). It is contemplated that the recombinant polypeptides having prenyltransferase activity of the present disclosure (e.g., polypeptides disclosed in Table 3) can catalyze the conversion of other cannabinoid precursor compounds that are structural analogs of DA and OA, including but not limited to the exemplary cannabinoid precursor compounds listed in Table 2. Accordingly, in at least one embodiment of the biosynthesis method for conversion a cannabinoid precursor compound of structural formula (II) to a cannabinoid compound of structural formula (I), the compound of structural formula (II) is olivetolic acid (OA) and the compound of structure formula (I) is cannabigerolic acid (CBGA). In at least one embodiment, the compound of structural formula (II) is divarinic acid (DA) and the compound of structure formula (I) is cannabigerovarinic acid (CBGVA).

Suitable reaction conditions for the biosynthesis of cannabinoids are known in the art, and can be used with the recombinant polypeptides having prenyltransferase activity of the present disclosure. Additionally, suitable reaction conditions for the exemplary polypeptides of the present disclosure can be determined using routine techniques known in the art for optimizing biocatalytic reactions. It is contemplated that various ranges of suitable reaction conditions with the recombinant polypeptides of the present disclosure, including but not limited to ranges of pH, temperature, buffer, solvent system, substrate loading, polypeptide loading, co-substrate or co-factor loading, atmosphere, and reaction time. Suitable reaction conditions can be readily determined and optimized for particular reactions by routine experimentation that includes, but is not limited to, contacting the recombinant polypeptide and substrate under experimental reaction conditions of concentration, pH, temperature, solvent conditions, and detecting the production of the desired compound of structural formula (I). In at least one embodiment, the suitable reaction conditions comprise a reaction solution of ˜pH 7-8, a temperature of 25 C to 37 C; optionally, the reaction conditions comprise a reaction solution of pH 7 and a temperature of ˜30 C. In at least one embodiment, the reaction solution is allowed to incubate at a temperature of 25 C to 37 C for a reaction time of at least 1, 6, 12, 24, or 48 hours, before the amount of reaction product is determined.

The present disclosure also contemplates that the methods for biocatalytic conversion of a cannabinoid precursor compound of structural formula (II) to a cannabinoid compound of structural formula (I) using an recombinant polypeptide having prenyltransferase activity of the present disclosure can comprise additional chemical or biocatalytic steps carried out on the product compound of structural formula (II), including steps of product compound work-up, extraction, isolation, purification, and/or crystallization, each of which can be carried out under a range of conditions.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Preparation and Screening of Engineered Polypeptides with Improved Prenyltransferase Activity

This example illustrates preparation of site saturation mutagenesis libraries of polypeptides derived from the parent polypeptide, CsdPT4, of SEQ ID NO: 20 and screening for improved activity in the conversion of OA to CBGA relative to the activity of the parent polypeptide of SEQ ID NO: 20.

Materials and Methods

A. Site Saturation Mutagenesis (SSM) Library Build:

The polynucleotide sequence encoding a CsdPT4 polypeptide (SEQ ID NO: 20) from Cannabis sativa was codon optimized as SEQ ID NO: 19 and synthesized as a N-terminal fusion with a gene (SEQ ID NO: 525) encoding the ERG20_{WW} polypeptide (SEQ ID NO: 526). The synthetic gene (SEQ ID NO: 527) encoding the complete ERG20_{WW}-CsdPT4 fusion (SEQ ID NO: 528) was expressed under the Gal1 promoter (SEQ ID NO: 529) and the CYC1 terminator sequence (SEQ ID NO: 530). This synthetic gene was integrated as a knock-in using CRISPR-Cas9 at the NDE1 site in a parent yeast strain, which already had integrated genes encoding the cannabinoid pathway enzyme activities of AAE, OLS, and OAC. The resulting strain, EVP001, integrated with the cannabinoid pathway and the ERG20_{WW}-CsdPT4 gene was used as a control strain in screening the saturation mutagenesis library strains for fold-improvement in CBGA titer as described below. A further screening strain was built by integrating the m-Venus cassette as a N-terminal fusion with the ERG20_{WW} gene encoding the ERG20_{WW}-m-Venus polypeptide at the NDE1 site expressed under the Gal1 promoter (SEQ ID NO: 529) and the CYC1 terminator sequence (SEQ ID NO: 530), thereby replacing the previously integrated CsdPT4 gene (SEQ ID NO: 19). This resulting EVP000 strain was no longer capable of converting OA to CBGA.

Genomic DNA from a strain, with the ERG20_{WW}-CsdPT4 fusion integrated at NDE1 site (EVP001), was used as the template to generate two PCR products: (1) a first PCR product (Fragment A), which does not harbor any degenerate codons, and (2) a second PCR product (Fragment B), which has sequence overlap with the Fragment A, and is amplified harboring one NNK degenerate codon only. Primers used for amplification of Fragments A and B and overlap extension were designed according to standard site-saturation mutagenesis protocols. Fragment B was amplified with a series of forward primers that included the single NNK degenerate codon scanned across the various desired positions and a single reverse primer of SEQ ID NO: 532. Fragment A was amplified using a single forward primer of SEQ ID NO: 533 and a series of reverse primers designed according to the location of the mutagenesis site. The two fragments A and B were further assembled by overlap extension PCR using forward primer of SEQ ID NO: 534 and reverse primer of SEQ ID NO: 535. The assembled OE-PCR products were then pooled, and gel purified to provide a saturation mutagenesis library of linear donor DNA.

The pooled saturation mutagenesis library of linear donor DNA was transformed in a yeast strain, EVP000, for screening which, like EVP001, already had integrated genes encoding the cannabinoid pathway enzyme activities of AAE, OLS, and OAC. The library of linear donor DNA was integrated in EVP000 as a knock-in using CRISPR-Cas9 to replace the m-Venus cassette having an ORF of SEQ ID NO: 531 located at the NDE1 site under control the Gal1 promoter and CYC1 terminator.

B. Screening of Site Saturation Mutagenesis Library for Cannabinoid Biosynthesis:

Individual clones from the saturation mutagenesis library integrated into EVP000, and the EVP001 control strain were picked and grown in 0.3 mL YPD in 96-well plates. The culture plates were incubated in shaking incubators for 48 h at 30 C, 85% humidity, and 250 rpm. Cultures were then sub-cultured into 0.27 mL fresh YPD and fed with hexanoic acid (HA) to 2 mM final concentration. Subculture plates were grown in shaking incubators for 48 hours at 30 C, 85% humidity, and 250 rpm. The whole broth from these sub-culture plates was extracted and analyzed for the presence of the cannabinoid precursor compound, OA, and the cannabinoid product, CBGA, using HPLC, as described below.

1. HPLC sample preparation: The whole broth of the culture was extracted and diluted with MeOH for sample preparation. The prepared samples were loaded onto RapidFire365 coupled with a triple quadruple mass spectrometry detector. Metabolites OA and CBGA were detected using MRM mode. Calibration curves of OA and CBGA were generated by running serial dilutions of standards, and then used to calculate concentrations of each metabolite.

2. HPLC instrumentation and parameters: HPLC system: Agilent RapidFire 365; Column: Agilent Cartridge C18 (12 μl, type C); Mobile phase: Pump 1 uses 95:5 H₂O:acetonitrile with 0.1% formic acid at 1 mL/min; Pump 2 uses 20:80 acetonitrile:H₂O at 0.8 mL/min; Pump 3 uses MeOH with 0.1% formic acid at 0.8 mL/min; Aqueous wash uses H₂O; Organic wash uses acetonitrile; RapidFire cycle time: Aspiration 600 ms; Load/wash 3000 ms; Extra wash 2000 ms; Elute 4000 ms; Re-equilibration 500 ms.

C. Sequencing

Those clones from the saturation mutagenesis library determined by screening to exhibit an increased CBGA titer compared to the control, were re-tested and sequenced using Sanger sequencing technology to determine the respective specific codon and amino acid differences.

D. Results

Screening data from the saturation mutagenesis library strains in terms of fold-improvement in production of CBGA titer from HA feeding (FIOPC), relative to the control strain, EVP001, which expresses the parent CsdPT4 polypeptide of SEQ ID NO: 20, are summarized in Table 5 (below).

TABLE 5

| NT | AA | AA Substitution and/or | FIOPC |
|---|---|---|---|
| SEQ | SEQ | NT Codon Change | (relative |
| ID NO: | ID NO: | (relative to CsdPT4) | to CsdPT4) |
| 19 | 20 | n/a | 1.0 |
| 21 | 22 | F134G (TTT > GGG), | 3.83 |
| | | S175V (TCT > GTG) | |
| 23 | 24 | I79C | 2.04 |
| 25 | 26 | E106R (GAA > CGG), | 1.26 |
| | | A182 (GCA > GCC) | |
| 27 | 28 | W61A | 1.06 |
| 29 | 30 | S175V (TCT > GTT) | 2.19 |
| 31 | 32 | G58S, F73 (TTT > TTC) | 1.45 |
| 33 | 34 | W61V | 1.29-1.47 |
| 35 | 36 | F64M | 1.25-1.30 |
| 37 | 38 | F64L | 1.27 |
| 39 | 40 | F134G (TTT > GGT) | 1.19 |
| 41 | 42 | I79A (ATC > GCT) | 2.88 |
| 43 | 44 | S177A | 1.9 |
| 45 | 46 | F173I | 1.26 |
| 47 | 48 | W153L (TGG > TTG) | 2.80 |
| 49 | 50 | F64G | 1.14 |
| 51 | 52 | I79S | 1.02 |
| 53 | 54 | G119 (GGG > GGT) | 0.87 |
| 55 | 56 | R152 (AGA > CGT) | 1.36 |
| 57 | 58 | G139 (GGT > GGG) | 1.72 |
| | | S175V (TCT > GTG) | |
| 59 | 60 | M80V | 1.26 |
| 61 | 62 | I79A (ATC > GCG) | 2.07 |
| 63 | 64 | S181R | 1.42 |
| 65 | 66 | S177G (TCA > GGT) | 1.41 |
| 67 | 68 | I113W | 1.72 |
| 69 | 70 | F134G (TTT > GGG) | 1.12 |
| 71 | 72 | E106R (GAA > CGG) | 1.28 |
| 73 | 74 | W153L (TGG > CTG) | 2.50 |
| 75 | 76 | T180R | 2.67 |
| 77 | 78 | S175T | 1.91 |
| 79 | 80 | R46K, F64T | 3.19 |
| 81 | 82 | S177G (TCA > GGG) | 1.34 |
| 83 | 84 | F132F (TTC > TTT) | 1.92 |
| 85 | 86 | I165L | 1.52 |
| 87 | 88 | T180V | 1.52 |
| 89 | 90 | F75W | 1.67 |
| 91 | 92 | S177T | 2.13 |
| 93 | 94 | T180L | 0.57 |
| 95 | 96 | A293G | 0.94 |
| 97 | 98 | N235C | 1.73 |
| 99 | 100 | F161V (TTC > GTT), A293V | 1.52 |
| 101 | 102 | F158G (TTT > GGG) | 1.27 |
| 103 | 104 | S295A | 1.09-4.02 |
| 105 | 106 | E284R (GAA > CGG) | 1.3-2.15 |
| 107 | 108 | N50D, E284R (GAA > AGG) | 1.21-1.25 |
| 109 | 110 | V99A (GTT > GCG) | 1.37 |
| 111 | 112 | E284D | 1.03 |
| 113 | 114 | E284K (GAA > AAA), A291E | 2.64 |
| 115 | 116 | P294E | 1.15 |
| 117 | 118 | E284R (GAA > AGG) | 1.88 |
| 119 | 120 | Q82 (CAA > CAG) | 5.82 |
| 121 | 122 | A293K | 1.15 |
| 123 | 124 | D87E, V99A (GTT > GCG) | 1.32 |
| 125 | 126 | N235V | 1.38 |
| 127 | 128 | P97 (CCA > CCG) | 0.79 |
| 129 | 130 | F161V (TTC > GTT) | 1.70 |
| 131 | 132 | F158G (TTT > GGG) | 1.41-1.64 |
| 133 | 134 | E284K (GAA > AAG) | 2.31 |
| 135 | 136 | T229V | 0.90 |
| 137 | 138 | N235K | 1.27 |

As shown by the results in Table 5, the presence of the following amino acid differences in the recombinant polypeptides having prenyltransferase activity expressed in the strains from the EVP000 saturation mutagenesis libraries resulted in increased CBGA titer produced by the yeast strain: R46K, N50D, G58S, W61A, W61V, F64G, F64L, F64M, F64T, F75W, I179A, I79C, I79S, M80V, D87E, V99A, E106R, I113W, F134G, W153L, F158G, F161V, I165L, F173I, S175T/V, S177A, S177G, S177T, T180L, T180R, T180V, S181R, T229V, N235C, N235K, N235V, E284D, E284K, E284R, A291E, A293G, A293K, A293V, P294E, and S295A. Additionally, at least the following combinations of residue differences in the expressed recombinant polypeptides resulted in increased CBGA titer produced by the yeast strain: R46K and F64T; N50D and E284R; D87E and V99A; F134G and S175V; F161V and A293V; and E284K and A291E.

It also was observed that certain neutral (silent), codon changes, which did not result in an amino acid change in the recombinant polypeptide sequence, resulted in increased CBGA titer produced by the yeast strain. Specifically, the following codon differences at positions F73, Q82, P97, G119, F132, G139, R152, and A182: F73 (TTT>TTC); Q82 (CAA>CAG); P97 (CCA>CCG); G119 (GGG>GGT); F132F (TTC>TTT); G139 (GGT>GGG); R152 (AGA>CGT); and A182 (GCA>GCC).

### Example 2: Preparation and Screening of Engineered Polypeptides with Improved Prenyltransferase Activity

This example illustrates preparation of combinatorial mutagenesis libraries of polypeptides derived from the parent polypeptide, CsdPT4, of SEQ ID NO: 20 using both semi-synthetic and synthetic approaches, and screening for improved activity in the conversion of OA to CBGA relative to the activity of the parent polypeptide of SEQ ID NO: 20.

Materials and Methods

A. Combinatorial Library Builds:

The polynucleotide sequence encoding a CsdPT4 polypeptide (SEQ ID NO: 20) from Cannabis sativa was codon optimized as SEQ ID NO: 19 and synthesized as a N-terminal fusion with a gene (SEQ ID NO: 525) encoding the ERG20_{WW} polypeptide (SEQ ID NO: 526). The resulting synthetic gene (SEQ ID NO: 527) encoding the complete ERG20_{WW}-CsdPT4 fusion (SEQ ID NO: 528) was expressed under the Gal1 promoter (SEQ ID NO: 529) and the CYC1 terminator sequence (SEQ ID NO: 530). This synthetic gene was integrated as a knock-in using CRISPR-Cas9 at the NDE1 site in a parent yeast strain, which already had integrated genes encoding the cannabinoid pathway enzyme activities of AAE, OLS, and OAC. The resulting strain, EVP001, integrated with the cannabinoid pathway and the ERG20_{WW}-CsdPT4 gene was used as a control strain in screening the combinatorial mutagenesis library strains for fold-improvement in CBGA titer as described below. A further screening strain was built by integrating the m-Venus cassette as a N-terminal fusion with the ERG20_{WW}, encoding the ERG20_{WW}-m-Venus polypeptide at the Nde1 site expressed under the Gal1 promoter (SEQ ID NO: 529) and the CYC1 terminator sequence (SEQ ID NO: 530), thereby replacing the previously integrated CsdPT4 gene (SEQ ID NO: 19). This resulting EVP000 strain was no longer capable of converting OA to CBGA.

Semi-synthetic approach: A semi synthetic approach was used to construct the first set of combinatorial libraries. Genomic DNA from a strain with the ERG20_{WW}-CsdPT4 fusion integrated at NDE1 site (EVP001), was used as the template to generate a PCR amplicon of CsdPT4 containing uracil using a dNTP mix comprising of the following deoxyribonucleotides: dATP, dGTP, dCTP, dTTP, dUTP. The resulting PCR product was gel purified and digested with Uracil-DNA Glycosylase and Endonuclease IV at 37 C for 2 hours, followed by 94 C for two minutes, to generate a pool of fragments in the range of 50-100 bases. These fragments were further combined with seven individual pools of synthesized oligonucleotides (up to 60 bases in length, containing one single amino acid change per oligo) in seven individual assembly PCR reactions to reassemble the full-length PCR product and incorporate mutagenic amino acid changes within each pool randomly. The combinatorial library was prepared by individually amplifying the seven assembled products using overlap extension PCR using forward primer of SEQ ID NO: 536 and reverse primer of SEQ ID NO:537. The seven pools of oligonucleotides are summarized in Table 6 below.

TABLE 6

| Pool | Oligo Sequences |
|---|---|
| #1 | SEQ ID NO: 538-562 |
| #2 | SEQ ID NO: 563-587 |
| #3 | SEQ ID NO: 588-612 |
| #4 | SEQ ID NO: 613-647 |
| #5 | SEQ ID NO: 648-681 |
| #6 | SEQ ID NO: 682-705 |
| #7 | SEQ ID NO: 706-723 |

The seven resulting PCR products were further pooled to prepare a single semi-synthetic combinatorial library of linear donor DNA.

Fully synthetic approach: A second, fully synthetic combinatorial library was designed using positions and mutations identified from the initial SSM screening described in Example 1. Amino acids positions in the original SSM screens where more than one amino acid mutation was identified to increase titer were included in the design. The final combinatorial library was synthesized to include combinations of the amino acid changes at 19 positions as summarized in Table 7 below.

TABLE 7

| Wild-type AA Position | Amino Acid Mutations |
|---|---|
| P5 | G, V |
| D10 | V, L, W |
| C41 | A, G, S |
| F49 | L, R, M |
| W61 | A, V |
| F64 | M, L, G, T, W |
| I79 | C, A |
| K125 | M, V, W |
| F158 | G, A |
| S175 | V, T, A, G |
| S177 | A, G, T |
| T180 | R, V, L |
| R190 | S, G, A, Q |
| S194 | V, A, L |
| N235 | C, V, K |
| F238 | W, L |
| C277 | M, A |
| E284 | R, D, K |
| A293 | G, V, K |

The following sequences were also synthesized at the 5′ and 3′ ends of the library to facilitate overlap and extension PCR to include homology sequences to facilitate integration:

5′ additional sequence:
(SEQ ID NO: 724)
AAGTTTACAAGAGAAGCAAAGGTAGCGGCAGCGGTAGCGGTAGCGGCAGC.
3′ additional sequence:
(SEQ ID NO: 725)
TGATCATGTAATTAGTTATGTCACGCTTACATTCACGCCCTCCCCCCACA.

The resulting combinatorial variants were pooled to prepare a single synthetic combinatorial library of linear donor DNA.

The pooled semi-synthetic and synthetic combinatorial libraries of linear donor DNA were transformed in a yeast strain, EVP000, which, like EVP001, already had integrated genes encoding the cannabinoid pathway enzyme activities of AAE, OLS, and OAC. The library of linear donor DNA was integrated in EVP000 as a knock-in using CRISPR-Cas9 to replace an m-Venus cassette having an ORF of SEQ ID NO: 531 located at the NDE1 site under control the Gal1 promoter and CYC1 terminator.

B. Screening of Semi-Synthetic and Synthetic Combinatorial Libraries for Cannabinoid Biosynthesis:

Individual clones from both the semi-synthetic combinatorial library and the synthetic combinatorial library integrated into EVP000, along with the EVP001 control strain were picked and grown in 0.3 mL YPD in 96-well plates. The culture plates were incubated in shaking incubators for 48 h at 30 C, 85% humidity, and 250 rpm. Cultures were then sub-cultured into 0.27 mL fresh YPD and fed with hexanoic acid (HA) to 2 mM final concentration. Subculture plates were grown in shaking incubators for 48 hours at 30 C, 85% humidity, and 250 rpm. The whole broth from these sub-culture plates was extracted and analyzed for the presence of the cannabinoid precursor compound, OA, and the cannabinoid product, CBGA, using HPLC, as described below.

1. HPLC sample preparation: The whole broth of the culture was extracted and diluted with MeOH for sample preparation. The prepared samples were loaded onto RapidFire365 coupled with a triple quadruple mass spectrometry detector. Metabolites OA and CBGA were detected using MRM mode. Calibration curves of OA and CBGA were generated by running serial dilutions of standards, and then used to calculate concentrations of each metabolite.

2. HPLC instrumentation and parameters: HPLC system: Agilent RapidFire 365; Column: Agilent Cartridge C18 (12 μl, type C); Mobile phase: Pump 1 uses 95:5 H2O:acetonitrile with 0.1% formic acid at 1 mL/min; Pump 2 uses 20:80 acetonitrile:H₂O at 0.8 mL/min; Pump 3 uses MeOH with 0.1% formic acid at 0.8 mL/min; Aqueous wash uses H₂O; Organic wash uses acetonitrile; RapidFire cycle time: Aspiration 600 ms; Load/wash 3000 ms; Extra wash 2000 ms; Elute 4000 ms; Re-equilibration 500 ms.

C. Sequencing

Those clones from each of the combinatorial libraries (semi-synthetic and fully synthetic) determined by screening to exhibit an increased CBGA titer compared to the control EVP001, were re-tested and sequenced using Sanger sequencing technology to determine the specific codon and amino acid differences.

D. Results

Strains were identified from screening the semi-synthetic and fully synthetic combinatorial libraries for fold-improvement in CBGA titer from HA feeding (FIOPC) relative to the control strain, EVP001, which expresses the parent CsdPT4 polypeptide of SEQ ID NO: 20. The engineered prenyltransferase polypeptides expressed in these improved strains, along with their specific amino acid substitutions and/or silent codon changes are summarized below in Tables 8 and 9, respectively.

TABLE 8

| NT SEQ | AA SEQ | AA Substitutions | Silent Codon | |
|---|---|---|---|---|
| ID NO: | ID NO: | (relative to CsdPT4) | Changes | FIOPC |
| 139 | 140 | P5G, H7C, C41S, F64T, F134G, S175V, S177A, | | 1.68 |
| | | G204S, L249A, S295A | | |
| 141 | 142 | H7C, D10V, C41A, R46K, F64T, I79C, K125W, | | 1.66 |
| | | F134G | | |
| 143 | 144 | H7C, D10V, I79C, F134G, S175T, S177A, T180R, | N160 | 1.63 |
| | | R190S, G204S | (AAT > AAC) | |
| 145 | 146 | P5G, H7C, D10V, T180R, G204S, S241F | 1213 | 1.56 |
| | | | (ATC > ATT) | |
| 147 | 148 | D10V, C41S, F64T, I79C, W153L, S175T, T180R, | L304 | 1.56 |
| | | G204S, L249A, C277M, F280R, Q281R, A291E, | (TTG > CTG) | |
| | | S295A, Y307H, A308E, E309V, Y310S | | |
| 149 | 150 | P5G, H7C, C41A, F64T, I79A, I113N, W153L, | | 1.52 |
| | | S175V, T180R, S194L, I197T, G204S | | |
| 151 | 152 | P5G, H7C, D10V, F64T, W153L, S175V, V188A, | | 1.49 |
| | | R190S | | |
| 153 | 154 | H7C, R46K, I79C, K125W, F134G, Y176S, S177A, | V224 | 1.48 |
| | | T180R, G204S, C277A, L278P, F280G, Q281R, | (GTT > GTC) | |
| | | T282P | | |
| 155 | 156 | H7C, C41S, R46K, I79C, K125W, S175T, S177T, | | 1.48 |
| | | T180R, R190S, G204S, S251A, C277M, Q281R, | | |
| | | A291E | | |
| 157 | 158 | P5G, C41A, K125W, W153L, S175T, S177A, | | 1.46 |
| | | T180R | | |
| 159 | 160 | H7C, S177T, T180R, S194V, G204A, S295A | | 1.45 |
| 161 | 162 | P5G, H7C, C41A, F64T, K125W, F134G, S177A, | F311 | 1.45 |
| | | G204S, C277A, F280R, F301S | (TTT > TTC) | |
| 163 | 164 | D10V, C41S, R46K, F134G, W153L, S177T, | | 1.44 |
| | | T180R, V188A, R190S, M205G, L249A, C277M, | | |
| | | F280R | | |
| 165 | 166 | P5G, H7C, D10V, F49L, R52P, K125W, W153L, | | 1.44 |
| | | S175V, S177T, T180R, S194L, G204S, M205G | | |
| 167 | 168 | H7C, D10V, C41A, R46K, R52P, S175V, S177A, | | 1.44 |
| | | T180R, V188A, G204S, M205G | | |
| 169 | 170 | P5G, H7C, I79C, F134G, W153L, S175V | | 1.43 |
| 171 | 172 | D10V, C41S, R46K, F134G, W153L, S175V, | | 1.42 |
| | | G204S | | |
| 173 | 174 | H7C, R46K, I79A, S177A, T180R, V188A, G204S | S166 | 1.41 |
| | | | (TCT > TCC) | |
| 175 | 176 | H7C, D10V, C41A, K125W, W153L, S175T, | T201 | 1.41 |
| | | V188S, R190S, M200R, M205G, S214C, D219V, | (ACT > ACG), | |
| | | V243A, S251C, S264Y, Q281R, A288P | G218 | |
| | | | (GGT > GGG), | |
| | | | G242 | |
| | | | (GGT > GGC), | |
| | | | F276 | |
| | | | (TTC > TTT) | |
| 177 | 178 | H7C, K125W, S175V, S177T, T180R, S194L, | Y306 | 1.4 |
| | | G204S, S251A, S295A | (TAC > TAT) | |
| 179 | 180 | H7C, D10V, C41A, R46K, V68D, I79A, W153L, | | 1.38 |
| | | S175V, S177T, T180R, V188A, R190S, G204S, | | |
| | | M205G | | |
| 181 | 182 | P5G, R46K, R52P, F64T, L249A, E284R, A291E, | G119 | 1.37 |
| | | S295A | (GGT > GGC) | |
| 183 | 184 | P5G, H7C, C41S, R46K, K125W, F134G, I165T, | Y306 | 1.37 |
| | | S175T, S177T, T180R, G204S, Q281R, S295A | (TAT > TAC) | |
| 185 | 186 | H7C, D10V, C41A, F64T, W153L, S175V, S177A, | | 1.37 |
| | | T180R, M205G | | |
| 187 | 188 | P5G, C41S, R52P, I79C | | 1.37 |
| 189 | 190 | P5G, H7C, R52P, I79A, F134G, W153L, G204S, | | 1.37 |
| | | M205G, L249A, C277A, F280R, Q281R, A291E | | |
| 191 | 192 | D10V, R46K, W153L, T180R, S194V, L249A, | V261 | 1.36 |
| | | C277M, F280R, Q281R, A291E, S295A | (GTT > GTC) | |
| 193 | 194 | D10V, C41S, K125W, F134G, S175V, S177A, | | 1.36 |
| | | T180R | | |
| 195 | 196 | P5G, D10V, C41S, K125W, F134G, T180R | | 1.36 |
| 197 | 198 | H7C, I79A, K125W, W153L, S175T, T180R, | | 1.36 |
| | | R190S, M205G, L249A, S251A, C277A, F280R, | | |
| | | A291E | | |
| 199 | 200 | P5G, H7C, D10V, C41S, R46K, I121T, K125W, | | 1.35 |
| | | F134G, W153L, S175V, S177T, T180R, V188A, | | |
| | | R190S, M205G | | |
| 201 | 202 | P5G, C41S, S175V, S177A, T180R, L249A, C277A | | 1.35 |
| 203 | 204 | H7C, D10V, F49L, I79C, W153L, S175V, S177T, | | 1.34 |
| | | T180R, V188A, S194L | | |
| 205 | 206 | P5G, H7C, C41S, F49L, R52P, F64T, I79C, | V133 | 1.34 |
| | | K125W, W153L, S175T, S177T, T180R, V188A, | (GTT > GTC) | |
| | | R190S, G204S, C277M, A291E | | |
| 207 | 208 | P5G, H7C, D10V, F64T, F134G, W153L, S177T, | | 1.34 |
| | | T180R, L249A, C277A, Q281R, A291E, S295A | | |
| 209 | 210 | H7C, D10V, I79C, S177A, T180R, V188A, R190S, | V99 | 1.33 |
| | | A291E, S295A | (GTT > GTC) | |
| 211 | 212 | H7C, D10V, C41S, R46K, I79A, K125W, S175V, | | 1.33 |
| | | T180R, R190S | | |
| 213 | 214 | P5G, H7C, D10V, I79C, K125W, S175V, S177T, | | 1.32 |
| | | R190S, G204S, M205G, L249A, S251A, C277M, | | |
| | | A291E | | |
| 215 | 216 | D10V, R46K, K125W, W153L, S175V, S177A, | | 1.31 |
| | | T180R, V188A, C277M, S295A | | |
| 217 | 218 | P5G, H7C, C41S, F64T, K125W, F134G, S175V, | | 1.31 |
| | | S177A, V188A, M205G | | |
| 219 | 220 | P5G, C41A, R46K, F134G, S175T, T180R, V188A, | Q155 | 1.31 |
| | | R190S | (CAA > CAG) | |
| 221 | 222 | H7C, D10V, C41A, R52P, K125W, F134G, S177T, | L111 | 1.31 |
| | | S194V | (TTA > TTG) | |
| 223 | 224 | P5G, D10V, R52P, I79C, K125W, W153L | | 1.31 |
| 225 | 226 | P5G, H7C, D10V, F49L, K125W, F134G, W153L, | | 1.29 |
| | | S194L, G204S, Q281R, S295A | | |
| 227 | 228 | P5G, R52P, F64T, I79C, D95N | K93 | 1.29 |
| | | | (AAG > AAA) | |
| 229 | 230 | P5G, H7C, I79A, I105V, S177A, T180R, S194V, | | 1.28 |
| | | M205G, Q281R, S295A, V314A | | |
| 231 | 232 | D10V, C41S, R46K, R52P, F64T, F134G, S177A, | | 1.28 |
| | | T180R | | |
| 233 | 234 | P5G, D10V, R46K, R52P, F64T, I79C, K125W, | | 1.28 |
| | | W153L, T180R | | |
| 235 | 236 | H7C, C41S, R52P, I79C, K125W, F134G, W153L, | | 1.28 |
| | | S175T, T180I, V188A, R190S, L249A, S251A, | | |
| | | F280R, Q281R, S295A | | |
| 237 | 238 | H7C, D10V, K125W, F134G, W153L, S175T, | V33 | 1.27 |
| | | S177T | (GTT > GTC) | |
| 239 | 240 | P5G, C41S, R46K, K125W, F134G, W153L, | | 1.27 |
| | | S175T, S177A, S194V | | |
| 241 | 242 | P5G, D10V, I79A, K125W, F134G, S175V, C277M, | | 1.26 |
| | | Q281R, A291E, S295A | | |
| 243 | 244 | P5G, D10V, C41A, R46K, R52P, F134G, W153L, | A78 | 1.26 |
| | | S175T, S177T, M205G, L249A, S251A, F280R, | (GCA > GCG) | |
| | | Q281R, A291E | | |
| 245 | 246 | P5G, C41S, I79C, W153L, S175T, M205G, L249A, | | 1.26 |
| | | S251A, C277A, F280R, A291E, Y313P | | |
| 247 | 248 | P5G, H7C, D10V, C41A, R52P, T123K, K125W | | 1.25 |
| 249 | 250 | P5G, D10V, C41A, R46K, K125W, F134G, W153L, | | 1.24 |
| | | S175V, S177T, T180R, G204S, M205G, S253P, | | |
| | | F280R, A291E, S295A, F315S | | |
| 251 | 252 | H7C, C41A, F49L, I79C, K125W, W153L, S177T | | 1.24 |
| 253 | 254 | P5G, H7C, C41A, S177A, T180R, M205G, L249A, | | 1.24 |
| | | S251A, C277A, A291E, S295A, F299L | | |
| 255 | 256 | H7C, D10V, C41S, F64T, S177A, V188A, M205G, | | 1.23 |
| | | L249A, S251A, A291E, S295A | | |
| 257 | 258 | H7C, D10V, R46K, F134V | | 1.23 |
| 259 | 260 | P5G, H7C, F49L, F64T, I79C, K125W, W153L, | | 1.23 |
| | | S175T, S194L, C277M, F280R, Q281R, A291E, | | |
| | | S295A | | |
| 261 | 262 | P5G, D10V, F64T, F134G, S175T, S177T, T180R, | | 1.23 |
| | | L249A, S251A, A291E | | |
| 263 | 264 | P5G, H7C, D10V, I79A, K125W, W153L, E284K, | K263 | 1.22 |
| | | A291E | (AAA > AAG) | |
| 265 | 266 | P5G, D10V, F49L, R52P, F134G, S194L, S251A, | | 1.21 |
| | | W258R, Q281R, A291E, S295A | | |
| 267 | 268 | H7C, D10V, C41S, I79C, F134G, V188A, R190S, | Y306 | 1.21 |
| | | S214C, A291E, S295A, F311S | (TAT > TAC), | |
| | | | V312 | |
| | | | (GTT > GTC) | |
| 269 | 270 | C41A, I79A, K125W, F134G, W153L, S175T, | | 1.21 |
| | | S177T, T180R | | |
| 271 | 272 | P5G, H7C, I79A, S175V, S177T, L249A, S251A, | | 1.2 |
| | | W258R, Q281R | | |
| 273 | 274 | H7C, D10V, C41A, R46K, F64T, V115A, K125W, | | 1.2 |
| | | T180R, C277A, F280R, S295A | | |
| 275 | 276 | D10V, C41A, R46K, R52P, F64T, K125W, F134G, | L111 | 1.2 |
| | | W153L, S177A, T180R, S214C, L249A, E284K, | (TTA > TTG), | |
| | | S295A, A308P | Y306 | |
| | | | (TAT > TAC) | |
| 277 | 278 | P5G, D10V, C41A, R46K, I79A, F134G, W153L, | | 1.2 |
| | | L249A, Q281R, S295A | | |
| 279 | 280 | P5G, H7C, R46K, K125W, S175V, S177T, T180R, | | 1.2 |
| | | M205G, L249A, E284K, S295A | | |
| 281 | 282 | D10V, C41S, R46K, F65L, K125W, W153L, S177A, | | 1.18 |
| | | T180R, S194L, L249A, S251A, C277A | | |
| 283 | 284 | C41A, R46K, I79C, K93N, K125W | | 1.18 |
| 285 | 286 | P5G, H7C, R52P, I79C, K125W, W153L, S177T, | | 1.16 |
| | | T180R, S194L | | |
| 287 | 288 | C41A, R46K, W153L, S175V, M205G | | 1.16 |
| 289 | 290 | H7C, R52P, W153L, S175V, G204S, M205G, | | 1.15 |
| | | C277M, Q281R, S295A | | |
| 291 | 292 | P5G, R52P, I79A, K125W | | 1.14 |
| 293 | 294 | P5G, H7C, D10V, C41S, F49L, R52P, F64T, | N160 | 1.14 |
| | | S175V, S177A, M205G | (AAT > AAC) | |
| 295 | 296 | P5G, F49L, I79A, S177T | | 1.14 |
| 297 | 298 | P5G, D10V, C41S, R46K, I79N, F134G, S175V, | | 1.13 |
| | | S177T, T180R, M205G, A291E, S295A | | |
| 299 | 300 | H7C, D10V, R46K, K125W, F158S, S175V, S177T, | | 1.13 |
| | | T180R, V188A, R190S, M205R, L249A, C277R, | | |
| | | F280R, Q281R, A291E, Y310S | | |
| 301 | 302 | P5G, H7C, C41A, R52P, F134G, W153L, S175T, | S225 | 1.11 |
| | | L249A, S251A, E284R, S295A, L305S | (TCA > TCG) | |
| 303 | 304 | H7C, C41S, R46K, K125W, S194L, Q281R, A291E | Y306 | 1.11 |
| | | | (TAT > TAC) | |
| 305 | 306 | H7C, F49L, K125W, F134G, S177T, M205G, | Y306 | 1.1 |
| | | E284R, A291E, S295A | (TAT > TAC) | |
| 307 | 308 | P5G, H7C, D10V, C41A, R46K, F134G, F144S, | S104 | 1.09 |
| | | W153L, G204S, L249A, S251A, C277M, F280R, | (TCA > TCT) | |
| | | Q281R | | |
| 309 | 310 | P5G, R46K, L54S, K125W, W153L, S175T, S177T, | Y306 | 1.09 |
| | | S214C, F276L, F280R, Q281R, A308P, Y310C, | (TAT > TAC) | |
| | | Y313H | | |
| 311 | 312 | P5G, D10V, R46K, I79C | | 1.07 |
| 313 | 314 | P5G, D10V, R46K, W153L, S175V, S177T, T180R, | Y306 | 1.07 |
| | | S214C, S251A, C277M, F280R, Q281R, S295A, | (TAT > TAC) | |
| | | F301S, I302L, W303C, L304R, Y310S, F311S | | |
| 315 | 316 | D10V, R46K, F64T, I79A, W153L, S177A, T180R, | L117 | 1.07 |
| | | S194L, S251A, S295A | (TTG > CTG) | |
| 317 | 318 | H7C, D10V, C41A, S175V, F193L | | 1.06 |
| 319 | 320 | H7C, C41A, R46K, R52P, K125W, F134G, S177T | | 1.06 |
| 321 | 322 | P5G, H7C, D10V, C41A, K125W, S194L, G204S, | L162 | 1.06 |
| | | M205G, F280R, A291E, S295A | (TTG > CTG), | |
| | | | Y306 | |
| | | | (TAT > TAC) | |
| 323 | 324 | P5G, F49L, R52P, K125W, F134G, W153L, S177T, | Y306 | 1.06 |
| | | R190S, M205G, S214C, F280R, A291E, S295A, | (TAT > TAC) | |
| | | V312G, Y313H | | |
| 325 | 326 | H7C, D10V, K125W, F134G | | 1.06 |
| 327 | 328 | D10V, F49L, R52P, F64T, W153L, S175V, S177A, | N74 | 1.06 |
| | | Q281R, S295A, F311P | (AAT > AAC), | |
| | | | Y306 | |
| | | | (TAT > TAC) | |
| 329 | 330 | C41S | | 1.05 |
| 331 | 332 | H7C, D10V, C41S, R46K, K125W, W153L, S194A | | 1.05 |
| 333 | 334 | R52P, F64T, I79C, F134G, S177A, T180R, L249A, | I37 | 1.05 |
| | | M267T, C277M, Q281R, L287F, A288P, Y290S | (ATT > ATC), | |
| | | | A233 | |
| | | | (GCA > GCG) | |
| 335 | 336 | D10V, F64T, I79C, K125W, F134G, I140T, S177A, | Y306 | 1.04 |
| | | L249A, C277M, Q281R, A291E | (TAT > TAC) | |
| 337 | 338 | D10V, K34E, F49L | | 1.04 |
| 339 | 340 | H7C, D10V, C41A, R46K, F64T, K125W, R190S, | L304 | 1.03 |
| | | M205G, C277A, S295A, Y307S, A308R, Y310S | (TTG > CTG), | |
| | | | Y306 | |
| | | | (TAT > TAC) | |
| 341 | 342 | P5G, H7C, D10V, C41G, K125W, F134G, L249V, | S295 | 1.02 |
| | | F280L, A291E | (TCA > TCT) | |
| 343 | 344 | P5G, F64T, I79C, W153L, I165T, Q281R, A291E, | | 1.02 |
| | | S295A | | |
| 345 | 346 | H7C, R46K, F64T, I91V, W153L, S175V, S177T, | Y306 | 1.02 |
| | | I196T, M205G, L249A, C277M, A291E, Y310P | (TAT > TAC) | |
| 347 | 348 | H7C, C41A, K125W, F134G, W153L, S175T, | | 1 |
| | | T180R, R190S, M205G, E217G | | |

TABLE 9

| NT SEQ | AA SEQ | AA Substitutions | Silent Codon | |
|---|---|---|---|---|
| ID NO: | ID NO: | (relative to CsdPT4) | Changes | FIOPC |
| 349 | 350 | P5G, D10W, C41G, F49M, W61A, F64W, I79A, | | 2.07 |
| | | K125M, F158G, S175A, S177A, T180L, R190S, | | |
| | | S194V, N235K, F238W, C277A, E284D, A293V | | |
| 351 | 352 | P5G, D10V, C41S, F49M, W61A, F64L, I79C, | | 2.01 |
| | | K125V, F158G, S175V, S177A, T180V, R190S, | | |
| | | S194V, N235C, F238L, C277A, E284K, A293K | | |
| 353 | 354 | P5G, D10V, C41A, F49L, W61A, F64G, I79C, | | 1.98 |
| | | K125V, F158G, S175A, S177T, T180V, R190S, | | |
| | | S194V, N235K, F238W, C277A, E284D, A293G | | |
| 355 | 356 | C41A, F49L, W61V, F64T, I79A, K125M, F158G, | | 1.95 |
| | | S175A, S177T, T180L, R190A, S194V, N235C, | | |
| | | F238W, C277A, E284D, A293G | | |
| 357 | 358 | P5G, D10L, C41A, F49L, W61A, F64T, I79C, | | 1.92 |
| | | K125M, F158G, S175A, S177T, T180R, R190S, | | |
| | | S194A, N235K, F238W, C277M, E284K, A293V | | |
| 359 | 360 | P5G, D10W, C41S, F49L, W61A, F64T, I79A, | | 1.88 |
| | | K125M, F158G, S175A, S177A, T180L, R190Q, | | |
| | | S194L, N235C, F238L, C277M, E284K, A293V | | |
| 361 | 362 | P5G, D10L, C41G, F49R, W61A, F64T, I79C, | | 1.88 |
| | | K125W, F158G, S175T, S177T, T180R, R190S, | | |
| | | S194L, N235K, F238L, C277A, E284D, A293G | | |
| 363 | 364 | P5V, D10L, C41A, F49L, W61A, F64W, I79A, | | 1.86 |
| | | K125V, F158G, S175V, S177T, T180R, R190A, | | |
| | | S194A, N235C, F238L, C277A, E284R, A293G | | |
| 365 | 366 | P5G, D10V, C41A, F49L, W61A, F64M, I79C, | | 1.85 |
| | | K125V, F158G, S175A, S177T, T180L, R190S, | | |
| | | S194V, N235C, F238W, C277M, E284R, A293G | | |
| 367 | 368 | P5G, D10W, C41A, F49L, W61A, F64G, I79C, | | 1.84 |
| | | K125W, F158G, S175T, S177T, T180R, R190S, | | |
| | | S194L, N235C, F238W, C277A, E284K, A293G | | |
| 369 | 370 | P5V, D10L, C41A, F49L, W61V, F64M, I79C, | | 1.81 |
| | | K125V, F158G, S175V, S177T, T180R, R190A, | | |
| | | S194A, N235C, F238W, C277M, E284K, A293V | | |
| 371 | 372 | P5G, D10L, N11D, C41G, F49L, W61V, F64W, | | 1.8 |
| | | I79C, K125M, F158G, S175A, S177T, T180V, | | |
| | | R190A, S194A, N235C, F238L, C277M, E284K, | | |
| | | A293G | | |
| 373 | 374 | P5G, D10V, C41A, F49R, W61A, F64M, I79C, | | 1.79 |
| | | K125W, F158G, S175A, S177A, T180R | | |
| 375 | 376 | P5V, D10V, C41S, F49M, W61A, F64M, I79A, | | 1.79 |
| | | K125W, F158G, S175A, S177G, T180R, R190S, | | |
| | | S194V, N235K, F238L, C277M, E284R, A293G | | |
| 377 | 378 | P5V, D10V, C41A, F49R, W61A, F64L, I79A, | | 1.77 |
| | | K125M, F158G, S175T, S177G, T180V, R190Q, | | |
| | | S194A, N235C, F238L, C277M, E284K, A293V | | |
| 379 | 380 | P5G, D10V, C41A, F49L, W61V, F64G, I79A, | | 1.76 |
| | | K125V, A129T, F158G, S175V, S177A, T180R, | | |
| | | R190Q, S194L, N235V, F238L, C277M, E284D, | | |
| | | A293G | | |
| 381 | 382 | P5G, D10V, C41A, F49L, W61V, F64L, I79A, | | 1.75 |
| | | K125V, F158G, S175A, S177T, T180L, R190A, | | |
| | | S194A, N11D, N235C, F238L, C277M, E284D, | | |
| | | A293G | | |
| 383 | 384 | P5V, D10V, C41A, F49L, W61V, F64T, I79C, | | 1.74 |
| | | K125M, F158G, S175A, S177A, T180L, R190Q, | | |
| | | S194A, N235V, F238W, C277M, E284K, A293G | | |
| 385 | 386 | P5V, D10W, C41S, F49M, W61A, F64L, I79A, | | 1.7 |
| | | K125W, F158G, S175A, S177T, T180V, R190S, | | |
| | | S194A, N235K, F238L, C277A, E284D, A293K | | |
| 387 | 388 | P5V, D10L, C41A, F49L, W61A, F64M, I79C, | V33 | 1.67 |
| | | K125M, F158G, S175A, S177A, T180L, R190S, | (GTT > GTA) | |
| | | S194L, N235C, F238L, C277A, E284R, A293G | | |
| 389 | 390 | P5G, D10V, C41S, F49L, W61V, F64L, I79A, | | 1.67 |
| | | K125W, F158G, S175A, S177G, T180R, R190S, | | |
| | | S194A, N235C, F238L, C277M, E284R, A293G | | |
| 391 | 392 | P5V, D10V, C41A, F49L, W61V, F64M, I79A, | | 1.64 |
| | | K125W, F158G, S175A, S177G, T180V, R190G, | | |
| | | S194A, N235C, F238W, C277A, E284R, A293V | | |
| 393 | 394 | P5V, D10V, C41S, F49M, W61V, F64G, I79C, | | 1.62 |
| | | K125W, F158A, S175T, S177G, T180V, R190S, | | |
| | | S194A, N235K, F238W, C277A, E284D, A293G | | |
| 395 | 396 | P5V, D10W, C41S, F49L, W61A, F64L, I79C, | 1137 | 1.59 |
| | | K125M, F138I, F158G, S175A, S177A, T180L, | (ATC > ATA) | |
| | | R190S, S194V, N235K, F238W, C277M, E284R, | | |
| | | A293G | | |
| 397 | 398 | C41S, F49R, W61A, F64L, I79C, K125V, F158A, | | 1.59 |
| | | S175V, S177A, T180R, R190S, S194L, N235C, | | |
| | | F238W, C277A, E284D, A293G | | |
| 399 | 400 | P5G, D10V, C41A, F49M, W61A, F64G, I79A, | | 1.58 |
| | | K125W, F158G, S175G, S177T, T180R, R190S, | | |
| | | S194V, N235K, F238L, C277A, E284R, A293G | | |
| 401 | 402 | P5G, D10V, C41S, F49L, W61A, F64G, I79A, | | 1.58 |
| | | K125W, F158G, S175A, S177G, T180L, R190Q, | | |
| | | S194V, N235C, F238W, C277M, E284K, A293G | | |
| 403 | 404 | P5G, D10V, C41S, F49M, W61A, F64M, I79A, | | 1.57 |
| | | K125W, F158A, S175A, S177T, T180V, R190S, | | |
| | | S194L, N235C, F238L, C277M, E284D, A293K | | |
| 405 | 406 | P5G, D10V, C41A, F49M, W61A, F64L, I79C, | | 1.54 |
| | | K125M, A129T, F158G, S175G, S177A, T180R, | | |
| | | R190A, S194L, N235K, F238L, C277A, E284D, | | |
| | | A293G | | |
| 407 | 408 | P5G, D10L, C41S, F49M, W61V, F64W, I79C, | | 1.53 |
| | | K125V, F158A, S175T, S177T, T180R, R190S, | | |
| | | S194A, N235K, F238L, C277A, E284R, A293G | | |
| 409 | 410 | P5V, D10W, C41G, F49R, W61V, F64T, I79A, | | 1.51 |
| | | K125V, F158G, S175T, S177A, T180V, R190S, | | |
| | | S194V, N235K, F238L, C277M, E284D, A293G | | |
| 411 | 412 | P5G, D10V, C41A, F49L, W61A, F64M, I79C, | | 1.49 |
| | | K125M, F158G, S175V, S177A, T180V, R190Q, | | |
| | | S194A, N235V, F238W, C277M, E284D, A293G | | |
| 413 | 414 | P5V, D10W, C41S, F49M, W61A, F64T, I79C, | | 1.47 |
| | | K125M, F158G, S175V, S177T, T180V, R190A, | | |
| | | S194A, N235K, F238W, C277A, E284D, A293K | | |
| 415 | 416 | C41A, F49L, W61A, F64T, I79A, K125M, F158A, | | 1.47 |
| | | S175V, S177G, T180R, R190Q, S194A, N235C, | | |
| | | F238W, C277M, E284D, A293G | | |
| 417 | 418 | P5G, D10L, C41S, F49M, W61V, F64M, I79C, | | 1.46 |
| | | K125W, F158A, S175A, S177G, T180R, R190S, | | |
| | | S194A, N235K, F238W, C277M, E284D, A293G | | |
| 419 | 420 | P5V, D10W, C41G, F49R, W61A, F64M, I79A, | | 1.45 |
| | | K125V, F158A, S175V, S177T, T180R, R190S, | | |
| | | S194V, N235C, F238L, C277M, E284R, A293K | | |
| 421 | 422 | P5V, D10V, C41A, F49L, W61V, F64M, I79A, | | 1.43 |
| | | K125W, F158A, S175A, S177G, T180R, R190S, | | |
| | | S194V, N235C, F238L, C277A, E284D, A293G | | |
| 423 | 424 | P5G, D10W, C41G, F49L, W61V, F64W, I79C, | | 1.43 |
| | | K125W, F158G, S175A, S177T, T180V, R190A, | | |
| | | S194A, N235V, F238W, C277M, E284K, A293K | | |
| 425 | 426 | P5G, D10V, C41S, F49M, W61V, F64W, I79A, | | 1.4 |
| | | K125W, F158G, S175V, S177T, T180R, R190Q, | | |
| | | S194A, N235C, F238L, C277A, E284D, A293K | | |
| 427 | 428 | P5G, D10L, C41G, F49L, W61A, F64M, I79A, | | 1.39 |
| | | K125V, F158A, S175A, S177G, T180V, R190G, | | |
| | | S194L, N235V, F238W, C277M, E284R, A286G, | | |
| | | A293G | | |
| 429 | 430 | P5G, D10W, C41A, F49L, W61A, F64M, I79A, | | 1.38 |
| | | K125W, F158G, S175G, S177T, T180L, R190Q, | | |
| | | S194A, N235K, F238L, C277A, E284R, A293G | | |
| 431 | 432 | P5V, D10L, C41S, F49L, W61A, F64L, I79C, | | 1.38 |
| | | K125M, F158A, S175G, S177A, T180R, R190Q, | | |
| | | S194V, N235C, F238L, C277M, E284R, A293G | | |
| 433 | 434 | C41S, F49L, W61A, F64M, I79A, K125M, F158A, | | 1.38 |
| | | S175V, S177T, T180R, R190G, S194L, N235V, | | |
| | | F238L, C277A, E284D, A293G | | |
| 435 | 436 | P5G, D10V, C41A, F49M, W61A, F64T, I79C, | | 1.37 |
| | | K125V, F158A, S175V, S177A, T180L, R190S, | | |
| | | S194A, N235V, F238W, C277M, E284R, A293G | | |
| 437 | 438 | P5G, D10V, C41G, F49R, W61A, F64T, I79A, | | 1.35 |
| | | K125W, F158A, S175G, S177T, T180L, R190S, | | |
| | | S194L, N235C, F238L, C277M, E284D, A293G | | |
| 439 | 440 | P5G, D10W, C41A, F49M, W61V, F64T, I79C, | | 1.35 |
| | | K125M, F158A, S175T, S177A, T180L, R190A, | | |
| | | S194L, N235C, F238W, C277M, E284D, A293G | | |
| 441 | 442 | P5G, D10L, C41A, F49L, W61A, F64L, I79A, | | 1.34 |
| | | K125W, F158A, S175G, S177T, T180V, R190A, | | |
| | | S194A, N235C, F238W, C277M, E284R, A293G | | |
| 443 | 444 | P5G, D10L, C41A, F49M, W61A, F64L, I79C, | | 1.33 |
| | | K125V, F158A, S175G, S177A, T180V, R190A, | | |
| | | F195V, S194L, N235C, F238W, C277A, E284D, | | |
| | | A293G | | |
| 445 | 446 | P5V, D10L, C41G, F49M, W61V, F64T, I79C, | | 1.33 |
| | | K125W, F158A, S175T, S177A, T180V, R190S, | | |
| | | S194V, N235C, F238W, C277A, E284D, A293G | | |
| 447 | 448 | P5G, D10W, C41S, F49L, W61V, F64L, I79A, | F141 | 1.33 |
| | | K125W, F158A, S175A, S177G, T180R, R190G, | (TTC > TTT) | |
| | | S194L, N235C, F238W, C277A, E284R, A293G | | |
| 449 | 450 | P5G, D10V, C41A, F49M, W61A, F64G, I79A, | | 1.33 |
| | | K125V, F158G, S175A, S177A, T180L, R190Q, | | |
| | | S194L, N235C, F238W, C277M, E284D, A293V | | |
| 451 | 452 | P5G, D10W, C41G, F49M, W61A, F64W, I79C, | | 1.3 |
| | | K125M, F158G, S175A, S177A, T180L, R190A, | | |
| | | S194L, N235K, F238W, C277M, E284D, A293G | | |
| 453 | 454 | P5V, D10W, C41G, F49L, W61A, F64W, I79C, | | 1.3 |
| | | K125V, F158G, S175G, S177G, T180L, R190S, | | |
| | | S194A, N235K, F238L, C277A, E284D, A293V | | |
| 455 | 456 | P5G, D10W, C41A, F49L, W61A, F64G, I79C, | | 1.29 |
| | | K125W, F158A, S175A, S177A, T180V, R190S, | | |
| | | S194A, N235K, F238W, C277A, E284R, A293V | | |
| 457 | 458 | P5V, D10L, C41S, F49L, W61A, F64M, I79A, | | 1.29 |
| | | K125W, F158A, S175A, S177A, T180L, R190S, | | |
| | | S194L, N235C, F238L, C277A, E284D, A293G | | |
| 459 | 460 | P5G, D10L, C41A, F49L, W61A, F64M, I79C, | | 1.28 |
| | | K125V, F158A, S175V, S177T, T180R, R190A, | | |
| | | S194A, N235C, F238W, C277M, E284K, A293G | | |
| 461 | 462 | P5G, D10V, C41S, F49L, W61A, F64T, I79C, | | 1.28 |
| | | K125M, F158A, S175T, S177T, T180L, R190Q, | | |
| | | S194L, N235C, F238L, C277A, E284D, A293G | | |
| 463 | 464 | K125W, F158A, S175A, S177T, T180L, R190Q, | | 1.28 |
| | | S194A, N235C, F238L, C277A, E284D, A293V | | |
| 465 | 466 | P5G, D10V, C41A, F49L, W61V, F64L, I79A, | | 1.27 |
| | | K125M, F158A, S175G, S177G, T180L, R190G, | | |
| | | S194V, N235K, F238L, C277A, E284R, A293K | | |
| 467 | 468 | P5V, D10L, C41G, F49L, W61V, F64T, I79C, | | 1.27 |
| | | K125V, F158G, S175G, S177T, T180R, R190S, | | |
| | | S194L, N235C, F238L, C277M, E284R, A293G | | |
| 469 | 470 | P5V, D10V, C41S, F49M, W61A, F64M, I79A, | | 1.26 |
| | | K125W, F158A, S175A, S177G, T180L, R190G, | | |
| | | S194A, N235C, F238L, C277A, E284D, A293V | | |
| 471 | 472 | P5G, D10W, C41S, F49M, W61A, F64W, I79C, | | 1.26 |
| | | K125W, F158A, S175A, S177A, T180R, R190S, | | |
| | | S194A, N235C, F238W, C277M, E284D, A293G | | |
| 473 | 474 | P5V, D10V, C41G, F49L, W61V, F64M, I79C, | | 1.23 |
| | | K125V, F158A, S175G, S177T, T180L, R190Q, | | |
| | | S194A, N235V, F238L, C277M, E284R, A293G | | |
| 475 | 476 | P5V, D10L, C41S, F49L, W61A, F64T, I79C, | | 1.23 |
| | | K125M, F158G, S175G, S177A, T180L, R190A, | | |
| | | S194A, N235C, F238L, C277M, E284K, A293G | | |
| 477 | 478 | P5G, D10V, C41A, F49L, W61A, F64G, I79C, | | 1.23 |
| | | K125W, F158A, S175A, S177T, T180R, R190S, | | |
| | | S194V, N235C, F238W, C277A, E284D, A293G | | |
| 479 | 480 | P5G, D10W, C41G, F49R, W61A, F64L, I79C, | | 1.21 |
| | | K125M, F158A, S175A, S177A, T180L, R190A, | | |
| | | S194V, N235C, F238W, C277M, E284D, A293V | | |
| 481 | 482 | C41A, F49L, W61V, F64L, I79A, K125V, F158G, | | 1.21 |
| | | S175G, S177G, T180L, R190G, S194A, N235V, | | |
| | | F238W, C277A, E284D, A293G | | |
| 483 | 484 | K125W, F158A, S175A, S177G, T180V, R190S, | | 1.19 |
| | | S194L, N235K, F238W, C277M, E284R, A293G | | |
| 485 | 486 | P5V, D10V, C41S, F49M, W61A, F64M, I79C, | | 1.18 |
| | | K125M, F158A, S175G, S177T, T180R, R190S, | | |
| | | S194A, N235V, F238W, C277A, E284R, A293G | | |
| 487 | 488 | P5G, D10W, C41A, F49L, W61A, F64L, I79C, | | 1.17 |
| | | K125W, F158A, S175A, S177G, T180V, R190A, | | |
| | | S194A, N235C, F238W, C277A, E284D, A293G | | |
| 489 | 490 | P5G, D10W, C41S, F49L, W61A, F64W, I79A, | | 1.17 |
| | | K125V, F158A, S175T, S177G, T180V, R190A, | | |
| | | S194L, N235V, F238L, C277M, E284D, A293V | | |
| 491 | 492 | P5V, D10W, C41G, F49L, W61A, F64G, I79A, | | 1.16 |
| | | K125M, F158A, S175G, S177A, T180L, R190S, | | |
| | | S194V, N235V, F238L, C277A, E284D, A293K | | |
| 493 | 494 | P5G, D10L, C41A, F49L, W61A, F64W, I79C, | | 1.16 |
| | | K125W, F158A, S175V, S177A, T180L, R190S, | | |
| | | S194L, N235K, F238W, C277M, E284D, A293G | | |
| 495 | 496 | P5V, D10L, C41G, F49R, W61V, F64M, I79A, | | 1.16 |
| | | K125W, F158A, S175T, S177A, T180L, R190Q, | | |
| | | S194L, N235C, F238W, C277M, E284K, A293V | | |
| 497 | 498 | P5V, D10W, C41G, F49L, W61A, F64G, I79A, | | 1.14 |
| | | K125V, F158A, S175T, S177G, T180V, R190Q, | | |
| | | S194V, N235V, F238W, C277M, E284K, A293G | | |
| 499 | 500 | P5V, D10W, C41G, F49R, W61A, F64L, I79A, | | 1.11 |
| | | K125M, F158G, S175G, S177A, T180L, R190S, | | |
| | | S194L, N235C, F238W, C277M, E284D, A293G | | |
| 501 | 502 | P5V, D10W, C41G, F49R, W61A, F64T, I79A, | | 1.07 |
| | | K125V, F158A, S175A, S177T, T180R, R190G, | | |
| | | S194V, N235K, F238W, C277M, E284D, A293G | | |
| 503 | 504 | P5G, D10W, C41S, F49L, W61A, F64T, I79C, | | 1.05 |
| | | K125V, F158A, S175G, S177A, T180L, R190S, | | |
| | | S194A, N235V, F238L, C277A, E284D, A293G | | |
| 505 | 506 | P5V, D10W, C41G, F49L, W61A, F64M, I79A, | | 1.05 |
| | | K125V, F158A, S175G, S177G, T180L, R190Q, | | |
| | | S194L, N235C, F238L, C277A, E284D, A293V | | |
| 507 | 508 | P5G, D10V, C41S, F49M, W61A, F64W, I79C, | | 1.04 |
| | | K125V, F158G, S175G, S177T, T180R, R190A, | | |
| | | S194L, N235C, F238L, C277M, E284R, A293G | | |
| 509 | 510 | P5G, D10V, C41G, F49R, W61V, F64M, I79A, | | 1.03 |
| | | K125V, F158G, S175G, S177G, T180V, R190S, | | |
| | | S194V, N235K, F238W, C277A, E284K, A293G | | |
| 511 | 512 | P5G, D10L, C41S, F49L, W61V, F64L, I79A, | | 1.02 |
| | | K125M, F158A, S175G, S177G, T180V, R190A, | | |
| | | S194A, N235K, F238W, C277M, E284D, A293G | | |
| 513 | 514 | F49R, W61V, F64M, I79A, K125W, F158A, S175G, | | 1.02 |
| | | S177A, T180V, R190Q, S194V, N235V, F238W, | | |
| | | C277M, E284R, A293G | | |

As shown by the results in Tables 8 and 9, the presence of the following amino acid differences in the recombinant polypeptides having prenyltransferase activity expressed in the strains from the semi-synthetic and fully synthetic libraries resulted in increased CBGA titer produced by the yeast strain: P5G, P5V, H7C, D10L, D10V, D10W, N11D, K34E, C41A, C41G, C41S, R46K, F49L, F49M, F49R, R52P, L54S, W61A, W61V, F64G, F64L, F64M, F64T, F64W, F65L, V68D, I179A, I79C, I79N, I91V, K93N, D95N, I105V, I113N, V115A, I121T, T123K, K125M, K125V, K125W, A129T, F134G, F134V, F138I, I140T, F144S, W153L, F158A, F158G, F158S, I165T, S175A, S175G, S175T, S175V, Y176S, S177A, S177G, S177T, T180I, T180L, T180R, T180V, V188A, V188S, R190A, R190G, R190Q, R190S, F193L, S194A, S194L, S194V, F195V, I196T, I197T, M200R, G204A, G204S, M205G, M205R, S214C, E217G, D219V, N235C, N235K, N235V, F238L, F238W, S241F, V243A, L249A, L249V, S251A, S251C, S253P, W258R, S264Y, M267T, F276L, C277A, C277M, C277R, L278P, F280G, F280L, F280R, Q281R, T282P, E284D, E284K, E284R, A286G, L287F, A288P, Y290S, A291E, A293G, A293K, A293V, S295A, F299L, F301S, I302L, W303C, L304R, L305S, Y307H, Y307S, A308E, A308P, A308R, E309V, Y310C, Y310P, Y310S, F311P, F311S, V312G, Y313H, Y313P, V314A, and F315S.

It also was observed that certain neutral (silent), codon changes, which did not result in an amino acid change in the recombinant polypeptide sequence of Tables 8 and 9, resulted in increased CBGA titer produced by the yeast strain. Specifically, as listed in Tables 8 and 9 the following silent codon changes were observed in the polynucleotide sequences encoding the polypeptides: V33 (GTT>GTC), I37 (ATT>ATC), F73 (TTT>TTC), N74 (AAT>AAC), A78 (GCA>GCG), Q82 (CAA>CAG), K93 (AAG>AAA), P97 (CCA>CCG), V99 (GTT>GTC), S104 (TCA>TCT), L111 (TTA>TTG), L117 (TTG>CTG), G119 (GGT>GGC), F132F (TTC>TTT), V133 (GTT>GTC), G139 (GGT>GGG), R152 (AGA>CGT), Q155 (CAA>CAG), N160 (AAT>AAC), L162 (TTG>CTG), S166 (TCT>TCC), A182 (GCA>GCC), T201 (ACT>ACG), I213 (ATC>ATT), G218 (GGT>GGG), V224 (GTT>GTC), S225 (TCA>TCG), A233 (GCA>GCG), G242 (GGT>GGC), V261 (GTT>GTC), K263 (AAA>AAG), F276 (TTC>TTT), S295 (TCA>TCT), L304 (TTG>CTG), Y306 (TAT>TAC), F311 (TTT>TTC), and V312 (GTT>GTC).

### Example 3: Preparation and Screening of Engineered Polypeptides with Improved Prenyltransferase Activity

This example illustrates preparation of a truncated polypeptide library derived from the parent polypeptide, CsdPT4, of SEQ ID NO: 20 and screening for improved activity in the conversion of OA to CBGA relative to the activity of the parent polypeptide of SEQ ID NO: 20.

Materials and Methods

A. Truncated Polynucleotide Library Build:

The polynucleotide sequence encoding a CsdPT4 polypeptide (SEQ ID NO: 20) from Cannabis sativa was codon optimized as SEQ ID NO: 19 and synthesized as a N-terminal fusion with a gene (SEQ ID NO: 525) encoding the ERG20_{WW} polypeptide (SEQ ID NO: 526). The resulting synthetic gene (SEQ ID NO: 527) encoding the complete ERG20_{WW}-CsdPT4 fusion (SEQ ID NO: 528) was expressed under the Gal1 promoter (SEQ ID NO: 529) and the CYC1 terminator sequence (SEQ ID NO: 540). This synthetic gene was integrated as a knock-in using CRISPR-Cas9 at the NDE1 site in a parent yeast strain, which already had integrated genes encoding the cannabinoid pathway enzyme activities of AAE, OLS, and OAC. The resulting strain, EVP001, integrated with the cannabinoid pathway and the ERG20_{WW}-CsdPT4 gene was used as a control strain in screening the truncation library strains for fold-improvement in CBGA titer as described below. A further screening strain was built by integrating the m-Venus cassette as a N-terminal fusion with the ERG20_{WW}, encoding the ERG20_{WW}-m-Venus polypeptide at the Nde1 site expressed under the Gal1 promoter (SEQ ID NO: 529) and the CYC1 terminator sequence (SEQ ID NO: 530), thereby replacing the previously integrated CsdPT4 gene (SEQ ID NO: 19). This resulting EVP000 strain was no longer capable of converting OA to CBGA.

Genomic DNA from a strain with the ERG20_{WW}-CsdPT4 fusion integrated at NDE1 site (EVP001), was used as the template to generate a library of thirty truncated polynucleotides relative to SEQ ID NO: 20. The truncations were designed to consecutively remove two amino acids at the N-terminal portion of the polypeptide: (1) a first PCR product (Fragment A), amplified 587 base pairs upstream of CsdPT4; (2) a second PCR product (Fragment B), amplified the CsdPT4 coding sequence while consecutively removing six base pairs relative to the N-terminus position, together with 270 base pairs downstream of CsdPT4 (CYC terminator). Fragment B was amplified with a series of 30 forward primer sequences of SEQ ID NO: 726-755 that consecutively removed six nucleotides at the N-terminal position of CsdPT4 and a single reverse primer of SEQ ID NO: 756. Fragment A was amplified using a single forward primer of SEQ ID NO: 757, and a single reverse primer of SEQ ID NO: 758. The two fragments A and B were assembled by overlap extension PCR using a forward primer of SEQ ID NO: 759 and reverse primer of SEQ ID NO: 760. The assembled PCR products were then pooled together, and gel purified to provide a truncated polynucleotide library in the form of linear donor DNA.

The pooled truncated polynucleotide library in the form of linear donor DNA was transformed in a yeast strain (EVP000), which, like EVP001, already had integrated genes encoding the cannabinoid pathway enzyme activities of AAE, OLS, and OAC. The library of linear donor DNA was integrated into EVP000 as a knock-in using CRISPR-Cas9 to replace an m-Venus cassette having an ORF of SEQ ID NO: 531 located at the NDE1 site under control the Gal1 promoter and CYC1 terminator.

B. Screening of the Polynucleotide Truncated Library for Cannabinoid Biosynthesis:

Individual clones from the polynucleotide truncated library integrated into EVP000 and the EVP001 control strain were picked and grown in 0.3 mL YPD in 96-well plates. The culture plates were incubated in shaking incubators for 48 h at 30 C, 85% humidity, and 250 rpm. Cultures were then sub-cultured into 0.27 mL fresh YPD and fed with hexanoic acid (HA) to 2 mM final concentration. Subculture plates were grown in shaking incubators for 48 hours at 30 C, 85% humidity, and 250 rpm. The whole broth from these sub-culture plates was extracted and analyzed for the presence of the cannabinoid precursor compound, OA, and the cannabinoid product, CBGA, using HPLC, as described below.

1. HPLC sample preparation: The whole broth of the culture was extracted and diluted with MeOH for sample preparation. The prepared samples were loaded onto RapidFire365 coupled with a triple quadruple mass spectrometry detector. Metabolites OA and CBGA were detected using MRM mode. Calibration curves of OA and CBGA were generated by running serial dilutions of standards, and then used to calculate concentrations of each metabolite.

2. HPLC instrumentation and parameters: HPLC system: Agilent RapidFire 365; Column: Agilent Cartridge C18 (12 μl, type C); Mobile phase: Pump 1 uses 95:5 H2O:acetonitrile with 0.1% formic acid at 1 mL/min; Pump 2 uses 20:80 acetonitrile:H₂O at 0.8 mL/min; Pump 3 uses MeOH with 0.1% formic acid at 0.8 mL/min; Aqueous wash uses H₂O; Organic wash uses acetonitrile; RapidFire cycle time: Aspiration 600 ms; Load/wash 3000 ms; Extra wash 2000 ms; Elute 4000 ms; Re-equilibration 500 ms.

C. Sequencing

Those clones from the polynucleotide truncated library determined by screening to exhibit an increased CBGA titer were re-tested and sequenced using Sanger sequencing technology to determine the specific truncation differences.

D. Results

Screening of the polynucleotide truncated library strains for fold-improvement in production of CBGA titer from HA feeding (FIOPC), relative to the control strain, EVP001, which expresses the parent CsdPT4 polypeptide of SEQ ID NO: 20, are summarized in Table 10 (below).

TABLE 10

| NT SEQ | AA SEQ | AA | NT | |
|---|---|---|---|---|
| ID NO: | ID NO: | difference | difference | FIOPC |
| 515 | 516 | −12 | −36 | 1.343 |
| 517 | 518 | −10 | −30 | 1.365 |
| 519 | 520 | −8 | −24 | 1.457 |
| 521 | 522 | −4 | −12 | 1.342 |
| 523 | 524 | −2 | −6 | 1.306 |

### Example 4: Preparation and Screening of Engineered Polypeptides with Improved Prenyltransferase Activity

This example illustrates preparation of strains where the synthetic gene (SEQ ID NO: 527) encoding the complete ERG20_{WW}-CsdPT4 fusion (SEQ ID NO: 528) was expressed under the Gal1 promoter (SEQ ID NO: 529) and the CYC1 terminator sequence (SEQ ID NO: 530) at various loci.

Materials and Methods

A. Donor Builds for Integration of ERG20_{WW}-CsdPT4 Fusion (SEQ ID NO: 528) at Various Loci:

The polynucleotide sequence encoding a CsdPT4 polypeptide (SEQ ID NO: 20) from Cannabis sativa was codon optimized as SEQ ID NO: 19 and synthesized as a N-terminal fusion with a gene (SEQ ID NO: 525) encoding the ERG20_{WW} polypeptide (SEQ ID NO: 526). The resulting synthetic gene (SEQ ID NO: 527) encoding the complete ERG20_{WW}-CsdPT4 fusion (SEQ ID NO: 528) was expressed under the Gal1 promoter (SEQ ID NO: 529) and the CYC1 terminator sequence (SEQ ID NO: 530). This synthetic gene was integrated as a knock-in using CRISPR-Cas9 at various sites in a parent yeast strain, which did not have any other integrated cannabinoid pathway genes. Therefore, the resulting strains were fed with olivetolic acid substrate (OA), to screen the strains for relative CBGA titer.

Homology arms were added to the 5′ and 3′ ends of the synthetic gene (SEQ ID NO: 527) encoding the complete ERG20_{WW}-CsdPT4 fusion (SEQ ID NO: 528) was expressed under the Gal1 promoter (SEQ ID NO: 529) and the CYC1 terminator sequence (SEQ ID NO: 530) via PCR. The following loci were investigated for optimal expression of the complete ERG20_{WW}-CsdPT4 fusion (SEQ ID NO: 528) as single integrations or in some cases, as double integrations; ΔNDE1, XII-5 & ΔNDE1, ΔROQ1& ΔNDE1, XII-5, ΔGal80, ΔROQ1. In some examples the integration of the complete ERG20_{WW}-CsdPT4 fusion (SEQ ID NO: 528) resulted in a knockout of the native gene at that locus (NDE1, ROQ1, Gal80).

The individual linear DNA donors with various 5′ and 3′ homology sequences were transformed in a yeast strain which contained a copy of truncated HMG1 gene integrated at the XII-2 locus and a copy of the mutant ERG20_{WW} gene (SEQ ID NO: 526) integrated at the gal80 locus, resulting in a gal80 knockout (MV021). This base screening strain, MV021, was used as the control to determine level of production of CBGA titer from OA feeding (FIOPC).

B. Screening of the ERG20_{WW}-CsdPT4 fusion (SEQ ID NO: 528) at various loci for evaluation of cannabinoid biosynthesis:

Individual clones from the linear DNA donors integrated at various loci were picked and grown in 0.3 mL YPD in 96-well plates along with the control MV021. The culture plates were incubated in shaking incubators for 48 h at 30 C, 85% humidity, and 250 rpm. Cultures were then sub-cultured into 0.27 mL fresh YPD and fed with hexanoic acid (OA) to 3 mM final concentration. Subculture plates were grown in shaking incubators for 48 hours at 30 C, 85% humidity, and 250 rpm. The whole broth from these sub-culture plates was extracted and analyzed for the presence of the cannabinoid precursor compound, OA, and the cannabinoid product, CBGA, using HPLC, as described below.

1. HPLC sample preparation: The whole broth of the culture was extracted and diluted with MeOH for sample preparation. The prepared samples were loaded onto RapidFire365 coupled with a triple quadruple mass spectrometry detector. Metabolites OA and CBGA were detected using MRM mode. Calibration curves of OA and CBGA were generated by running serial dilutions of standards, and then used to calculate concentrations of each metabolite.

2. HPLC instrumentation and parameters: HPLC system: Agilent RapidFire 365; Column: Agilent Cartridge C18 (12 μl, type C); Mobile phase: Pump 1 uses 95:5 H2O:acetonitrile with 0.1% formic acid at 1 mL/min; Pump 2 uses 20:80 acetonitrile:H₂O at 0.8 mL/min; Pump 3 uses MeOH with 0.1% formic acid at 0.8 mL/min; Aqueous wash uses H₂O; Organic wash uses acetonitrile; RapidFire cycle time: Aspiration 600 ms; Load/wash 3000 ms; Extra wash 2000 ms; Elute 4000 ms; Re-equilibration 500 ms.

C. Sequencing

Those clones from the various loci integration builds determined by screening to exhibit a CBGA titer higher than the control, were re-tested and sequenced using Sanger sequencing technology to confirm presence of the ERG20_{WW}-CsdPT4 fusion (SEQ ID NO: 528) at the correct loci.

D. Results

Screening of the various strains with ERG20_{WW}-CsdPT4 fusion (SEQ ID NO: 528) integrated at various loci, for evaluation of level of production of CBGA titer from OA feeding (FIOPC), relative to the control strain, MV021, which does not express a prenyl transferase gene are summarized in Table 11 (below).

TABLE 11

| | Genome locus for | Mean CBGA | Std dev. CBGA |
|---|---|---|---|
| Strain | ERG20ww:CsdPT4 | titer (mg/L) | titer |
| MV326 | ΔNDE1 | 875.6 | 82 |
| MV327 | XII-5, ΔNDE1 | 807.5 | 20 |
| MV023 | ΔROQ1, ΔNDE1 | 288.0 | 41 |
| MV020 | XII-5 | 254.5 | 37 |
| MV331 | ΔGal80 | 264.7 | 46 |
| MV022 | ΔROQ1 | 48.3 | 1 |
| MV021 | No PT | 0.0 | 0 |

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. A recombinant polypeptide having prenyltransferase activity, wherein the polypeptide comprises an amino acid sequence of at least 80% identity to SEQ ID NO: 20 and an amino acid difference as compared to SEQ ID NO: 20 at one or more positions selected from: W61, F64, I79, F134, W153, F158, S175, S177, T180, N235, E284, and A293; optionally, wherein the amino acid differences are selected from: W61A, W61V, F64G, F64L, F64M, F64T, F64W, I179A, I79C, I79N, I79S, F134G, F134V, W153L, F158A, F158G, F158S, S175A, S175G, S175T, S175V, Y176S, S177A, S177G, S177T, T180I, T180L, T180R, T180V, N235C, N235K, N235V, E284D, E284K, E284R, A293G, A293K, and A293V.

2. The polypeptide of claim 1, wherein the polypeptide further comprises an amino acid residue difference as compared to SEQ ID NO: 20 at one or more positions selected from: P5, H7, D10, N11, K34, C41, R46, F49, N50, R52, L54, G58, F65, V68, F75, M80, D87, I91, K93, D95, V99, I105, E106, I113, V115, I121, T123, K125, A129, F138, I140, F144, F161, I165, F173, Y176, S181, V188, R190, F193, S194, F195, I196, I197, M200, G204, M205, S214, E217, D219, T229, F238, S241, V243, L249, S251, S253, W258, S264, M267, F276, C277, L278, F280, Q281, T282, A286, L287, A288, Y290, A291, P294, S295, F299, F301, I302, W303, L304, L305, Y307, A308, E309, Y310, F311, V312, Y313, V314, and F315; optionally, wherein the amino acid differences are selected from: P5G, P5V, H7C, D10L, D10V, D10W, N11D, K34E, C41A, C41G, C41S, R46K, F49L, F49M, F49R, N50D, R52P, L54S, G58S, F65L, V68D, F75W, M80V, D87E, I91V, K93N, D95N, V99A, I105V, E106R, I113N, I113W, V115A, I121T, T123K, K125M, K125V, K125W, A129T, F138I, I140T, F144S, F161V, I165L, I165T, F173I, Y176S, S181R, V188A, V188S, R190A, R190G, R190Q, R190S, F193L, S194A, S194L, S194V, F195V, I196T, I197T, M200R, G204A, G204S, M205G, M205R, S214C, E217G, D219V, T229V, F238L, F238W, S241F, V243A, L249A, L249V, S251A, S251C, S253P, W258R, S264Y, M267T, F276L, C277A, C277M, C277R, L278P, F280G, F280L, F280R, Q281R, T282P, A286G, L287F, A288P, Y290S, A291E, P294E, S295A, F299L, F301S, I302L, W303C, L304R, L305S, Y307H, Y307S, A308E, A308P, A308R, E309V, Y310C, Y310P, Y310S, F311P, F311S, V312G, Y313H, Y313P, V314A, and F315S.

3. The polypeptide of claim 1, wherein the polypeptide comprises a combination of amino acid differences selected from:
F64T, E284R
F64T, I79C
F64T, S177A
F64T, T180R
F64T, I79C, W153L
F64T, I79C, F134G
F64T, F134G, S177A
F64T, S175V, S177A
F64T, I79C, W153L, T180R
F64T, F134G, S175V, S177A
F64T, F134G, S177A, T180R
F64T, F134G, W153L, S177T, T180R
F64T, I79A, W153L, S175V, T180R
F64T, I79A, W153L, S177A, T180R
F64T, F134G, S175T, S177T, T180R
F64T, I79C, F134G, S177A
F64T, I79C, F134G, S177A, T180R
F64T, F134G, W153L, S177A, T180R, E284K
F64T, I79C, W153L, S175T, S177T, T180R
F64T, I79C, W153L, S175T, T180R
F64T, W153L, S175V, S177A
F64T, W153L, S175V, S177A, T180R
F64T, W153L, S175V, S177T
F64T, W153L, S175V, V188A, R190S
I79A, S177T
I79C, F134G
I79C, W153L
I79A, F134G, S175V
I79A, F134G, W153L
I79A, S175V, S177T
I79A, S177A, T180R
I79A, W153L, E284K
I79C, S175V, S177T
I79C, S177A, T180R
I79C, W153L, S175T
I79C, W153L, S177T
I79A, S175V, T180R
I79A, W153L, S175T, T180R
I79C, F134G, W153L, S175V
I79C, S175T, S177T, T180R
I79C, F134G, S177A, T180R
I79C, W153L, S177T, T180R
I79A, W153L, S175V, S177T, T180R
I79C, F134G, S175T, S177A, T180R
I79C, F134G, W153L, S175T, T180I
I79C, W153L, S175V, S177T, T180R
I79N, F134G, S175V, S177T, T180R
I79A, F134G, W153L, S175T, S177T, T180R
F134G, S177T
F134G, T180R
F134G, W153L
F134G, W153L, S175V
F134G, W153L, S177T
F134G, S177T, E284R
F134G, S175T, T180R
F134G, W153L, S175T, S177A
F134G, W153L, S175T, S177T
F134G, W153L, S175T, T180R
F134G, W153L, S177T, T180R
F134G, S175T, S177T, T180R
F134G, S175V, S177A, T180R
F134G, W153L, S175V, S177T, T180R
W153L, S175T
W153L, T180R
W153L, S175V
W153L, S175T, S177T
W153L, S177A, T180R
S175V, S177T, T180R
W153L, S175T, S177A, T180R
W153L, S175V, S177A, T180R
W153L, S175V, S177T, T180R
S175V, S177T, T180R, E284K
S177A, T180R
S177T, T180R
W61A, F64G, I79A, F158A, S175G, S177A, T180L, N235V, E284D, A293K
W61A, F64G, I79A, F158A, S175T, S177G, T180V, N235V, E284K, A293G
W61A, F64G, I79A, F158G, S175A, S177A, T180L, N235C, E284D, A293V
W61A, F64G, I79A, F158G, S175A, S177G, T180L, N235C, E284K, A293G
W61A, F64G, I79A, F158G, S175G, S177T, T180R, N235K, E284R, A293G
W61A, F64G, I79C, F158A, S175A, S177A, T180V, N235K, E284R, A293V
W61A, F64G, I79C, F158A, S175A, S177T, T180R, N235C, E284D, A293G
W61A, F64G, I79C, F158G, S175A, S177T, T180V, N235K, E284D, A293G
W61A, F64G, I79C, F158G, S175T, S177T, T180R, N235C, E284K, A293G
W61A, F64L, I79A, F158A, S175G, S177T, T180V, N235C, E284R, A293G
W61A, F64L, I79A, F158G, S175A, S177T, T180V, N235K, E284D, A293K
W61A, F64L, I79A, F158G, S175G, S177A, T180L, N235C, E284D, A293G
W61A, F64L, I79A, F158G, S175T, S177G, T180V, N235C, E284K, A293V
W61A, F64L, I79C, F158A, S175A, S177A, T180L, N235C, E284D, A293V
W61A, F64L, I79C, F158A, S175A, S177G, T180V, N235C, E284D, A293G
W61A, F64L, I79C, F158A, S175G, S177A, T180R, N235C, E284R, A293G
W61A, F64L, I79C, F158A, S175G, S177A, T180V, N235C, E284D, A293G
W61A, F64L, I79C, F158A, S175V, S177A, T180R, N235C, E284D, A293G
W61A, F64L, I79C, F158G, S175A, S177A, T180L, N235K, E284R, A293G
W61A, F64L, I79C, F158G, S175G, S177A, T180R, N235K, E284D, A293G
W61A, F64L, I79C, F158G, S175V, S177A, T180V, N235C, E284K, A293K
W61A, F64M, I79A, F158A, S175A, S177A, T180L, N235C, E284D, A293G
W61A, F64M, I79A, F158A, S175A, S177G, T180L, N235C, E284D, A293V
W61A, F64M, I79A, F158A, S175A, S177G, T180V, N235V, E284R, A293G
W61A, F64M, I79A, F158A, S175A, S177T, T180V, N235C, E284D, A293K
W61A, F64M, I79A, F158A, S175G, S177G, T180L, N235C, E284D, A293V
W61A, F64M, I79A, F158A, S175V, S177T, T180R, N235C, E284R, A293K
W61A, F64M, I79A, F158A, S175V, S177T, T180R, N235V, E284D, A293G
W61A, F64M, I79A, F158G, S175A, S177G, T180R, N235K, E284R, A293G
W61A, F64M, I79A, F158G, S175G, S177T, T180L, N235K, E284R, A293G
W61A, F64M, I79C, F158A, S175G, S177T, T180R, N235V, E284R, A293G
W61A, F64M, I79C, F158A, S175V, S177T, T180R, N235C, E284K, A293G
W61A, F64M, I79C, F158G, S175A, S177A, T180L, N235C, E284R, A293G
W61A, F64M, I79C, F158G, S175A, S177A, T180R
W61A, F64M, I79C, F158G, S175A, S177T, T180L, N235C, E284R, A293G
W61A, F64M, I79C, F158G, S175V, S177A, T180V, N235V, E284D, A293G
W61A, F64T, I79A, F158A, S175A, S177T, T180R, N235K, E284D, A293G
W61A, F64T, I79A, F158A, S175G, S177T, T180L, N235C, E284D, A293G
W61A, F64T, I79A, F158A, S175V, S177G, T180R, N235C, E284D, A293G
W61A, F64T, I79A, F158G, S175A, S177A, T180L, N235C, E284K, A293V
W61A, F64T, I79C, F158A, S175G, S177A, T180L, N235V, E284D, A293G
W61A, F64T, I79C, F158A, S175T, S177T, T180L, N235C, E284D, A293G
W61A, F64T, I79C, F158A, S175V, S177A, T180L, N235V, E284R, A293G
W61A, F64T, I79C, F158G, S175A, S177T, T180R, N235K, E284K, A293V
W61A, F64T, I79C, F158G, S175G, S177A, T180L, N235C, E284K, A293G
W61A, F64T, I79C, F158G, S175T, S177T, T180R, N235K, E284D, A293G
W61A, F64T, I79C, F158G, S175V, S177T, T180V, N235K, E284D, A293K
W61A, F64W, I79A, F158A, S175T, S177G, T180V, N235V, E284D, A293V
W61A, F64W, I79A, F158G, S175A, S177A, T180L, N235K, E284D, A293V
W61A, F64W, I79A, F158G, S175V, S177T, T180R, N235C, E284R, A293G
W61A, F64W, I79C, , F158G, S175G, S177G, T180L, N235K, E284D, A293V
W61A, F64W, I79C, F158A, S175A, S177A, T180R, N235C, E284D, A293G
W61A, F64W, I79C, F158A, S175V, S177A, T180L, N235K, E284D, A293G
W61A, F64W, I79C, F158G, S175A, S177A, T180L, N235K, E284D, A293G
W61A, F64W, I79C, F158G, S175G, S177T, T180R, N235C, E284R, A293G
W61V, F64G, I79A, F158G, S175V, S177A, T180R, N235V, E284D, A293G
W61V, F64G, I79C, F158A, S175T, S177G, T180V, N235K, E284D, A293G
W61V, F64L, I79A, F158A, S175A, S177G, T180R, N235C, E284R, A293G
W61V, F64L, I79A, F158A, S175G, S177G, T180L, N235K, E284R, A293K
W61V, F64L, I79A, F158A, S175G, S177G, T180V, N235K, E284D, A293G
W61V, F64L, I79A, F158G, S175A, S177G, T180R, N235C, E284R, A293G
W61V, F64L, I79A, F158G, S175A, S177T, T180L, N235C, E284D, A293G
W61V, F64L, I79A, F158G, S175G, S177G, T180L, N235V, E284D, A293G
W61V, F64M, I79A, F158A, S175A, S177G, T180R, N235C, E284D, A293G
W61V, F64M, I79A, F158A, S175G, S177A, T180V, N235V, E284R, A293G
W61V, F64M, I79A, F158A, S175T, S177A, T180L, N235C, E284K, A293V
W61V, F64M, I79A, F158G, S175A, S177G, T180V, N235C, E284R, A293V
W61V, F64M, I79A, F158G, S175G, S177G, T180V, N235K, E284K, A293G
W61V, F64M, I79C, F158A, S175A, S177G, T180R, N235K, E284D, A293G
W61V, F64M, I79C, F158A, S175G, S177T, T180L, N235V, E284R, A293G
W61V, F64M, I79C, F158G, S175V, S177T, T180R, N235C, E284K, A293V
W61V, F64T, I79A, F158G, S175A, S177T, T180L, N235C, E284D, A293G
W61V, F64T, I79A, F158G, S175T, S177A, T180V, N235K, E284D, A293G
W61V, F64T, I79C, F158A, S175T, S177A, T180L, N235C, E284D, A293G
W61V, F64T, I79C, F158A, S175T, S177A, T180V, N235C, E284D, A293G
W61V, F64T, I79C, F158G, S175A, S177A, T180L, N235V, E284K, A293G
W61V, F64T, I79C, F158G, S175G, S177T, T180R, N235C, E284R, A293G
W61V, F64W, I79A, F158G, S175V, S177T, T180R, N235C, E284D, A293K
W61V, F64W, I79C, F158G, S175A, S177T, T180V, N235C, E284K, A293G
W61V, F64W, I79C, F158G, S175A, S177T, T180V, N235V, E284K, A293K
W61V, F64W, I79C, F158A, S175T, S177T, T180R, N235K, E284R, A293G
F158A, S175A, S177G, T180V, N235K, E284R, A293G
F158A, S175A, S177T, T180L, N235C, E284D, A293V

4. The polypeptide of claim 1, wherein the polypeptide comprises a combination of amino acid differences selected from:
P5G, H7C, C41S, F64T, F134G, S175V, S177A, G204S, L249A, S295A
H7C, D10V, C41A, R46K, F64T, I79C, K125W, F134G
H7C, D10V, I79C, F134G, S175T, S177A, T180R, R190S, G204S
P5G, H7C, D10V, T180R, G204S, S241F
D10V, C41S, F64T, I79C, W153L, S175T, T180R, G204S, L249A, C277M, F280R,
Q281R, A291E, S295A, Y307H, A308E, E309V, Y310S
P5G, H7C, C41A, F64T, I79A, I113N, W153L, S175V, T180R, S194L, I197T, G204S
P5G, H7C, D10V, F64T, W153L, S175V, V188A, R190S
H7C, R46K, I79C, K125W, F134G, Y176S, S177A, T180R, G204S, C277A, L278P,
F280G, Q281R, T282P
H7C, C41S, R46K, I79C, K125W, S175T, S177T, T180R, R190S, G204S, S251A,
C277M, Q281R, A291E
P5G, C41A, K125W, W153L, S175T, S177A, T180R
H7C, S177T, T180R, S194V, G204A, S295A
P5G, H7C, C41A, F64T, K125W, F134G, S177A, G204S, C277A, F280R, F301S
D10V, C41S, R46K, F134G, W153L, S177T, T180R, V188A, R190S, M205G, L249A,
C277M, F280R
P5G, H7C, D10V, F49L, R52P, K125W, W153L, S175V, S177T, T180R, S194L, G204S,
M205G
H7C, D10V, C41A, R46K, R52P, S175V, S177A, T180R, V188A, G204S, M205G
P5G, H7C, I79C, F134G, W153L, S175V
D10V, C41S, R46K, F134G, W153L, S175V, G204S
H7C, R46K, I79A, S177A, T180R, V188A, G204S
H7C, D10V, C41A, K125W, W153L, S175T, V188S, R190S, M200R, M205G, S214C,
D219V, V243A, S251C, S264Y, Q281R, A288P
H7C, K125W, S175V, S177T, T180R, S194L, G204S, S251A, S295A
H7C, D10V, C41A, R46K, V68D, I79A, W153L, S175V, S177T, T180R, V188A, R190S,
G204S, M205G
P5G, R46K, R52P, F64T, L249A, E284R, A291E, S295A
P5G, H7C, C41S, R46K, K125W, F134G, I165T, S175T, S177T, T180R, G204S, Q281R,
S295A
H7C, D10V, C41A, F64T, W153L, S175V, S177A, T180R, M205G
P5G, C41S, R52P, I79C
P5G, H7C, R52P, I79A, F134G, W153L, G204S, M205G, L249A, C277A, F280R,
Q281R, A291E
D10V, R46K, W153L, T180R, S194V, L249A, C277M, F280R, Q281R, A291E, S295A
D10V, C41S, K125W, F134G, S175V, S177A, T180R
P5G, D10V, C41S, K125W, F134G, T180R
H7C, I79A, K125W, W153L, S175T, T180R, R190S, M205G, L249A, S251A, C277A,
F280R, A291E
P5G, H7C, D10V, C41S, R46K, I121T, K125W, F134G, W153L, S175V, S177T, T180R,
V188A, R190S, M205G
P5G, C41S, S175V, S177A, T180R, L249A, C277A
H7C, D10V, F49L, I79C, W153L, S175V, S177T, T180R, V188A, S194L
P5G, H7C, C41S, F49L, R52P, F64T, I79C, K125W, W153L, S175T, S177T, T180R,
V188A, R190S, G204S, C277M, A291E
P5G, H7C, D10V, F64T, F134G, W153L, S177T, T180R, L249A, C277A, Q281R,
A291E, S295A
H7C, D10V, I79C, S177A, T180R, V188A, R190S, A291E, S295A
H7C, D10V, C41S, R46K, I79A, K125W, S175V, T180R, R190S
P5G, H7C, D10V, I79C, K125W, S175V, S177T, R190S, G204S, M205G, L249A,
S251A, C277M, A291E
D10V, R46K, K125W, W153L, S175V, S177A, T180R, V188A, C277M, S295A
P5G, H7C, C41S, F64T, K125W, F134G, S175V, S177A, V188A, M205G
P5G, C41A, R46K, F134G, S175T, T180R, V188A, R190S
H7C, D10V, C41A, R52P, K125W, F134G, S177T, S194V
P5G, D10V, R52P, I79C, K125W, W153L
P5G, H7C, D10V, F49L, K125W, F134G, W153L, S194L, G204S, Q281R, S295A
P5G, R52P, F64T, I79C, D95N
P5G, H7C, I79A, I105V, S177A, T180R, S194V, M205G, Q281R, S295A, V314A
D10V, C41S, R46K, R52P, F64T, F134G, S177A, T180R
P5G, D10V, R46K, R52P, F64T, I79C, K125W, W153L, T180R
H7C, C41S, R52P, I79C, K125W, F134G, W153L, S175T, T180I, V188A, R190S, L249A,
S251A, F280R, Q281R, S295A
H7C, D10V, K125W, F134G, W153L, S175T, S177T
P5G, C41S, R46K, K125W, F134G, W153L, S175T, S177A, S194V
P5G, D10V, I79A, K125W, F134G, S175V, C277M, Q281R, A291E, S295A
P5G, D10V, C41A, R46K, R52P, F134G, W153L, S175T, S177T, M205G, L249A,
S251A, F280R, Q281R, A291E
P5G, C41S, I79C, W153L, S175T, M205G, L249A, S251A, C277A, F280R, A291E,
Y313P
P5G, H7C, D10V, C41A, R52P, T123K, K125W
P5G, D10V, C41A, R46K, K125W, F134G, W153L, S175V, S177T, T180R, G204S,
M205G, S253P, F280R, A291E, S295A, F315S
H7C, C41A, F49L, I79C, K125W, W153L, S177T
P5G, H7C, C41A, S177A, T180R, M205G, L249A, S251A, C277A, A291E, S295A,
F299L
H7C, D10V, C41S, F64T, S177A, V188A, M205G, L249A, S251A, A291E, S295A
H7C, D10V, R46K, F134V
P5G, H7C, F49L, F64T, I79C, K125W, W153L, S175T, S194L, C277M, F280R, Q281R,
A291E, S295A
P5G, D10V, F64T, F134G, S175T, S177T, T180R, L249A, S251A, A291E
P5G, H7C, D10V, I79A, K125W, W153L, E284K, A291E
P5G, D10V, F49L, R52P, F134G, S194L, S251A, W258R, Q281R, A291E, S295A
H7C, D10V, C41S, I79C, F134G, V188A, R190S, S214C, A291E, S295A, F311S
C41A, I79A, K125W, F134G, W153L, S175T, S177T, T180R
P5G, H7C, I79A, S175V, S177T, L249A, S251A, W258R, Q281R
H7C, D10V, C41A, R46K, F64T, V115A, K125W, T180R, C277A, F280R, S295A
D10V, C41A, R46K, R52P, F64T, K125W, F134G, W153L, S177A, T180R, S214C,
L249A, E284K, S295A, A308P
P5G, D10V, C41A, R46K, I79A, F134G, W153L, L249A, Q281R, S295A
P5G, H7C, R46K, K125W, S175V, S177T, T180R, M205G, L249A, E284K, S295A
D10V, C41S, R46K, F65L, K125W, W153L, S177A, T180R, S194L, L249A, S251A,
C277A
C41A, R46K, I79C, K93N, K125W
P5G, H7C, R52P, I79C, K125W, W153L, S177T, T180R, S194L
C41A, R46K, W153L, S175V, M205G
H7C, R52P, W153L, S175V, G204S, M205G, C277M, Q281R, S295A
P5G, R52P, I79A, K125W
P5G, H7C, D10V, C41S, F49L, R52P, F64T, S175V, S177A, M205G
P5G, F49L, I79A, S177T
P5G, D10V, C41S, R46K, I79N, F134G, S175V, S177T, T180R, M205G, A291E, S295A
H7C, D10V, R46K, K125W, F158S, S175V, S177T, T180R, V188A, R190S, M205R,
L249A, C277R, F280R, Q281R, A291E, Y310S
P5G, H7C, C41A, R52P, F134G, W153L, S175T, L249A, S251A, E284R, S295A, L305S
H7C, C41S, R46K, K125W, S194L, Q281R, A291E
H7C, F49L, K125W, F134G, S177T, M205G, E284R, A291E, S295A
P5G, H7C, D10V, C41A, R46K, F134G, F144S, W153L, G204S, L249A, S251A, C277M,
F280R, Q281R
P5G, R46K, L54S, K125W, W153L, S175T, S177T, S214C, F276L, F280R, Q281R,
A308P, Y310C, Y313H
P5G, D10V, R46K, I79C
P5G, D10V, R46K, W153L, S175V, S177T, T180R, S214C, S251A, C277M, F280R,
Q281R, S295A, F301S, I302L, W303C, L304R, Y310S, F311S
D10V, R46K, F64T, I79A, W153L, S177A, T180R, S194L, S251A, S295A
H7C, D10V, C41A, S175V, F193L
H7C, C41A, R46K, R52P, K125W, F134G, S177T
P5G, H7C, D10V, C41A, K125W, S194L, G204S, M205G, F280R, A291E, S295A
P5G, F49L, R52P, K125W, F134G, W153L, S177T, R190S, M205G, S214C, F280R,
A291E, S295A, V312G, Y313H
H7C, D10V, K125W, F134G
D10V, F49L, R52P, F64T, W153L, S175V, S177A, Q281R, S295A, F311P
H7C, D10V, C41S, R46K, K125W, W153L, S194A
R52P, F64T, I79C, F134G, S177A, T180R, L249A, M267T, C277M, Q281R, L287F,
A288P, Y290S
D10V, F64T, I79C, K125W, F134G, I140T, S177A, L249A, C277M, Q281R, A291E
D10V, K34E, F49L
H7C, D10V, C41A, R46K, F64T, K125W, R190S, M205G, C277A, S295A, Y307S,
A308R, Y310S
P5G, H7C, D10V, C41G, K125W, F134G, L249V, F280L, A291E
P5G, F64T, I79C, W153L, I165T, Q281R, A291E, S295A
H7C, R46K, F64T, I91V, W153L, S175V, S177T, I196T, M205G, L249A, C277M, A291E,
Y310P
H7C, C41A, K125W, F134G, W153L, S175T, T180R, R190S, M205G, E217G
P5G, D10W, C41G, F49M, W61A, F64W, I79A, K125M, F158G, S175A, S177A, T180L,
R190S, S194V, N235K, F238W, C277A, E284D, A293V
P5G, D10V, C41S, F49M, W61A, F64L, I79C, K125V, F158G, S175V, S177A, T180V,
R190S, S194V, N235C, F238L, C277A, E284K, A293K
P5G, D10V, C41A, F49L, W61A, F64G, I79C, K125V, F158G, S175A, S177T, T180V,
R190S, S194V, N235K, F238W, C277A, E284D, A293G
C41A, F49L, W61V, F64T, I79A, K125M, F158G, S175A, S177T, T180L, R190A, S194V,
N235C, F238W, C277A, E284D, A293G
P5G, D10L, C41A, F49L, W61A, F64T, I79C, K125M, F158G, S175A, S177T, T180R,
R190S, S194A, N235K, F238W, C277M, E284K, A293V
P5G, D10W, C41S, F49L, W61A, F64T, I79A, K125M, F158G, S175A, S177A, T180L,
R190Q, S194L, N235C, F238L, C277M, E284K, A293V
P5G, D10L, C41G, F49R, W61A, F64T, I79C, K125W, F158G, S175T, S177T, T180R,
R190S, S194L, N235K, F238L, C277A, E284D, A293G
P5V, D10L, C41A, F49L, W61A, F64W, I79A, K125V, F158G, S175V, S177T, T180R,
R190A, S194A, N235C, F238L, C277A, E284R, A293G
P5G, D10V, C41A, F49L, W61A, F64M, I79C, K125V, F158G, S175A, S177T, T180L,
R190S, S194V, N235C, F238W, C277M, E284R, A293G
P5G, D10W, C41A, F49L, W61A, F64G, I79C, K125W, F158G, S175T, S177T, T180R,
R190S, S194L, N235C, F238W, C277A, E284K, A293G
P5V, D10L, C41A, F49L, W61V, F64M, I79C, K125V, F158G, S175V, S177T, T180R,
R190A, S194A, N235C, F238W, C277M, E284K, A293V
P5G, D10L, C41G, F49L, W61V, F64W, I79C, K125M, F158G, S175A, S177T, T180V,
R190A, S194A, N235C, F238L, N11D, C277M, E284K, A293G
P5G, D10V, C41A, F49R, W61A, F64M, I79C, K125W, F158G, S175A, S177A, T180R
P5V, D10V, C41S, F49M, W61A, F64M, I79A, K125W, F158G, S175A, S177G, T180R,
R190S, S194V, N235K, F238L, C277M, E284R, A293G
P5V, D10V, C41A, F49R, W61A, F64L, I79A, K125M, F158G, S175T, S177G, T180V,
R190Q, S194A, N235C, F238L, C277M, E284K, A293V
P5G, D10V, C41A, F49L, W61V, F64G, I79A, K125V, A129T, F158G, S175V, S177A,
T180R, R190Q, S194L, N235V, F238L, C277M, E284D, A293G
P5G, D10V, C41A, F49L, W61V, F64L, I79A, K125V, F158G, S175A, S177T, T180L,
R190A, S194A, N11D, N235C, F238L, C277M, E284D, A293G
P5V, D10V, C41A, F49L, W61V, F64T, I79C, K125M, F158G, S175A, S177A, T180L,
R190Q, S194A, N235V, F238W, C277M, E284K, A293G
P5V, D10W, C41S, F49M, W61A, F64L, I79A, K125W, F158G, S175A, S177T, T180V,
R190S, S194A, N235K, F238L, C277A, E284D, A293K
P5V, D10L, C41A, F49L, W61A, F64M, I79C, K125M, F158G, S175A, S177A, T180L,
R190S, S194L, N235C, F238L, C277A, E284R, A293G
P5G, D10V, C41S, F49L, W61V, F64L, I79A, K125W, F158G, S175A, S177G, T180R,
R190S, S194A, N235C, F238L, C277M, E284R, A293G
P5V, D10V, C41A, F49L, W61V, F64M, I79A, K125W, F158G, S175A, S177G, T180V,
R190G, S194A, N235C, F238W, C277A, E284R, A293V
P5V, D10V, C41S, F49M, W61V, F64G, I79C, K125W, F158A, S175T, S177G, T180V,
R190S, S194A, N235K, F238W, C277A, E284D, A293G
P5V, D10W, C41S, F49L, W61A, F64L, I79C, K125M, F138I, F158G, S175A, S177A,
T180L, R190S, S194V, N235K, F238W, C277M, E284R, A293G
C41S, F49R, W61A, F64L, I79C, K125V, F158A, S175V, S177A, T180R, R190S, S194L,
N235C, F238W, C277A, E284D, A293G
P5G, D10V, C41A, F49M, W61A, F64G, I79A, K125W, F158G, S175G, S177T, T180R,
R190S, S194V, N235K, F238L, C277A, E284R, A293G
P5G, D10V, C41S, F49L, W61A, F64G, I79A, K125W, F158G, S175A, S177G, T180L,
R190Q, S194V, N235C, F238W, C277M, E284K, A293G
P5G, D10V, C41S, F49M, W61A, F64M, I79A, K125W, F158A, S175A, S177T, T180V,
R190S, S194L, N235C, F238L, C277M, E284D, A293K
P5G, D10V, C41A, F49M, W61A, F64L, I79C, K125M, A129T, F158G, S175G, S177A,
T180R, R190A, S194L, N235K, F238L, C277A, E284D, A293G
P5G, D10L, C41S, F49M, W61V, F64W, I79C, K125V, F158A, S175T, S177T, T180R,
R190S, S194A, N235K, F238L, C277A, E284R, A293G
P5V, D10W, C41G, F49R, W61V, F64T, I79A, K125V, F158G, S175T, S177A, T180V,
R190S, S194V, N235K, F238L, C277M, E284D, A293G
P5G, D10V, C41A, F49L, W61A, F64M, I79C, K125M, F158G, S175V, S177A, T180V,
R190Q, S194A, N235V, F238W, C277M, E284D, A293G
P5V, D10W, C41S, F49M, W61A, F64T, I79C, K125M, F158G, S175V, S177T, T180V,
R190A, S194A, N235K, F238W, C277A, E284D, A293K
C41A, F49L, W61A, F64T, I79A, K125M, F158A, S175V, S177G, T180R, R190Q,
S194A, N235C, F238W, C277M, E284D, A293G
P5G, D10L, C41S, F49M, W61V, F64M, I79C, K125W, F158A, S175A, S177G, T180R,
R190S, S194A, N235K, F238W, C277M, E284D, A293G
P5V, D10W, C41G, F49R, W61A, F64M, I79A, K125V, F158A, S175V, S177T, T180R,
R190S, S194V, N235C, F238L, C277M, E284R, A293K
P5V, D10V, C41A, F49L, W61V, F64M, I79A, K125W, F158A, S175A, S177G, T180R,
R190S, S194V, N235C, F238L, C277A, E284D, A293G
P5G, D10W, C41G, F49L, W61V, F64W, I79C, K125W, F158G, S175A, S177T, T180V,
R190A, S194A, N235V, F238W, C277M, E284K, A293K
P5G, D10V, C41S, F49M, W61V, F64W, I79A, K125W, F158G, S175V, S177T, T180R,
R190Q, S194A, N235C, F238L, C277A, E284D, A293K
P5G, D10L, C41G, F49L, W61A, F64M, I79A, K125V, F158A, S175A, S177G, T180V,
R190G, S194L, N235V, F238W, C277M, E284R, A286G, A293G
P5G, D10W, C41A, F49L, W61A, F64M, I79A, K125W, F158G, S175G, S177T, T180L,
R190Q, S194A, N235K, F238L, C277A, E284R, A293G
P5V, D10L, C41S, F49L, W61A, F64L, I79C, K125M, F158A, S175G, S177A, T180R,
R190Q, S194V, N235C, F238L, C277M, E284R, A293G
C41S, F49L, W61A, F64M, I79A, K125M, F158A, S175V, S177T, T180R, R190G,
S194L, N235V, F238L, C277A, E284D, A293G
P5G, D10V, C41A, F49M, W61A, F64T, I79C, K125V, F158A, S175V, S177A, T180L,
R190S, S194A, N235V, F238W, C277M, E284R, A293G
P5G, D10V, C41G, F49R, W61A, F64T, I79A, K125W, F158A, S175G, S177T, T180L,
R190S, S194L, N235C, F238L, C277M, E284D, A293G
P5G, D10W, C41A, F49M, W61V, F64T, I79C, K125M, F158A, S175T, S177A, T180L,
R190A, S194L, N235C, F238W, C277M, E284D, A293G
P5G, D10L, C41A, F49L, W61A, F64L, I79A, K125W, F158A, S175G, S177T, T180V,
R190A, S194A, N235C, F238W, C277M, E284R, A293G
P5G, D10L, C41A, F49M, W61A, F64L, I79C, K125V, F158A, S175G, S177A, T180V,
R190A, F195V, S194L, N235C, F238W, C277A, E284D, A293G
P5V, D10L, C41G, F49M, W61V, F64T, I79C, K125W, F158A, S175T, S177A, T180V,
R190S, S194V, N235C, F238W, C277A, E284D, A293G
P5G, D10W, C41S, F49L, W61V, F64L, I79A, K125W, F158A, S175A, S177G, T180R,
R190G, S194L, N235C, F238W, C277A, E284R, A293G
P5G, D10V, C41A, F49M, W61A, F64G, I79A, K125V, F158G, S175A, S177A, T180L,
R190Q, S194L, N235C, F238W, C277M, E284D, A293V
P5G, D10W, C41G, F49M, W61A, F64W, I79C, K125M, F158G, S175A, S177A, T180L,
R190A, S194L, N235K, F238W, C277M, E284D, A293G
P5V, D10W, C41G, F49L, W61A, F64W, I79C, K125V, F158G, S175G, S177G, T180L,
R190S, S194A, N235K, F238L, C277A, E284D, A293V
P5G, D10W, C41A, F49L, W61A, F64G, I79C, K125W, F158A, S175A, S177A, T180V,
R190S, S194A, N235K, F238W, C277A, E284R, A293V
P5V, D10L, C41S, F49L, W61A, F64M, I79A, K125W, F158A, S175A, S177A, T180L,
R190S, S194L, N235C, F238L, C277A, E284D, A293G
P5G, D10L, C41A, F49L, W61A, F64M, I79C, K125V, F158A, S175V, S177T, T180R,
R190A, S194A, N235C, F238W, C277M, E284K, A293G
P5G, D10V, C41S, F49L, W61A, F64T, I79C, K125M, F158A, S175T, S177T, T180L,
R190Q, S194L, N235C, F238L, C277A, E284D, A293G
K125W, F158A, S175A, S177T, T180L, R190Q, S194A, N235C, F238L, C277A, E284D,
A293V
P5G, D10V, C41A, F49L, W61V, F64L, I79A, K125M, F158A, S175G, S177G, T180L,
R190G, S194V, N235K, F238L, C277A, E284R, A293K
P5V, D10L, C41G, F49L, W61V, F64T, I79C, K125V, F158G, S175G, S177T, T180R,
R190S, S194L, N235C, F238L, C277M, E284R, A293G
P5V, D10V, C41S, F49M, W61A, F64M, I79A, K125W, F158A, S175A, S177G, T180L,
R190G, S194A, N235C, F238L, C277A, E284D, A293V
P5G, D10W, C41S, F49M, W61A, F64W, I79C, K125W, F158A, S175A, S177A, T180R,
R190S, S194A, N235C, F238W, C277M, E284D, A293G
P5V, D10V, C41G, F49L, W61V, F64M, I79C, K125V, F158A, S175G, S177T, T180L,
R190Q, S194A, N235V, F238L, C277M, E284R, A293G
P5V, D10L, C41S, F49L, W61A, F64T, I79C, K125M, F158G, S175G, S177A, T180L,
R190A, S194A, N235C, F238L, C277M, E284K, A293G
P5G, D10V, C41A, F49L, W61A, F64G, I79C, K125W, F158A, S175A, S177T, T180R,
R190S, S194V, N235C, F238W, C277A, E284D, A293G
P5G, D10W, C41G, F49R, W61A, F64L, I79C, K125M, F158A, S175A, S177A, T180L,
R190A, S194V, N235C, F238W, C277M, E284D, A293V
C41A, F49L, W61V, F64L, I79A, K125V, F158G, S175G, S177G, T180L, R190G,
S194A, N235V, F238W, C277A, E284D, A293G
K125W, F158A, S175A, S177G, T180V, R190S, S194L, N235K, F238W, C277M,
E284R, A293G
P5V, D10V, C41S, F49M, W61A, F64M, I79C, K125M, F158A, S175G, S177T, T180R,
R190S, S194A, N235V, F238W, C277A, E284R, A293G
P5G, D10W, C41A, F49L, W61A, F64L, I79C, K125W, F158A, S175A, S177G, T180V,
R190A, S194A, N235C, F238W, C277A, E284D, A293G
P5G, D10W, C41S, F49L, W61A, F64W, I79A, K125V, F158A, S175T, S177G, T180V,
R190A, S194L, N235V, F238L, C277M, E284D, A293V
P5V, D10W, C41G, F49L, W61A, F64G, I79A, K125M, F158A, S175G, S177A, T180L,
R190S, S194V, N235V, F238L, C277A, E284D, A293K
P5G, D10L, C41A, F49L, W61A, F64W, I79C, K125W, F158A, S175V, S177A, T180L,
R190S, S194L, N235K, F238W, C277M, E284D, A293G
P5V, D10L, C41G, F49R, W61V, F64M, I79A, K125W, F158A, S175T, S177A, T180L,
R190Q, S194L, N235C, F238W, C277M, E284K, A293V
P5V, D10W, C41G, F49L, W61A, F64G, I79A, K125V, F158A, S175T, S177G, T180V,
R190Q, S194V, N235V, F238W, C277M, E284K, A293G
P5V, D10W, C41G, F49R, W61A, F64L, I79A, K125M, F158G, S175G, S177A, T180L,
R190S, S194L, N235C, F238W, C277M, E284D, A293G
P5V, D10W, C41G, F49R, W61A, F64T, I79A, K125V, F158A, S175A, S177T, T180R,
R190G, S194V, N235K, F238W, C277M, E284D, A293G
P5G, D10W, C41S, F49L, W61A, F64T, I79C, K125V, F158A, S175G, S177A, T180L,
R190S, S194A, N235V, F238L, C277A, E284D, A293G
P5V, D10W, C41G, F49L, W61A, F64M, I79A, K125V, F158A, S175G, S177G, T180L,
R190Q, S194L, N235C, F238L, C277A, E284D, A293V
P5G, D10V, C41S, F49M, W61A, F64W, I79C, K125V, F158G, S175G, S177T, T180R,
R190A, S194L, N235C, F238L, C277M, E284R, A293G
P5G, D10V, C41G, F49R, W61V, F64M, I79A, K125V, F158G, S175G, S177G, T180V,
R190S, S194V, N235K, F238W, C277A, E284K, A293G
P5G, D10L, C41S, F49L, W61V, F64L, I79A, K125M, F158A, S175G, S177G, T180V,
R190A, S194A, N235K, F238W, C277M, E284D, A293G
F49R, W61V, F64M, I79A, K125W, F158A, S175G, S177A, T180V, R190Q, S194V,
N235V, F238W, C277M, E284R, A293G

5. The polypeptide of claim 1, wherein the polypeptide is encoded by a polynucleotide sequence having at least 80% identity to SEQ ID NO: 19, and a silent codon difference as compared to SEQ ID NO: 19 at a position encoding an amino acid residue selected from: V33, I37, F73, N74, A78, Q82, K93, P97, V99, S104, L111, L117, G119, F132, V133, I137, G139, F141, R152, Q155, N160, S166, A182, T201, G218, I213, V224, S225, A233, G242, V261, K263, F276, S295, L304, Y306, F311, and V312;
optionally, wherein the codon differences are selected from: V33 (GTT>GTC), I37 (ATT>ATC), F73 (TTT>TTC), N74 (AAT>AAC), A78 (GCA>GCG), Q82 (CAA>CAG), K93 (AAG>AAA), P97 (CCA>CCG), V99 (GTT>GTC), S104 (TCA>TCT), L111 (TTA>TTG), L117 (TTG>CTG), G119 (GGT>GGC), F132F (TTC>TTT), V133 (GTT>GTC), G139 (GGT>GGG), R152 (AGA>CGT), Q155 (CAA>CAG), N160 (AAT>AAC), L162 (TTG>CTG), S166 (TCT>TCC), A182 (GCA>GCC), T201 (ACT>ACG), I213 (ATC>ATT), G218 (GGT>GGG), V224 (GTT>GTC), S225 (TCA>TCG), A233 (GCA>GCG), G242 (GGT>GGC), V261 (GTT>GTC), K263 (AAA>AAG), F276 (TTC>TTT), S295 (TCA>TCT), L304 (TTG>CTG), Y306 (TAT>TAC), F311 (TTT>TTC), and V312 (GTT>GTC).

6. The polypeptide of claim 1, wherein the polypeptide comprises an amino acid sequence of at least 80% identity to a sequence selected from the group consisting of SEQ ID NO: 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, and 514.

7. The polypeptide of claim 1, wherein the polypeptide further comprises an N-terminal truncation of from 2 to 12 amino acids as compared to SEQ ID NO: 20;
optionally, wherein, the polypeptide comprises an amino acid sequence of at least 80% identity to a sequence selected from the group consisting of SEQ ID NO: 516, 518, 520, 522, and 524.

8. The polypeptide of claim 1 in which the prenyltransferase activity of the polypeptide as compared to a polypeptide consisting of SEQ ID NO: 20 is increased at least 1.2-fold.

9. The polypeptide of claim 8 in which the prenyltransferase activity is measured as the rate of conversion of the substrates olivetolic acid (OA) and geranyl pyrophosphate (GPP) to cannabigerolic acid (CBGA); optionally, under reaction conditions of pH 7 and 30 C.

10. A polynucleotide encoding the polypeptide of claim 1; optionally, wherein the polynucleotide sequence
(a) has at least 80% identity to a sequence selected from the group consisting of SEQ ID NO: 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, and 513; or
(b) is a codon degenerate sequence of a sequence selected from the group consisting of SEQ ID NO: 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, and 513.

11. A host cell comprising the polynucleotide of claim 10.

12. A method for preparing a recombinant polypeptide having prenyltransferase activity comprising:
(a) transforming a host cell with an expression vector comprising a polynucleotide encoding a recombinant polypeptide of claim 10;
(b) culturing said transformed host cell under conditions whereby said recombinant polypeptide is produced by said host cell; and
(c) recovering said recombinant polypeptide from said host cells.

13. A recombinant host cell comprising a nucleic acid encoding a recombinant polypeptide having prenyltransferase activity of claim 1.

14. The host cell of claim 13, wherein the nucleic acid encodes a N-terminal fusion of the Erg20ww polypeptide of SEQ ID NO: 526 and the recombinant polypeptide having prenyltransferase activity of claim 1.

15. The host cell of claim 13, wherein the host cell further comprises a pathway of enzymes capable of converting hexanoic acid (HA) to olivetolic acid (OA); optionally, wherein the pathway comprises at least the enzymes AAE, OLS, and OAC.

16. The host cell of claim 13, wherein the cell produces the cannabinoid, CBGA; optionally, wherein the production of CBGA is increased at least 2-fold, at least 3-fold, at least 4-fold, at least 5-fold, or more, relative to a control recombinant host cell comprising a pathway with the recombinant polypeptide having prenyltransferase activity replaced by a polypeptide of SEQ ID NO: 20.

17. The host cell of claim 13, wherein recombinant host cell source is selected from Saccharomyces cerevisiae, Yarrowia lipolytica, Pichia pastoris, and Escherichia coli.

18. The host cell of claim 13, wherein the nucleic acid is integrated in the host cell genome at a locus selected from: NDE1, XII-5, Gal80, ROQ1; optionally, wherein the nucleic acid is integrated in the host cell genome at two loci selected from: XII-5 and NDE1; or ROQ1 and NDE1.

19. A method for producing a cannabinoid comprising:
(a) culturing in a suitable medium a recombinant host cell of claim 13; and
(b) recovering the produced cannabinoid.

20. A method for preparing a compound of structural formula (I) [symbol]
(I)
wherein, R¹ is C1-C7 alkyl,
comprising contacting under suitable reactions conditions geranyl pyrophosphate (GPP) and a compound of structural formula (II) 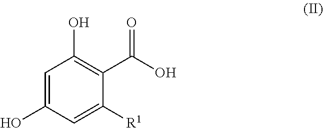
wherein, R¹ is C1-C7 alkyl,
and a recombinant polypeptide of claim 1.
